(19) United States
(12) Patent Application Publication
(10) Pub. No.: US 2024/0385168 A1
(43) Pub. Date: Nov. 21, 2024

(54) SEMICONDUCTOR-BASED MEASUREMENT DEVICES

(71) Applicant: CytoTronics, Boston, MA (US)

(72) Inventors: Alexander Alexeyev, Boston, MA (US); Mickey Cheah, Boston, MA (US); Zach Boynton, Boston, MA (US); Wenxuan Wu, Boston, MA (US); Jeffrey Abbott, Boston, MA (US)

(21) Appl. No.: 18/668,097

(22) Filed: May 17, 2024

Related U.S. Application Data
(60) Provisional application No. 63/467,595, filed on May 18, 2023.

(51) Int. Cl. G01N 33/483; G06F 13/42

(52) U.S. Cl. CPC G01N 33/4836; G06F 13/4282

(57) ABSTRACT

Measurement devices include: a substrate; a plurality of integrated circuits, each featuring a plurality of electrodes, and the plurality of integrated circuits being positioned on the substrate in a two-dimensional array including n rows and m columns; a communication interface connected to each of the plurality of integrated circuits so that each integrated circuit is addressable through the interface; and a data output interface featuring multiple data output lines connected to the communication interface, where in each of the n rows of the array, the integrated circuits are connected to a common data line, and the measurement device is configurable, responsive to a control signal, to transmit measurement information through the data output interface by, in one or more of the n rows of the array, selectively transmitting measurement information from one of the integrated circuits connected to the row's common data line to the data output interface.

### CROSS-REFERENCE TO RELATED APPLICATIONS

This application claims priority to U.S. Provisional Application No. 63/467,595 filed on May 18, 2023, the entire contents of which are incorporated herein by reference.

### TECHNICAL FIELD

This disclosure relates to semiconductor-based devices for measuring electrical parameters and responses in biological samples, systems that include such devices, and methods associated with the devices and systems.

### BACKGROUND

Optical methods have been used to image cell proliferation and death for characterizing the efficacy of a variety of different therapeutic substances and methods. More recently, electrical and electrochemical measurements have been used to elucidate cellular responses to stimuli that are not conveniently measured by optical methods, such as cell adhesion.

Intracellular and extracellular measurements of membrane potentials and other parameters of networks of electrogenic cells have been made using CMOS-based microelectrode arrays and planar patch-clamp arrays. Sensitivity limitations have restricted the ability of such arrays to detect certain cellular responses, and bandwidth limitations have limited the resolution of electrical and electrochemical measurements performed with such arrays.

### SUMMARY

The present disclosure features measurement devices and systems for measuring electrically induced responses from individual cells and cellular networks. The measurement devices include a plurality of integrated circuits, each of which includes measurement electrodes, reference electrode amplifiers, peripheral circuits, and other circuitry configurable to perform a variety of measurement methods. Voltage signals and current signals can be selectively applied to individual electrodes of the integrated circuits to stimulate a variety of different cellular responses. Groups of electrodes can be selectively scanned to measure responses induced by the applied voltage and current signals. The electrodes are integrated into a substrate, and the cells and cellular networks are typically positioned on or adjacent to the substrate. By controlling the nature and spatial distribution of signals applied to specific electrodes, the electric field geometry at and above the substrate surface can be controlled. Furthermore, by controlling the locations at which voltage and current signals are applied by the electrodes and cellular responses are measured by the electrodes, a variety of different physiological cell parameters can be interrogated.

In one aspect, the disclosure features measurement devices that include: a substrate; a plurality of integrated circuits, each featuring a plurality of electrodes, and the plurality of integrated circuits being positioned on the substrate in a two-dimensional array that includes n rows and m columns; a communication interface connected to each of the plurality of integrated circuits so that each integrated circuit is addressable through the interface; and a data output interface featuring multiple data output lines connected to the communication interface, where in each of the n rows of the array, the integrated circuits are connected to a common data line, and the measurement device is configurable, responsive to a control signal, to transmit measurement information through the data output interface by, in one or more of the n rows of the array, selectively transmitting measurement information from one of the integrated circuits connected to the row's common data line to the data output interface, and inactivating one or more of the other integrated circuits connected to the row's common data line.

In another aspect, the disclosure features methods that include: providing a measurement device that includes a substrate, a plurality of integrated circuits, each featuring a plurality of electrodes, and the plurality of integrated circuits being positioned on the substrate in a two-dimensional array that includes n rows and m columns, a communication interface connected to each of the plurality of integrated circuits so that each integrated circuit is addressable through the interface, and a data output interface featuring multiple data output lines connected to the communication interface, where in each of the n rows of the array, the integrated circuits are connected to a common data line; selectively transmitting measurement information from one of the integrated circuits connected to the row's common data line to the data output interface; and inactivating one or more of the other integrated circuits connected to the row's common data line.

In a further aspect, the disclosure features systems that include: a measurement device featuring a substrate, a plurality of integrated circuits on the substrate, where each integrated circuit includes a plurality of electrodes and at least one analog-to-digital converter (ADC), and a data output interface connected to the at least one ADC and featuring multiple data output lines, where the at least one ADC is configured to receive measurement signals from one or more of the plurality of electrodes and to generate one or more data signals that include measurement information derived from the measurement signals; and a host controller that includes a host interface configured to connect to the data output interface of the measurement device, and instructions that, when executed by the host controller, cause the host controller to obtain measurement information from the measurement device by determining a rate at which measurement information in the one or more data signals will be transmitted from the data output interface to the host interface, selectively activating a number of the data output lines of the data output interface so that a data transfer capacity of the activated data output lines is at least as large as the rate at which measurement information in the one or more data signals will be transmitted from the data output interface to the host interface, and receiving the measurement information in the one or more data signals on the activated data output lines of the data output interface.

In another aspect, the disclosure features methods that include: providing a measurement device that includes a substrate, a plurality of integrated circuits on the substrate, where each integrated circuit features a plurality of electrodes and at least one analog-to-digital converter (ADC), and a data output interface connected to the at least one ADC and featuring multiple data output lines, where the at least one ADC is configured to receive measurement signals from one or more of the plurality of electrodes and to generate one or more data signals that include measurement information derived from the measurement signals; determining a rate at which measurement information in the one or more data signals will be transmitted from the data output interface; selectively activating a number of the data output lines of the data output interface so that a data transfer capacity of the activated data output lines is at least as large as the rate at which measurement information in the one or more data signals will be transmitted from the data output interface; and receiving the measurement information in the one or more data signals on the activated data output lines of the data output interface.

In a further aspect, the disclosure features measurement devices that include a substrate and a plurality of integrated circuits on the substrate, where each integrated circuit includes: a plurality of electrodes; a plurality of peripheral circuits (PCs), where each of the peripheral circuits is connected to multiple electrodes of the integrated circuit; a plurality of analog-to-digital converters (ADCs), where each of the ADCs is connected to multiple PCs through a multiplexer; a data aggregation unit connected to each of the ADCs; and a data output interface that includes multiple data output lines, where the multiple data output lines are connected to the data aggregation unit, and where in each integrated circuit, a total number of PCs exceeds a total number of ADCs, the data output interface is configured to transmit a data clock signal, and the data output interface is configured to transmit measurement information generated by the ADCs from measurement signals detected at electrodes of the integrated circuit in a plurality of sequential cycles synchronized to the data clock signal.

In another aspect, the disclosure features methods that include: providing a measurement device that includes a substrate and a plurality of integrated circuits on the substrate, where each integrated circuit features a plurality of electrodes, a plurality of peripheral circuits (PCs), where each of the peripheral circuits is connected to multiple electrodes of the integrated circuit, a plurality of analog-to-digital converters (ADCs), where each of the ADCs is connected to multiple PCs through a multiplexer, a data aggregation unit connected to each of the ADCs, and a data output interface featuring multiple data output lines, where the multiple data output lines are connected to the data aggregation unit, and where in each integrated circuit a total number of PCs exceeds a total number of ADCs; transmitting a data clock signal via the data output interface; and transmitting measurement information generated by the ADCs from measurement signals detected at electrodes of the integrated circuit via the data output interface in a plurality of sequential cycles synchronized to the data clock signal.

In a further aspect, the disclosure features measurement devices that include a substrate and a plurality of integrated circuits on the substrate, each integrated circuit featuring a plurality of electrodes and connected to a pixel circuit that is independently switchable among multiple operating modes, where the measurement device is configurable, responsive to a control signal, to: deliver a stimulus signal external to the measurement device using one or more electrodes by adjusting pixel circuits connected to the one or more electrodes to operate in a first mode; and detect a measurement signal at the one or more electrodes by adjusting the pixel circuits to operate in a second mode different from the first mode.

In another aspect, the disclosure features methods that include: providing a measurement device featuring a substrate and a plurality of integrated circuits on the substrate, each integrated circuit including a plurality of electrodes and connected to a pixel circuit that is independently switchable among multiple operating modes; delivering a stimulus signal external to the measurement device using one or more electrodes by adjusting pixel circuits connected to the one or more electrodes to operate in a first mode; and detecting a measurement signal at the one or more electrodes by adjusting the pixel circuits to operate in a second mode different from the first mode.

In a further aspect, the disclosure features measurement devices that include a substrate and a plurality of integrated circuits on the substrate, each integrated circuit including a plurality of electrodes positioned in a two-dimensional array on the substrate, and each electrode being connected to an independent pixel circuit, where for each of one or more of the plurality of integrated circuits, the measurement device is configurable, responsive to a control signal, to: receive information from the control signal designating an effective electrode pitch for measurement electrodes; and configure the pixel circuits connected to a set of electrodes of the integrated circuit to cause the electrodes of the set to operate as a regularly-spaced array of measurement electrodes at which measurement signals are detectable, and where an electrode pitch of the array of measurement electrodes matches the effective electrode pitch.

In another aspect, the disclosure features methods that include providing a measurement device including a substrate and a plurality of integrated circuits on the substrate, each integrated circuit featuring a plurality of electrodes positioned in a two-dimensional array on the substrate, and each electrode being connected to an independent pixel circuit, and for each of one or more of the plurality of integrated circuits: receiving information from the control signal designating an effective electrode pitch for measurement electrodes; and configuring the pixel circuits connected to a set of electrodes of the integrated circuit to cause the electrodes of the set to operate as a regularly-spaced array of measurement electrodes at which measurement signals are detectable, where an electrode pitch of the array of measurement electrodes matches the effective electrode pitch.

In a further aspect, the disclosure features measurement devices that include a substrate and a plurality of integrated circuits on the substrate, where each integrated circuit includes a plurality of electrodes, a plurality of peripheral circuits, a plurality of reference electrode amplifiers, and at least one digital-to-analog converter (DAC) configurable, responsive to a control signal, to selectively connect to one or more of the plurality of peripheral circuits and to one or more of the reference electrode amplifiers, and the peripheral circuits, the reference electrode amplifiers, and the at least one DAC are configurable, responsive to a control signal, to control an electric field distribution adjacent to the substrate by: delivering one or more driving signals to a first subset of the plurality of electrodes; delivering one or more reference signals to a second subset of the plurality of electrodes different from the first subset; and delivering one or more shielding signals to a third subset of the plurality of electrodes that is different from the first and second subsets.

In another aspect, the disclosure features methods that include: providing a measurement device featuring a substrate and a plurality of integrated circuits on the substrate, where each integrated circuit includes a plurality of electrodes, a plurality of peripheral circuits, a plurality of reference electrode amplifiers, and at least one digital-to-analog converter (DAC) configurable, responsive to a control signal, to selectively connect to one or more of the plurality of peripheral circuits and to one or more of the reference electrode amplifiers; and controlling an electric field distribution adjacent to the substrate by delivering one or more driving signals to a first subset of the plurality of electrodes, delivering one or more reference signals to a second subset of the plurality of electrodes different from the first subset, and delivering one or more shielding signals to a third subset of the plurality of electrodes that is different from the first and second subsets.

In a further aspect, the disclosure features measurement devices that include a substrate and a plurality of integrated circuits on the substrate, where each integrated circuit includes a plurality of electrodes, at least one digital-to-analog converter (DAC), a plurality of peripheral circuits, and a plurality of reference electrode amplifiers, and where: each peripheral circuit and reference electrode amplifier is configurable to selectively connect to the at least one DAC; and the at least one DAC is configurable, responsive to a control signal, to generate a driving signal for one or more of the plurality of electrodes comprising multiple frequency components.

In another aspect, the disclosure features methods that include: providing a measurement device that includes a substrate and a plurality of integrated circuits on the substrate, where each integrated circuit features a plurality of electrodes, at least one digital-to-analog converter (DAC), a plurality of peripheral circuits, and a plurality of reference electrode amplifiers, and where each peripheral circuit and reference electrode amplifier is configurable to selectively connect to the at least one DAC; and generating, using the at least one DAC, a driving signal for one or more of the plurality of electrodes comprising multiple frequency components.

Embodiments of any of the measurement devices, systems, and methods can include any of the features described herein, including any combination of features that are individually described in connection with different embodiments, unless specifically stated otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description, drawings, and claims.

### DESCRIPTION OF DRAWINGS

FIG. 1 is a schematic diagram of an example of a measurement device.

FIG. 2 is a schematic cross-sectional diagram of an example portion of a measurement device.

FIG. 3 is a schematic view of an example of an enclosure.

FIG. 4 is a schematic view of another example of an enclosure.

FIG. 5A is a schematic diagram of an example of a well on a substrate of a measurement device.

FIG. 5B is a schematic diagram of an example of four wells on a substrate of a measurement device.

FIG. 5C is a schematic diagram of an example of 16 wells on a substrate of a measurement device.

FIG. 6 is a schematic diagram of an example of multiple wells on a substrate of a measurement device.

FIG. 7A is a schematic view of an example of a housing and interface for a measurement device.

FIG. 7B is a schematic view of an example of a housing array for measurement devices.

FIGS. 8A-8D are schematic cross-sectional views of different methods for attaching integrated circuit chips to a substrate of a measurement device.

FIG. 9 is a schematic diagram of an example integrated circuit of a measurement device.

FIG. 10A is a schematic diagram of an example pixel circuit of a measurement device.

FIG. 10B is a schematic diagram of an example D-latch of a pixel circuit.

FIG. 10C is a schematic diagram of an example pixel circuit of a measurement device.

FIG. 11A is a schematic diagram of an example configuration of the pixel circuit of FIG. 10c.

FIG. 11B is a plot showing examples of two clock signals for a current injector of the pixel circuit of FIG. 10C.

FIGS. 11C-11I are schematic diagrams of other example configurations of the pixel circuit of FIG. 10C.

FIGS. 12A-12D are schematic diagrams of a portion of an electrode array configured to define measurement arrays of electrodes with different electrode pitches.

FIG. 13 is a schematic diagram of an example of an integrated circuit with multiple functional groups.

FIG. 14 is a schematic diagram of an example of an analog-to-digital converter (ADC) connected to a multiplexer.

FIG. 15 is a schematic diagram of an example of a peripheral circuit of a measurement device.

FIGS. 16A-16F are schematic diagrams of example configurations of the peripheral circuit of FIG. 15.

FIG. 17 is a schematic diagram of an example of a digital-to-analog converter (DAC) of a measurement device.

FIG. 18A is a schematic diagram of an example of a resistive string module of the DAC of FIG. 17.

FIG. 18B is a schematic diagram of an example of a current source of the DAC of FIG. 17.

FIG. 18C is a schematic diagram of an example of a transimpedance amplifier (TIA) of the DAC of FIG. 17.

FIG. 19 is a schematic diagram of an example measurement device.

FIG. 20 is another schematic diagram of an example measurement device.

FIG. 21 is a flow chart showing a series of example steps for measuring responses of one or more samples to electrical stimuli.

FIG. 22 is a schematic diagram showing an example measurement device with multiple samples located on the device.

FIG. 23 is a schematic cross-sectional view of an example of a measurement configuration in which the response of a sample to a vertically-oriented electric field is interrogated.

FIG. 24 is a schematic diagram showing two measurement frames for measuring sample responses to electrical stimuli in the configuration of FIG. 23.

FIG. 25 a schematic cross-sectional view of an example of a measurement configuration in which the response of a sample to a laterally-oriented electric field is interrogated.

FIG. 26 is a schematic diagram showing two measurement frames for measuring sample responses to electrical stimuli in the configuration of FIG. 25.

FIG. 27A is a set of schematic diagrams showing vertical field and lateral field configurations for measuring sample responses, and the results of calculations of confluency and root-mean-square (RMS) frame-to-frame movement of the samples. Examples of measured electric field lines are shown between stimulation (dark gray) and return electrodes (light gray) and potential distribution in blue. At low-frequency (250 Hz), cell membranes block the electric fields whereas at higher frequency (16 kHz) the fields become more proximately sensitive. Confluency and RMS are calculated from the impedance images.

FIG. 27B is a set of graphs showing measured and calculated parameter values for MDCK cells obtained from canine kidney. Cells were seeded at time t=0 hours at a density of 40,000 cells/well and measured for 40 hours. Media was changed at 24 hours post-seeding. The traces represent mean±s.d. for 88 wells across the measurement device. The most representative biological parameter is labeled for each measurement, but a blend of biological parameters contributes to each measurement in total.

FIG. 27C is a set of impedance images of MDCK cells from a single well at multiple time points. The images were generated with VF 250 Hz, VF 16 kHz, and the inverse of the LF 16 kHz.

FIG. 28A is a set of graphs showing live-cell impedance measurements for eleven cell types. MDCK cell variants are canine, the remainder are human. Violin plots of three parameters show differences in tissue barrier (VF 250 Hz), cell-substrate attachment (LF 16 kHz), and movement/dynamic changes in morphology (RMS).

FIG. 28B is a set of immunofluorescence images (left side) and impedance images (right side) for various cell types, showing a range of epithelial tissue properties. Cells were stained for E-Cadherin, a cell-cell adhesion protein, and for DAPI/nucleus. A representative well impedance image combines several impedance parameters into different shades of grey. Cells strongly expressing E-Cadherin at cell-cell interfaces (MDCK, Caco-2, MCF-7) show high tissue barrier in comparison to cells with dispersed E-Cadherin (A549) or no expression (MDA-MB-231).

FIG. 28C is a set of graphs showing measured and calculated parameter values for Caco-2 cells plated on uncoated and collagen-coated substrates of a measurement device. Differences in Caco-2 cell function from 0-48 hours after seeding were observed. Tissues on coated surfaces increased in tissue barrier (VF 250 Hz) more significantly with a media exchange and showed less attachment (LF 16 kHz). Line traces represent mean±s.d. for 9 wells per condition; bar graphs are at 48 hours (p<0.0001, n=9 wells).

FIG. 28D is a set of graphs showing measured and calculated parameter values for mixtures of two different cell types. Co-culture sensitivity was characterized by mixing two breast cancer cell types of differing functional properties, MCF-7 and MDA-MB-231, plated at a total of 10,000 cells/well. Gradients correlating to plating ratios are observed for cell size (VF 16 kHz) and RMS (movement). Line traces represent mean±s.d. for 12 wells per condition.

FIGS. 28E-28O are a set of graphs showing measured and calculated parameter values for different cell types. The total number of cells plated per well for each of cell type is indicated. Media changes were performed either around 24 or 48 hours depending upon cell type and experiment. The y-axis of each graph is scaled on a per cell type basis.

FIG. 29A is a set of images showing measured and calculated parameter values for MDCK cells treated with two different compounds, Alisertib and Bosutinib. MDCK cells were obtained from canine kidney. Alisertib was applied at a concentration of 1 μM and Bosutinib was applied at a concentration of 10 μM. Domes were only observed for tissues treated with Alisertib.

FIG. 29B is a schematic diagram showing a greyscale map for the images of FIG. 29A.

FIG. 29C is a graph showing measurements of cell attachment as a function of time for sample to which Alisertib or Bosutinib was applied. Alisertib accelerated and Bosutinib delayed the water transport detachment (LF 16 kHz) with respect to the control; mean±s.d. for 3 wells per condition. The differential results match animal study results for autosomal dominant polycystic kidney disease (ADPKD), suggesting the water transport phenotype could be predictive of ADPKD efficacy.

FIGS. 29D-1 through 29D-10 are a set of graphs showing measured and calculated parameter values for MDCK cells to which a range of compounds were applied prior to water transport turning on at approximately 24 hours post-plating. Concentrations of compounds are indicated in the legends and values are time-normalized to 1 hour before compound application.

FIGS. 29E-1 through 29E-10 are a set of graphs showing measured and calculated parameter values for A549 cells to which a range of compounds were applied prior to water transport turning on at approximately 24 hours post-plating. Concentrations of compounds are indicated in the legends and values are time-normalized to 1 hour before compound application.

FIGS. 29F-1 through 29F-10 are a set of graphs showing measured and calculated parameter values for MDA-MB-231 cells to which a range of compounds were applied prior to water transport turning on at approximately 24 hours post-plating. Concentrations of compounds are indicated in the legends and values are time-normalized to 1 hour before compound application.

FIG. 30A is a graph showing results of a principal components analysis of multiparametric impedance measurements for A549 cells. A549 (lung/alveoli) cells were treated with a library of 341 FDA approved compounds at a concentration of 10 μM; Anisomycin (100 nM) was used as a positive control. A principal component analysis (PCA) was performed using the various field measurements (VF, LF at 1 kHz, 4 kHz, and 16 kHz) and calculations (confluency, RMS) across time points spanning from 0-48 hours post compound treatment. The first two dimensions of PCA space are displayed with select clusters highlighted.

FIG. 30B is a set of graphs showing 48-hour time traces post-compound treatment for a subset of the parameters in the PCA. Each of the principal components shows a unique signature. Shaded lines represent the control (median) response.

FIG. 30C is a table showing compounds for each displayed cluster in FIG. 30A with a target and/or pathway identified from the compound supplier.

FIG. 30D is a set of graphs showing additional clusters identified from the PCA of the multiparametric impedance measurements of FIG. 30A. A variety of compounds decrease growth or induce cell death, and other morphological features distinguish diverse MOAs. The first four dimensions of PCA space are shown in the graphs.

FIG. 30E is a set of graphs showing 48-hour time traces post-compound treatment for a subset of the parameters in the PCA.

FIG. 30F is a table showing compounds for each displayed cluster in FIG. 30D with a target and/or pathway identified from the compound supplier.

FIG. 30G is a set of graphs showing additional clusters identified from the PCA of the multiparametric impedance measurements of FIG. 30A. Proteasome inhibition death phenotypes, anti-proliferation, and anti-viral all reduce cell growth or induce cell death. The first four dimensions of PCA space are shown in the graphs.

FIG. 30H is a set of graphs showing 48-hour time traces post-compound treatment for a subset of the parameters in the PCA.

FIG. 30I is a table showing compounds for each displayed cluster in FIG. 30G with a target and/or pathway identified from the compound supplier.

FIG. 30J is a set of graphs showing additional clusters identified from the PCA of the multiparametric impedance measurements of FIG. 30A. Two Akt/mTOR clusters were observed, both distinguished due to an increase in the RMS (dynamic morphology) parameter. The first four dimensions of PCA space are shown in the graphs.

FIG. 30K is a set of graphs showing 48-hour time traces post-compound treatment for a subset of the parameters in the PCA.

FIG. 30L is a table showing compounds for each displayed cluster in FIG. 30J with a target and/or pathway identified from the compound supplier.

FIG. 31A is a set of graphs showing results of a principal components analysis of multiparametric impedance measurements for Caco-2 cells. A library of 341 FDA approved compounds were applied to Caco-2 cells at a concentration of 10 μM. A principal component analysis (PCA) was performed using the various field measurements (VF, LF at 250 Hz, 1 kHz, 4 kHz, and 16 kHz) and calculations (confluency, RMS) across time points spanning from 0-48 hours post-compound treatment. The first four dimensions of PCA space are shown in the graphs.

FIG. 31B is a set of graphs showing 48-hour time traces post-compound treatment for a subset of the parameters in the PCA. Unique signatures are observed for each of the clusters. Shaded lines represent the control (median) response.

FIG. 31C is a table showing compounds for each displayed cluster in FIG. 31A with a target and/or pathway identified from the compound supplier.

FIG. 31D is a graph showing a maximum value of the VF 250 Hz measurement between 0-48 hours post-compound treatment for Caco-2 cells treated with the 341 FDA approved compounds of FIG. 31A. The compounds were tested against the barrier function phenotype exhibited in Caco-2 cells.

FIG. 31E is a set of graphs showing a dose response of Caco-2 tissues to the compounds which showed a highest observed increase in barrier, Amonafide and Ciclopirox. Concentrations were tested from 1 nM to 10 μM (traces are time normalized to control). The fast versus slow barrier increase for the two compounds suggested different mechanisms of action. Line traces represent mean±s.d. for 3 wells per concentration.

FIG. 31F shows chemical structures of compounds with similar structures to Amonafide.

FIG. 31G is a graph showing changes in the Caco-2 barrier function at 12 hours post-compound application for the different compounds in FIG. 31F. Multiple rounds of screenings show a rapid increase in barrier for Amonafide and NSC 308848; all other compounds showed no significant effect versus control. Bar graphs are at 12 hours (p<1×10⁻¹, n=3 wells). Etoposide represents a similar mechanism of action as Amonafide's specified Topoisomerase II inhibition but is not chemically similar.

FIG. 31H is a set of immunofluorescence images of Caco-2 cells treated with different compounds. Immunofluorescence imaging was performed at 48 hours post-compound treatment for Ciclopirox, Amonafide, and the most similar Amonafide derivatives, NSC 308848 and UNBS5162. Cells were stained for ZO-1, a cell-cell adhesion and signaling protein, for DAPI/nucleus, and Occludin, a cell-cell adhesion protein. Significant increases in ZO-1 in the membrane and nucleus were observed for both Amonafide and NSC 308848. The localization of ZO-1 to the nucleus at 48 hours combined with the rapid increase in impedance barrier suggested an off-target MOA for Amonafide which directly tightens cell-cell junctions resulting in a ZO-1 signaling response.

FIG. 31I is a set of graphs showing impedance measurements for Caco-2 cells treated with the compounds of FIG. 31F. The traces shown are for compounds applied at a concentration of 10 μM concentration. Line traces represent mean for 3 wells per compound. Each compound replicate is plotted independently. Values were time-normalized to 1 hour before compound addition and control-normalized. The dashed line denotes the 12-hour timepoint that corresponds to FIG. 31G.

FIG. 31J is a set of graphs showing measured ZO-1 response in the immunofluorescence images of FIG. 31H at 48-hours post-compound treatment for Cicloprox, Amonafide, and the most similar Amonafide derivatives, NSC 308848 and UNBS5162. ZO-1 (green), a cell-cell adhesion and signaling protein, was quantified using an ImageJ analysis of the membrane intensity (left graph) and nucleus (right graph). The bar graphs represent mean±s.e. for 15 cells per compound or DMSO control (p<0.0001 versus control).

FIG. 32A is a set of impedance images showing Caco-2 cells treated with Bosutinib at different concentrations. A titration of Bosutinib was performed on Caco-2 cells plated on an uncoated surface. A rapid decrease in attachment was observed and domes appeared starting around 40 hours post-compound addition for 10 μM. For the Caco-2 screen of FIGS. 31A-31J, domes were not observed for the Bosutinib positive controls, attributed to the collagen surface treatment increasing cell-substrate attachment to prevent doming.

FIG. 32B is a graph showing measured impedance values for the Caco-2 cells of FIG. 32A at various Bosutinib concentrations as a function of time post-compound treatment. The line traces represent mean±s.d. for 3 wells per condition.

Like reference symbols in the various drawings indicate like elements.

### DETAILED DESCRIPTION

High-throughput, high-sensitivity electrical and electrochemical measurements can be used in a wide variety of applications. For example, by measuring integrated responses of cellular networks to biochemical stimuli, the ability of administered therapeutic agents to affect many different cellular properties—from adhesion to intracellular communication and proliferation or apoptosis—can be determined. Consequently, such measurements find considerable utility in drug screening assays and trials.

As another example, such measurements can be used for chemical sensing and screening applications. Certain detection targets can have reproducible and characteristic electrophysiologic signatures, which are detectable using the methods described herein. Consequently, high-sensitivity measurements are useful for diagnostic applications in which particular target species are identified even when present in low concentrations and/or in complex analytical environments.

This disclosure features measurement devices and systems which can be used for measurement of individual cellular responses and cellular network responses to a variety of complex electrical stimuli. The devices generally include arrays of electrodes that can both induce cellular responses and measure electrical responses of cells. By controlling attributes of the applied electrical stimuli (including, but not limited to, electric field geometry, field intensity and phase, field frequency) and selection of appropriate measurement attributes (such as voltage and current detection modes, near- and far-field detection geometry, and electrode signal readout geometries), many key physiological parameters of cells and cell networks can be elucidated. Certain parameters are challenging to interrogate using alternative measurement techniques. Methods for performing such measurements are also described in this disclosure.

As described in greater detail below, the measurement devices described herein allow electrical, electrochemical, and optical measurements to be performed on a cell culture microplate. The surface of the microplate can be physically divided into separate wells, with experiments run in parallel among the wells simultaneously. The number of electrodes that measure cellular responses in each well can be selected by partitioning the surface of the microplate, with the number of electrodes increasing as the size of wells increases. The measurement devices described herein include an electrical interface, allowing multiple devices to be connected to a common backplane. In this manner, measurement information from multiple measurement devices—each of which may include tens or hundreds of wells—can be acquired in parallel. Further, control information for experiments and measurements performed by each of the electrodes in each of the wells can be transmitted from the backplane to individual measurement devices.

In general, the measurement devices described herein include a plurality of complementary metal-oxide semiconductor (CMOS) integrated circuit (IC) chips on a common substrate. By using CMOS fabrication techniques to fabricate the ICs, several advantages can be realized. In some embodiments, sub-micrometer CMOS fabrication processes can be used to form integrated circuits that include dense arrays of electrodes. For example, electrode-to-electrode spacings in such arrays can be 25 micrometers or less, which allows for single cell measurements to be performed. That is, electrical and electrochemical properties of single cells can be measured using electrodes of the arrays. By grouping electrodes within wells on a semiconductor substrate (such that single wells contain many electrodes), spatially-resolved measurements can be performed to allow full well population statistical information to be obtained.

In certain embodiments, on or within the semiconductor substrate, electronic devices and components that are used for electrical and electrochemical measurements can be co-located with the electrodes of the integrated circuits. As such, each integrated circuit—including its electrodes and other electronic components—can function as an independent measurement system, capable of generating a variety of signals for stimulating cells through the electrodes, and measuring responses of the cells to the stimulation signals. Each integrated circuit can receive control signals (e.g., from a host controller) that cause the integrated circuit to perform these functions. Conventional measurement systems may rely on external electronic devices and circuitry, coupled to electrodes, to measure cellular responses. By integrating components in or on the semiconductor substrate, measurement systems are more compact and less costly, and multiple measurement devices can be operated in parallel, with measurement data transmitted to a common host controller.

### I. Overview

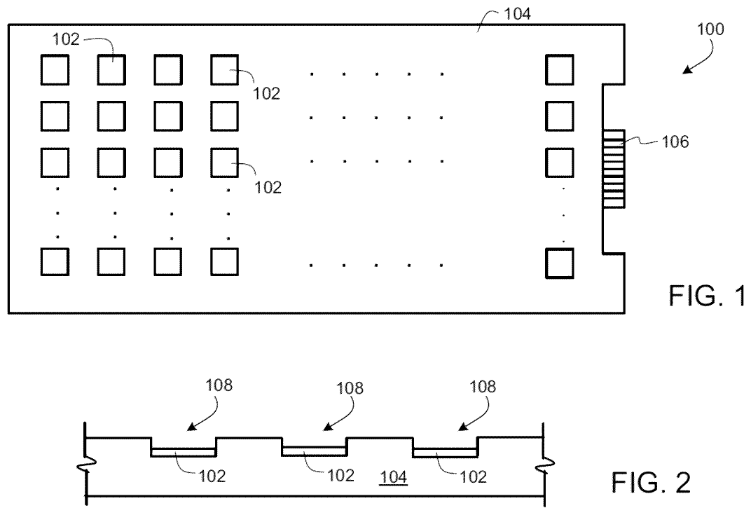

FIG. 1 is a schematic diagram of an example of measurement device 100. Device 100 includes a plurality of integrated circuits 102 fabricated on a substrate 104. Device 100 also includes an external connector 106 to which each of the integrated circuits 102 is connected. Connector 106 allows an external controller to transmit and receive signals from each of the integrated circuits 102.

Substrate 104 can be formed of any of a variety of materials. In some embodiments, for example, substrate 104 is printed circuit board formed of one or more plastic materials. In certain embodiments, substrate 104 includes one or more semiconducting materials. Connector 106 can be implemented in various ways. In some embodiments, for example, connector 106 is a finger-style connector with a plurality of exposed electrodes, and is configured to be received by a corresponding connector of another device. In certain embodiments, connector 106 includes a plurality of electrodes within a housing (not shown in FIG. 1), with the housing dimensioned to receive a mating connector of another device. More generally, connector 106 can be implemented in many different configurations that allow for electrical contact between device 100 and an external device.

On or within device 100, a plurality of connection lines (e.g., electrical traces) connect each of integrated circuits 102 to connector 106 (connection lines not shown in FIG. 1 for clarity). As will be discussed in more detail later, a host controller connected to device 100 through connector 106 can transmit electrical signals directly to any one or more of integrated circuits 102 via the connection lines.

In general, device 100 includes R rows of integrated circuits 102 and C columns of integrated circuits 102. R can generally be selected as desired based on the number of measurements and number of samples that are targeted for analysis. In some embodiments, for example, R can be 2 or more (e.g., 4 or more, 6 or more, 10 or more, 20 or more, 30 or more, 50 or more, 100 or more, 150 or more, 200 or more, 500 or more, or even more). Similarly, C can generally be selected as desired based on the number of measurements and number of samples that are targeted for analysis. In certain embodiments, C can be 2 or more (e.g., 4 or more, 6 or more, 10 or more, 20 or more, 30 or more, 50 or more, 100 or more, 150 or more, 200 or more, 500 or more, or even more).

In some embodiments, the number of rows R and columns C of integrated circuits 102 is selected to correspond to a standard well plate configuration. For example, in FIG. 1, device 100 can include 8 rows and 12 columns of integrated circuits 102, providing a total of 96 integrated circuits on substrate 104. The 8×12 geometry, with 96 total integrated circuits, matches a standard 96-well plate as is commonly used in the field of molecular biology.

In FIG. 1, integrated circuits 102 are positioned on substrate 104 in a regular array, with a common spacing between adjacent integrated circuits 102 along both rows and columns. In certain embodiments, integrated circuits 102 can be positioned in a regular array such that the spacing between adjacent integrated circuits along each row is the same, and the spacing between adjacent integrated circuits along each column is the same but different from the circuit-to-circuit spacing along each row. In some embodiments, the spacing between adjacent integrated circuits along each row is different in some rows. In some embodiments, the spacing between adjacent integrated circuits along each column is different in some columns.

While integrated circuits 102 in FIG. 1 are shown arranged in a regular square or rectangular array, more generally, other regular arrangements of integrated circuits 102 on substrate 104 can also be used. In certain embodiments, for example, integrated circuits 102 can be arranged along radial lines extending from a common center point, in a hexagonal array, in a pentagonal array, or more generally, in any regular arrangement on substrate 104.

In some embodiments, integrated circuits 102 can be positioned on substrate 104 in a non-regular and/or non-repeating arrangement. Methods for delivering electrical signals to cells and measuring cellular responses described herein generally do not require that such signals be delivered and responses measured from a regular arrangement of integrated circuits. As such, measurement devices can include non-regular arrangements of integrated circuits 102 on substrate 104, as well as any combination of one or more regular arrangements of integrated circuits 102 and one or more non-regular arrangements of integrated circuits 102.

In any of the foregoing arrangements of integrated circuits 102, a spacing between adjacent integrated circuits (e.g., along a row, along a column, or in another direction) can be selected as desired to control the density of measurement sites on substrate 104. For example, the spacing between adjacent circuits can be 3 mm or more (e.g., 4 mm or more, 5 mm or more, 6 mm or more, 7 mm or more, 8 mm or more, 9 mm or more, 10 mm or more, 12 mm or more, 15 mm or more, 20 mm or more, 25 mm or more, or even more). As an example, in some embodiments, device 100 includes an 8×12 array of integrated circuits 102 on substrate 104, with each integrated circuit spaced from adjacent integrated circuits along both rows and columns by 9 mm. With this spacing, the integrated circuits 102 of device 100 are located at positions on substrate 104 that match the positions of the wells on a standard 96-well plate as conventionally used in molecular biology.

Device 100 can be used to measure electrically induced responses under a variety of different conditions from one or more biological samples. In some embodiments, investigations are conducted in parallel on multiple biological samples. For example, each of the integrated circuits 102 shown in FIG. 1 can be configured to measure electrical and electrochemical responses from one or more different biological samples. Each such sample can include one or more cells.

To undertake measurements in parallel, integrated circuits 102 of device 100 can be positioned in individual recesses or wells. Each recess or well includes a wall that forms a barrier enclosing one or more components of the integrated circuit, preventing cross-contamination with other wells. For example, individual biological samples, along with optional chemical reagents, solvents, and other substances can be introduced into each well; the walls of each well prevent portions of the sample, reagents, solvents, and other substances from migrating to other wells on device 100.

Deploying integrated circuits 102 in wells can be implemented in a variety of ways. In some embodiments, integrated circuits 102 (and/or certain components of such circuits, such as electrodes) can be fabricated in recesses formed in substrate 104. FIG. 2 is a schematic diagram showing a cross-sectional view of an example of a substrate 104 that includes recesses 108. Integrated circuits 102 (or portions thereof) have been fabricated in each of the recesses. The recesses 108 are sufficiently deep such that biological samples, chemical reagents, and other substances can be introduced into each of the recesses 108, such that the integrated circuits 102 associated with each of the recesses 108 can selectively measure electrical and electrochemical information for samples deposited in the associated recesses 108.

In FIG. 2, the depth of each recess 108 is limited by the thickness of substrate 104, and the arrangement and number of wells is fixed by the positions of recesses 108 formed in substrate 104. As an alternative to fabricating integrated circuits 102 (or portions thereof) in recesses, in some embodiments the integrated circuits 102 can first be fabricated on substrate 104, after which wells can be formed around the integrated circuits 102, with each integrated circuit 102 enclosed by walls.

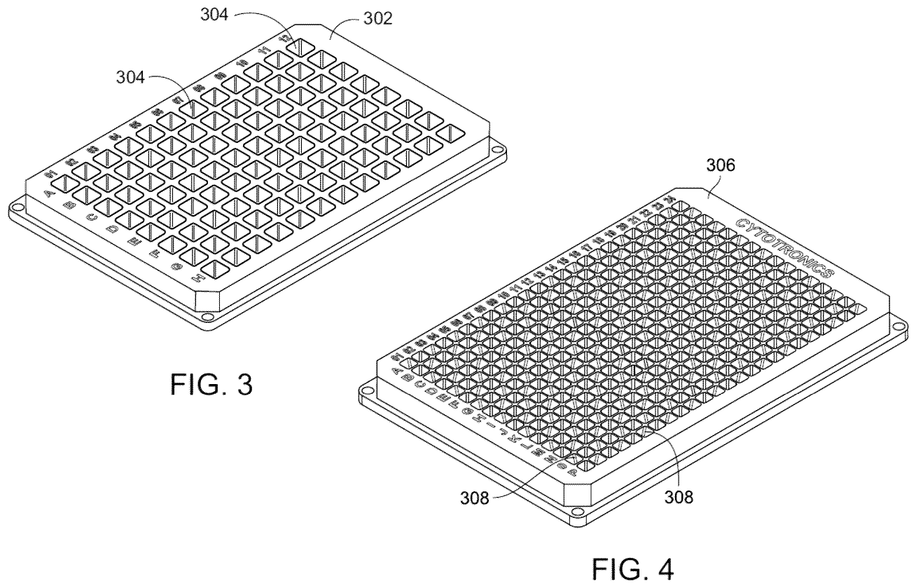

To form wells in such a manner, an enclosure 302 can be overlaid atop substrate 104. FIG. 3 is a schematic diagram showing an enclosure that can be overlaid on top of a substrate 104 on which a plurality of integrated circuits 102 have been fabricated. Enclosure 302 includes a plurality of apertures 304 that extend through the enclosure. Apertures 304 are positioned within enclosure 302 such that they are aligned with the integrated circuits 102 formed on substrate 104. Accordingly, when enclosure 302 is fixed to substrate 104, the walls of each of the apertures 304 enclose one of the integrated circuits 102, forming a well with integrated circuit 102 positioned at the bottom of the well. A biological sample can be introduced into each well formed in this manner, along with chemical reagents, solvents, and other compounds for performing a variety of electrical and electrochemical measurements.

In general, a liquid- and gas-impermeable seal is formed between enclosure 302 and substrate 104, so that leakage does not occur through the bottom of the wells (i.e., between enclosure 302 and substrate 104). To form such a seal, substrate 104 can be directly bonded using epoxy to enclosure 302, isolating each of the volumes contained within each well.

In some embodiments, enclosure 302 can be bonded to substrate 104 using a biocompatible rigid epoxy or flexible silicone material. For example, a flexible silicone such as MasterSil 153Med (available from Masterbond, Inc., Hackensack, NJ) can be used to bond enclosure 302 to substrate 104. In some embodiments, the underside of enclosure 302 (i.e., the surface that contacts substrate 104) includes routed channels, and the channels can be backfilled with epoxy to further affix enclosure 302 to substrate 104 and provide structural support to the measurement device.

In certain embodiments, enclosure 302 can be secured to substrate 104 using a mechanical sealing mechanism. For example, a gasket can be positioned between substrate 104 and enclosure 302, and a mechanical clamping mechanism can be used to apply pressure to substrate 104 and enclosure 302, sandwiching the gasket and sealing the interstitial space between substrate 104 and enclosure 302. In some embodiments, the gasket (not shown in FIG. 3) is formed from a water-impermeable, biocompatible material that is at least partially compressible. When pressure is applied, the gasket deforms to form a water-impermeable barrier ensuring that fluid does not leak out of the interstitial space between substrate 104 and enclosure 302. A variety of different materials can be used to form the gasket including, but not limited to, biocompatible thermoplastic materials. In certain embodiments, the material(s) from which the gasket is formed do not bond chemically to either substrate 104 or enclosure 302, and consequently, the gasket can be removed to facilitate cleaning of device 100 and/or enclosure 302.

A variety of different mechanical sealing mechanisms can be used to secure enclosure 302 to substrate 104 with the gasket in between. In some embodiments, the mechanical sealing mechanism includes one or more screws or other threaded members that engage with counterpart threaded members and can be selectively adjusted to control the amount of pressure applied to substrate 104 and/or enclosure 302. In certain embodiments, the mechanical sealing mechanism includes a latch that can be engaged with either or both of enclosure 302 and substrate 104. The latch can be permanently attached to substrate 104, to enclosure 302, or to neither substrate 104 nor enclosure 302. In some embodiments, the mechanical sealing mechanism includes a member with an internal channel dimensioned to receive and engage with the edges of substrate 104 and enclosure 302. The mechanical sealing mechanism can be configured to be positioned around at least a portion of a perimeter of substrate 104 and/or enclosure 302, forming a frame that holds substrate 104 and enclosure 302 together. The member can be formed from a rigid material (e.g., one or more metals or rigid plastic materials) or, alternatively, can be formed from a compliant material that can be deformed to facilitate securing substrate 104 and enclosure 302.

It should further be noted that the foregoing are merely examples of suitable mechanical sealing mechanisms, and more generally, any mechanism that secures substrate 104 to enclosure 302 can be used in measurement device 100.

Returning to FIG. 3, a 1:1 relationship exists between the number of wells formed and the number of integrated circuits 102, with one integrated circuit positioned within each well. However, it is not a requirement that individual wells have an entire dedicated integrated circuit. To the contrary, in some embodiments, a single integrated circuit can be used to make measurements in multiple wells by forming wells in such a manner that they effectively sub-divide portions of the surface of substrate 104 where integrated circuits 102 are formed. Because each integrated circuit 102 includes a large number of electrodes, groups of electrodes can be allocated to measurements in different wells.

In general, single integrated circuits 102 can be used to make measurements in one or more (e.g., two or more, three or more, four or more, eight or more, 12 or more, 16 or more, 32 or more, 64 or more, or even more) wells by partitioning the active area of the integrated circuit (that is, the spatial distribution of electrodes of the integrated circuit at the surface of substrate 104) using well walls, such that different groups of electrodes of an integrated circuit are positioned within different wells. In effect, the electrodes of any integrated circuit can be partitioned any number of ways and dedicated to measurements in different wells.

FIG. 4 shows a schematic diagram of an example of an enclosure 306 in which each of the wells of enclosure 302 in FIG. 3 has been further partitioned into 4 wells 308 of equal size. The alignment of the wells in enclosure 306 is the same as in enclosure 302, so that when enclosure 306 is positioned relative to substrate 104 as described above, the active area of each integrated circuit 102 is partitioned into 4 equal regions. Thus, each integrated circuit 102 is used to perform measurements in 4 different wells in the resulting measurement device. As an example, for a measurement device that includes an 8×12 array of integrated circuits 102, enclosure 302 forms a device that includes 96 distinct wells with one integrated circuit dedicated to each well, while enclosure 306 forms a device that includes 384 wells with one integrated circuit dedicated to a group of 4 wells. In some embodiments, the principle shown in FIG. 4 can be extended to an enclosure which divides each of the wells of enclosure 302 into 16 distinct wells, with one integrated circuit dedicated to each group of 16 wells. Such a device has a total of 1536 wells.

In the foregoing examples, the walls of wells are used to sub-divide the active area of each integrated circuit into equal regions, each with the same number of electrodes. It should be understood however that the active area of individual integrated circuits can be partitioned equally or unequally, with the same number or different numbers of electrodes of an integrated circuit dedicated (i.e., positioned within) different wells. Further, while the wells shown in FIGS. 3 and 4 have a generally square cross-sectional shape, more generally the wells of the measurement devices described herein can have any cross-sectional shape, and a single measurement device can have wells of more than one shape formed on the device. Additional well cross-sectional shapes that can be used include, but are not limited to, rectangular, circular, elliptical, pentagonal, hexagonal, octagonal, and more generally, any regular or irregular cross-sectional shape.

In general, enclosure 302 can be formed from a variety of materials. For example, in some embodiments, enclosure 302 is formed from one or more biocompatible polymers such as, but not limited to, polystyrenes, polycarbonates, and polyethylene terephthalates. In certain embodiments, enclosure 302 is formed from one or more other plastics, silicones, and/or polymers.

The number of electrodes of an integrated circuit positioned within each well of a measurement device depends on the density of the electrodes, the lateral width of the walls of the well, and the number of wells formed on the measurement device, among other factors. In general, the number of electrodes of an integrated circuit positioned within each well can be 1 or more (e.g., 10 or more, 100 or more, 1000 or more, 5000 or more, 10,000 or more, 50,000 or more, 100,000 or more, 200,000 or more, 500,000 or more, 1×10⁶ or more, or even more).

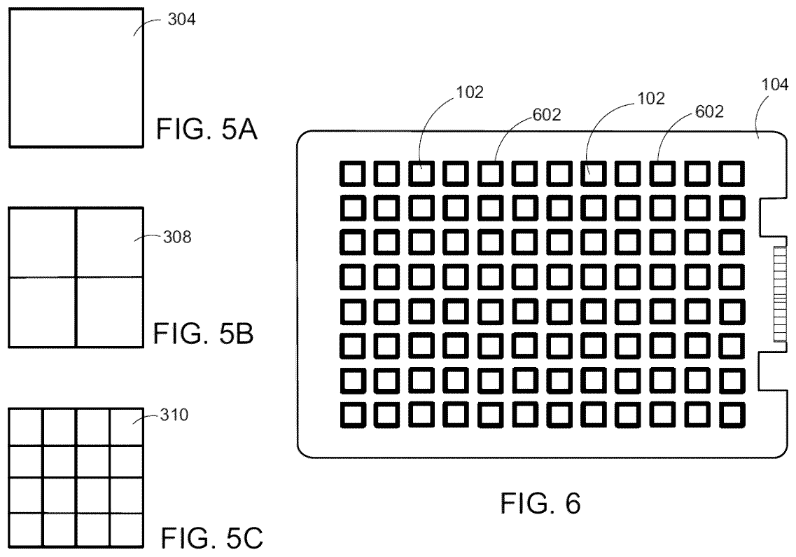

To understand how electrodes of an integrated circuit are dedicated to individual wells within a measurement device, it is instructive to review an example. Consider, for example, an integrated circuit that includes 384×384 electrodes arranged in a square array, with an electrode pitch (i.e., an electrode-to-electrode spacing) of 12.5 microns. For a measurement device in which the entire active area (e.g., the entire electrode array) of the integrated circuit is positioned within a single well (e.g., as occurs with the enclosure of FIG. 3), a total of 147,456 electrodes are dedicated to measurements within the well. If the array of electrodes extends for a distance of 4.8 mm along each dimension of the array, then the active area of the well is 4.8 mm×4.8 mm. FIG. 5A is a schematic diagram showing a well 304 with these dimensions.

However, for a measurement device in which the active area of the integrated circuit is sub-divided into 4 wells (e.g., as occurs with the enclosure of FIG. 4), then the active area of each well is reduced. FIG. 5B is a schematic diagram showing an example of the active area of an integrated circuit that is sub-divided among four wells 308. The internal walls of the wells overlie portions of the integrated circuit, and even if no electrodes are obscured, the active area of each well is reduced. When the active area of the above integrated circuit is sub-divided into 4 regions (with each region dedicated to a different well), the active area of each well may be approximately 2.2 mm×2.2 mm, with a total of 36,864 electrodes dedicated to each well, assuming that the walls of the wells have a thickness of 400 microns. Similarly, FIG. 5C shows a schematic diagram of the active area of an integrated circuit that is sub-divided among 16 wells. Under these circumstances, the active area of each well is 1.0 mm×1.0 mm, with a total of 9,216 electrodes positioned within each well.

In FIGS. 3 and 4, an enclosure 302/306 was positioned relative to substrate 104 to form wells on the measurement device. However, other methods can also be used to form wells that surround (and, in some embodiments, sub-divide the active area of) integrated circuits on substrate 104. In certain embodiments, for example, pre-formed individual well structures can be bonded individually or in groups to substrate 104. FIG. 6 is a schematic diagram showing a substrate 104 that includes a plurality of integrated circuits 102, each of which is surrounded by a well structure 602 that is bonded to substrate 104. Bonding can be achieved using epoxy for example, in a manner similar to the bonding between enclosures 302/306 and substrate 104.

Well structures 602 can be formed from a variety of materials including, but not limited to, polystyrenes, polycarbonates, polyethylene terephthalates, other plastics, silicone-based materials, and various fluorinated polymers. The cross-sectional shapes of well structures 602 can include any of the shapes described above in connection with enclosures 302/306, and can, in general, be any regular or irregular shape. Well structures 602 can all be the same size and/or cross-sectional shape, or alternatively, well structures of different sizes and/or shapes can be bonded to substrate 104. In addition to well structures that individually enclose a single integrated circuit as shown in FIG. 6, well structures 602 can have internal walls that sub-divide the active area of integrated circuits among a plurality of wells (as shown for example in FIG. 4).

In certain embodiments, well structures similar to well structures 602 can be formed directly on the surface of substrate 104 by direct injection molding of polystyrenes, polycarbonates, polyethylene terephthalates, other plastics, silicones, and other materials. Well structures formed in this manner can have any of the properties discussed above in connection with the wells of enclosures 302/306 and well structures 602.

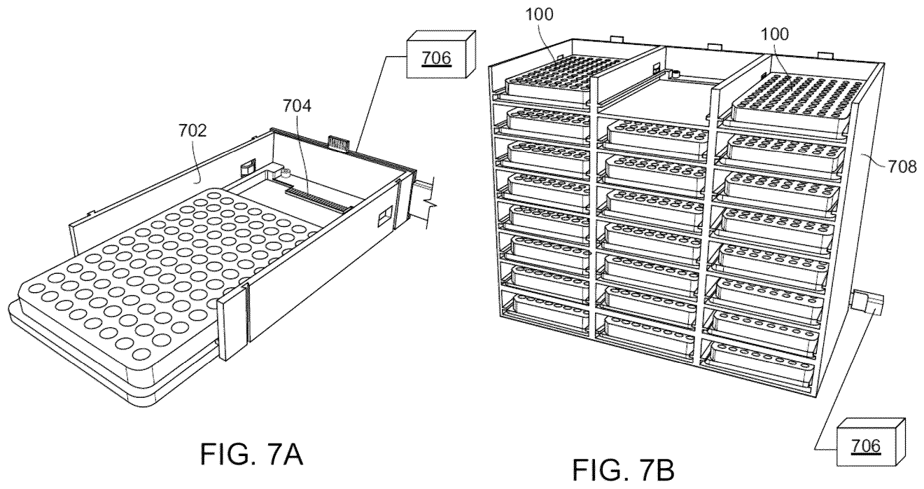

In some embodiments, the measurement devices described herein are dimensioned to be received in a housing to facilitate connection to an external host controller, which can transmit control signals and receive measurement signals from the measurement devices. An example of such a housing is shown in the perspective view of FIG. 7A. Housing 702 is dimensioned to receive measurement device 100. When measurement device 100 is inserted into the housing, connector 106 engages with interface 704 of housing 702. Interface 704 is configured to connect to a host controller 706, which can manage some or all aspects and steps of configuration of measurement device 100 and acquisition of measurement information. Housing 702 functions as a stand-alone unit that can be used to control and acquire data from each of the integrated circuits 102 of measurement device 100. Control signals and measurement information are transmitted between integrated circuits 102 of measurement device 100 and host controller 706 through connector 106 and interface 704.

Multiple housings 702 can be stacked together to generate a housing array 708 that can receive and control multiple measurement devices 100, as shown in the perspective view of FIG. 7B. Housing array 708 allows host controller 706 to be connected to multiple measurement devices, to control each of the measurement devices, and to obtain measurement information from each of the measurement devices serially or in parallel. The multiplex advantage arising from performing measurements in parallel enables thousands of different experiments to be conducted on biological samples simultaneously.

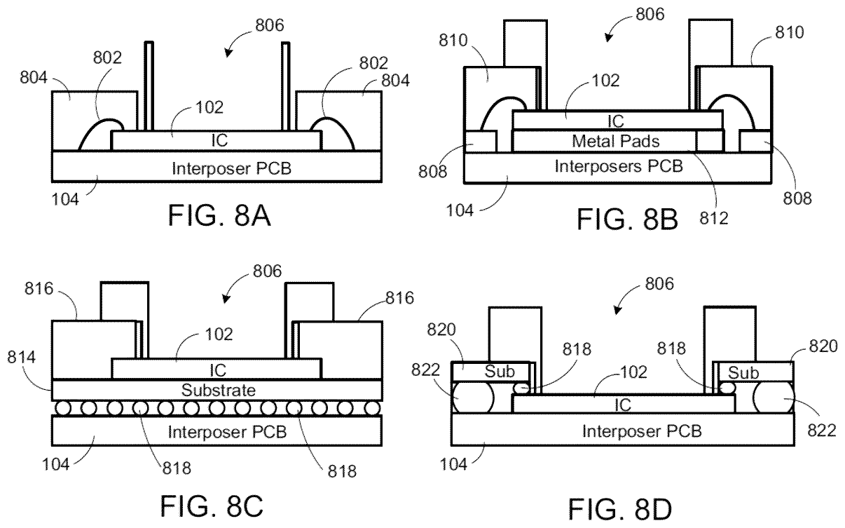

Returning to FIG. 1, as discussed above, the integrated circuits 102 can be formed using CMOS fabrication methods, permitting dense arrays of electrodes to be fabricated within each integrated circuit chip. After integrated circuit chips have been fabricated, the chips can be attached to substrate 104 using a variety of different methods. In some embodiments, for example, a chip-on-board (COB) method is used to attach the integrated circuit chips. FIG. 8A is a schematic diagram showing attachment of an integrated circuit (IC) chip 102 to substrate 104. IC chip 102 is placed directly in contact with substrate 104 and wirebonded to substrate 104 via conductors 802. The wirebonding conductors 802 are then overmolded with an epoxy encapsulant 804. One or more wells 806 surrounding chip 102 (or portions thereof) can be formed using any of the methods described above.

COB packaging can present a number of challenges. Wirebonding IC chip 102 and then overmolding with encapsulant 804 are processes that can, in some circumstances, be difficult to automate. Functional IC testing can sometimes be difficult, and if a single IC chip is improperly bonded to substrate 104, it can be difficult to correct the error.

Accordingly, in certain embodiments, after IC chips 102 have been fabricated, they are attached to substrate 104 using an open cavity quad flat no-lead (QFN) packaging method. FIG. 8B is a schematic diagram showing an example of the open cavity QFN packaging method. IC chip 102 is wirebonded to a metal leadframe 808, and the wirebonds are encapsulated in an epoxy layer to protect the wirebonds and form a rigid package. Well 806 surrounding IC chip 102 (or a portion thereof) can be formed using any of the methods described above. A solder layer 812 can then be directly flowed to bond IC chip 102 to substrate 104.

Leadframe 808 improves the thermal conductivity of the device, allowing heat generated during operation of IC chip 102 to be dissipated through substrate 104. Dissipation of excess heat energy allows for improved control of temperature changes in the biological sample during operation of the measurement device.

The QFN packaging method can be used to realize certain advantages relative to the COB method. Individual IC chips 102 can be tested before permanent attachment to substrate 104, and defective chips can be replaced before attachment, leading to lower failure rates for the measurement device.

Another method for fixing IC chips 102 to substrate 104 is the ball grid array method. FIG. 8C is a schematic diagram showing an example of this method. In this method, IC chip 102 is wirebonded to a substrate 814, and the wirebonds are encapsulated in an epoxy 816. The bottom of substrate 814 includes a grid array of solder balls 818 that are routed to the wirebonds. Well 806 surrounding IC chip 102 (or a portion thereof) can be formed using any of the methods described above.

In some embodiments, heat dissipation when using the ball grid array method to fix IC chips 102 to substrate 14 can be compromised relative to the QFN packaging method, due to the reduced area of contact between substrate 814 and substrate 104. Proper thermal monitoring and management to avoid conducting excess heat into a biological sample within well 806 is a feature of such implementations.

A further method for fixing IC chips 102 to substrate 104 is the flip-chip ball grid array (FCBGA) method. FIG. 8D is a schematic diagram that shows an example of this method. Small diameter solder balls 818 positioned on wirebond pads of IC chip 102 are used to connect IC chip 102 to a substrate 820. Positioned on substrate 820 are larger solder balls 822 that connect substrate 820 to substrate 104. Well 806 surrounding IC chip 102 (or a portion thereof) can be formed using any of the methods described above.

The smaller solder balls 818 eliminate wirebonding of IC chip 102 to substrate 820, which allows for a larger active area of IC chip 102, and for a larger well 806 to be formed around IC chip 102. The absence of wirebonds also improves the robustness of the chip package. IC chip 102 is also in direct contact with substrate 104 due to the flip-chip nature of the method, creating an efficient path for the dissipation of thermal energy from IC chip 102 to substrate 104.

### II. Integrated Circuits for Measurement Devices

As discussed above, each of the integrated circuits 102 of measurement device 100 functions as an independent measurement component, and is configurable, upon receipt of suitable control instructions (e.g., from host controller 706), to perform a wide variety of functions, including generating electrical signals to be applied to biological samples, and measuring information (e.g., electrical and electrochemical parameters) of the biological samples in response to the applied signals. In this section, a variety of aspects of the integrated circuits 102 will be discussed.

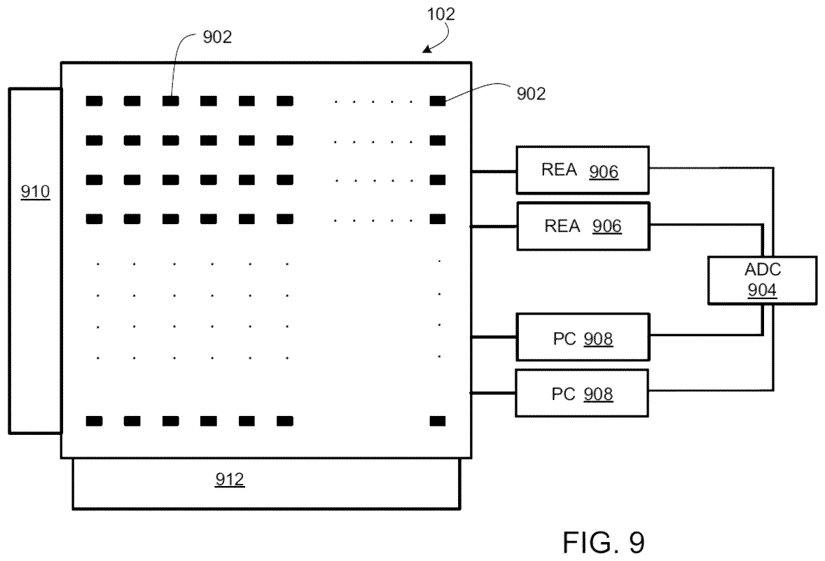

FIG. 9 is a schematic diagram of an example of an integrated circuit 102. Integrated circuit 102 includes a plurality of electrodes 902. Electrodes 902 are positioned in a regular, two-dimensional array in FIG. 9. Integrated circuit 102 includes one or more reference electrode amplifiers (REAs) 906 and one or more peripheral circuits (PCs) 908. The REAs 906 and PCs 908 can each selectively couple to one or more electrodes 902. In addition, integrated circuit 102 includes one or more analog-to-digital converters (ADCs) 904, each of which is coupled to (and receives measurement signals from) one or more REAs 906 and/or one or more PCs 908.

As shown in FIG. 9, integrated circuit 102 also includes a row select logic unit 910 and a column select logic unit 912. These logic units function to selectively enable particular rows and columns of electrodes 902. For example, the logic units can transmit control signals to particular rows and/or columns of electrodes 902 to prepare the electrodes in those rows and columns to receive configuration signals (e.g., from host controller 706 and/or from a memory unit that stores configuration bits for the electrodes). By enabling rows and/or columns of electrodes in this manner, individual electrodes 902 can be selectively configured, e.g., to perform particular measurements.

It should be noted that, although not shown in FIG. 9, certain components of integrated circuit 102 are also coupled to connector 106, and therefore can connect to host controller 706. The connected components can receive control signals from host controller 706 that cause the components to perform a variety of functions, as described in greater detail below. The connected components can also transmit signals that include, e.g., measurement information, to host controller 706.

As shown in FIG. 9, each of the electrodes 902 is spaced from adjacent electrodes. In general, the spacings Δx and Δy can be selected as desired to achieve an electrode array with a particular spatial resolution, depending on factors such as a desired measurement resolution, the nature of the sample being measured, and the desired field geometry. The spacings Δx and Δy can each be independently selected, and can be 1 mm or less (e.g., 800 micrometers or less, 600 micrometers or less, 500 micrometers or less, 400 micrometers or less, 300 micrometers or less, 200 micrometers or less, 100 micrometers or less, 75 micrometers or less, 50 micrometers or less, 40 micrometers or less, 30 micrometers or less, 25 micrometers or less, 20 micrometers or less, 15 micrometers or less, 10 micrometers or less, 5 micrometers or less, 3 micrometers or less, or even less). In some embodiments, each of the spacings Δx and Δy is independently selected, and is between 5 micrometers and 1 mm (e.g., between 5 micrometers and 500 micrometers, between 10 micrometers and 500 micrometers, between 10 micrometers and 400 micrometers, between 15 micrometers and 400 micrometers, between 15 micrometers and 300 micrometers, between 20 micrometers and 300 micrometers, between 20 micrometers and 200 micrometers, between 25 micrometers and 200 micrometers, between 25 micrometers and 100 micrometers, or any range within any of the foregoing ranges).

In FIG. 9, electrodes 902 are arranged in a square or rectangular array with Δx and Δy being the same or different. More generally, electrodes can be arranged in a variety of different array geometries. For example, electrodes 902 can be arranged in a square array, a rectangular array, a hexagonal array, an octagonal array, an annular array, a spiral array, a radial array, or any other type of array defined by a regular, repeating geometric pattern of electrodes. Alternatively, in some embodiments, electrodes 902 can be positioned such that no repeating, spatial ordering of the electrodes 902 is defined by their arrangement, i.e., in an irregular spatial arrangement.

In FIG. 9, the electrode-to-electrode spacings Δx and Δy are measured in orthogonal directions within the array. More generally, however, the spacings can represent nearest-neighbor distances between electrodes that are measured along non-orthogonal directions.

In some embodiments, all of the electrodes 902 are arranged in an array defined by a common repeating spatial pattern of electrodes. In certain embodiments, some of the electrodes 902 can be arranged in a first array defined by a first pattern of electrodes, and some of the electrodes 902 can be arranged in a second array defined by a second pattern of electrodes. As one example, the first array can be a square or rectangular array, and the second array can be a hexagonal array. More generally, any combination of array types—including (but not limited to) the types described above—can be present in integrated circuit 102.

The two arrays of electrodes can, in some embodiments, be spatially separated from one another in integrated circuit 102. Alternatively, in certain embodiments, the first and second arrays can be at least partially interleaved, such that at least some electrodes of the second array are located spatially within the boundary of the first array, and vice versa. In some embodiments, the arrays can be fully interleaved, e.g., with one of the arrays located entirely within the boundary of the other array.

Although the foregoing discussion focuses on two arrays of electrodes, more generally the electrodes 902 of integrated circuit 902 can be arranged in any number (e.g., 2 or more, 3 or more, 4 or more, 5 or more, 6 or more, 7 or more, 8 or more, 10 or more, 12 or more, 16 or more, 32 or more, or even more) of arrays, each of which can have any of the properties described above.

In general, the number of electrodes 902 within integrated circuit 102 can be selected based on criteria such as the number of samples to be measured, the desired spatial resolution of measurements, power consumption considerations, and measurement frequency. In some embodiments, for example, integrated circuit 102 includes at least 3000 electrodes (e.g., at least 4000 electrodes, at least 5000 electrodes, at least 10,000 electrodes, at least 20,000 electrodes, at least 30,000 electrodes, at least 50,000 electrodes, at least 75,000 electrodes, at least 1.0×10¹ electrodes, at least 1.5×10¹ electrodes, at least 2.0×10¹ electrodes, at least 2.5×10¹ electrodes, at least 3.0×10¹ electrodes, at least 5.0×10¹ electrodes (e.g., at least 7.5×10¹ electrodes, at least 1.0×10⁶ electrodes, at least 3.0×10⁶ electrodes, at least 5.0×10⁶ electrodes, at least 7.0×10⁶ electrodes, at least 1.0×10⁷ electrodes, at least 1.2×10⁷ electrodes, at least 1.4×10⁷ electrodes, at least 1.6×10⁷ electrodes, at least 1.8×10⁷ electrodes, at least 2.0×10⁷ electrodes, or even more electrodes).

The active area of integrated circuit 102, which corresponds to the on-chip area occupied by the array of electrodes 902, generally depends on the electrode-to-electrode spacing and the total number of electrodes. In certain embodiments, for example, the active area of integrated circuit 102 is 0.5 mm² (e.g., 1.0 mm², 2.0 mm², 3.0 mm², 5.0 mm², 6.0 mm², 8.0 mm², 10.0 mm², 12.0 mm², 14.0 mm², 16.0 mm², 18.0 mm², 20.0 mm², 25.0 mm², 30.0 mm², 50.0 mm², 1.0×10² mm², 1.5×10² mm², 2.0×10² mm², 3.0×mm², 4.0×10² mm², 5.0×10² mm², 6.0×10² mm², 7.0×10² mm², 8.0×10² mm², 9.0×10² mm², 1.0×10³ mm² or more (e.g., 1.2×10³ mm² or more, 1.4×10³ mm² or more, 1.6×10³ mm² or more, 1.8×10³ mm² or more, 2.0×10³ mm² or more, 2.2×10³ mm² or more, 2.4×10³ mm² or more, 2.6×10³ mm² or more, 2.8×10³ mm² or more, 3.0×10³ mm² or more, or even more).

In the following discussion, the example shown in FIG. 9—in which electrodes 902 are arranged in a spatially uniform array of consistent electrode spacing—will be discussed for purposes of clarity. However, it should be appreciated that the additional features of electrodes 902 described below apply to electrodes positioned in any of the arrangements described above, unless expressly stated otherwise.

### (a) Pixel Circuits

Each electrode 902 of integrated circuit 102 can generally be connected to a variety of different components and circuit elements. In general, each electrode 902 is connected to a pixel circuit that includes a variety of different components which are configurable, in response to one or more control signals, to control the functions performed by electrode 902. A wide variety of different pixel circuits can be used in integrated circuit 102. In some embodiments, each electrode 902 is coupled to its own pixel circuit such that there is one dedicated pixel circuit for every electrode 902 in integrated circuit 102. In certain embodiments, one or more electrodes may share a pixel circuit, and can be individually coupled to the pixel circuit, or alternatively, groups of one or more electrodes can be simultaneously coupled to a shared pixel circuit.

In general, the pixel circuit to which an electrode 902 is coupled can perform at least two functions. First, the pixel circuit can deliver one or more electrical signals (e.g., voltage signals, current signals) to electrode 902. Second, the pixel circuit can receive one or more electrical signals at electrode 902. Depending upon the configuration of the pixel circuit, each of these functions can be implemented in different (and multiple) ways.

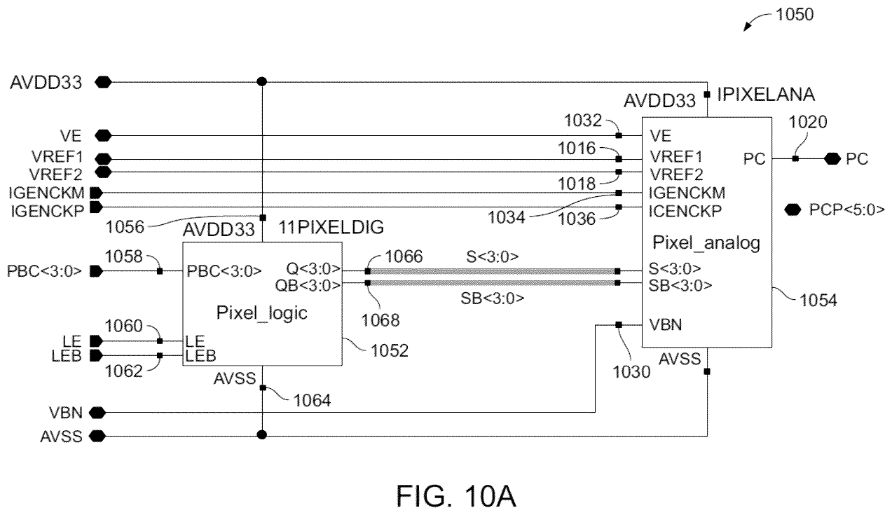

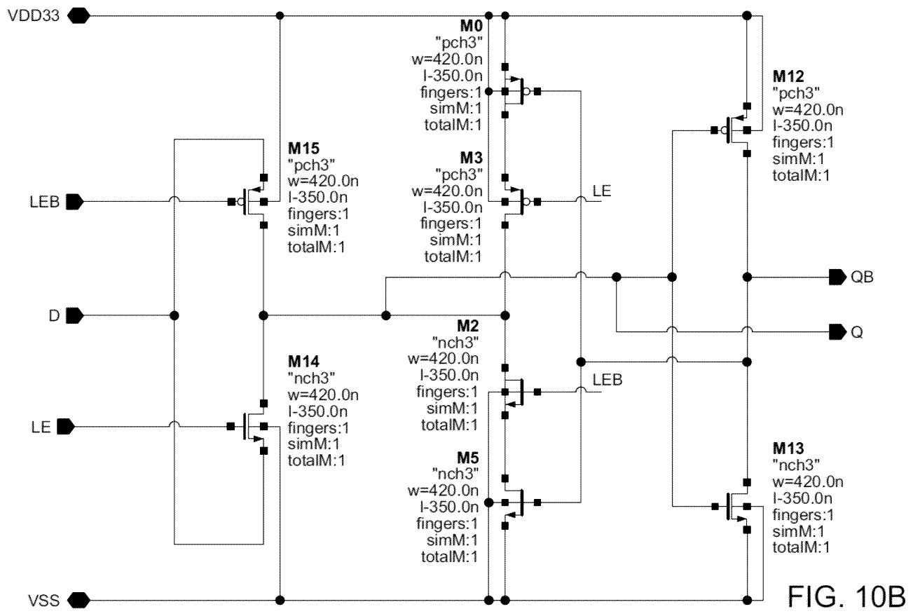

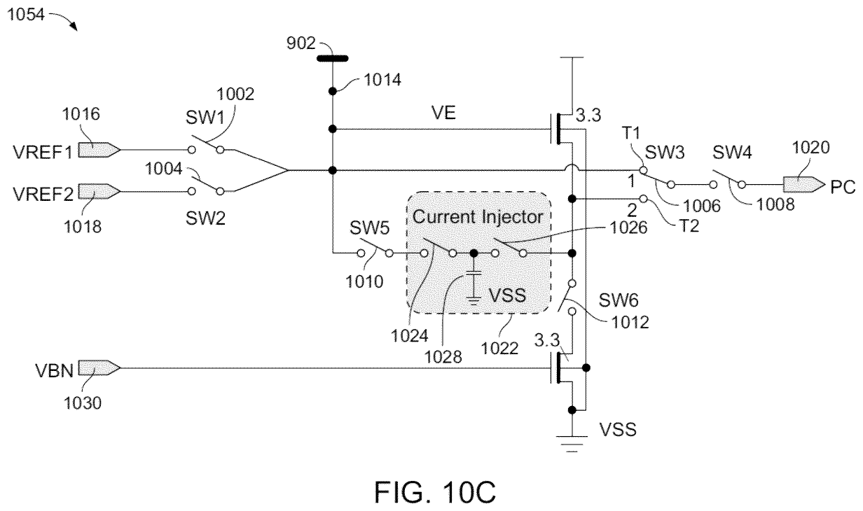

FIG. 10A is a schematic diagram of an example pixel circuit 1050 to which electrode 902 can be coupled. Pixel circuit 1050 includes a pixel logic block 1052 and a pixel analog circuit 1054. Not shown in FIG. 10A is a memory unit that can be used to store settings for switches in pixel analog circuit 1054. In general, pixel logic block 1052 controls the configuration of one or more switches in pixel analog circuit 1054, thereby selecting the functional attributes of pixel analog circuit 1054.

Pixel logic block 1052 includes input terminals 1056, 1058, 1060, 1062, and 1064, and output terminals 1066 and 1066. At terminal 1056, pixel logic block 1052 receives an input voltage signal from an analog voltage source AVDD33. Typically, the voltage signal has a magnitude of about 3.3 V, although voltages signals of other magnitudes can also be supplied.

At terminal 1058, pixel logic block 1052 receives configuration programming bit values, e.g., directly or indirectly from host controller 706, or from a memory unit that stores bit values for configuring electrode 902. At terminals 1060 and 1062, pixel logic block 1052 receives assertion and/or de-assertion signals from row select logic unit 910 and/or column select logic unit 920 that control the operation of latches within pixel logic block 1052. At output terminals 1066 and 1068, configuration bit values generated by pixel logic block 1052 are transmitted to pixel analog circuit 1054 to control the configuration of switches within pixel analog circuit 1054, as will be described in more detail below.

To control the configuration of switches in pixel analog circuit 1054, pixel logic block 1052 contains 4 D-latches. FIG. 10B is a schematic diagram showing an example of one of the D-latches. In FIG. 10B, the D-latch is transparent when its LE input is asserted and the LEB input is de-asserted via control signals received at terminals 1060 and 1062. Each D-latch generates corresponding output bit values QB and B, which are transmitted at terminals 1066 and 1068 of pixel logic block 1052. The output bit values of each of the 4 D-latches, Q<3:0> and QB<3:0>, are transmitted to pixel analog circuit 1054 as bit signals S<3:0> and SB<3:0> as shown in FIG. 10A.

FIG. 10C is a schematic diagram showing an example of a pixel analog circuit 1054. Pixel analog circuit 1054 includes six switches SW1 (1002), SW2 (1004), SW3 (1006), SW4 (1008), SW5 (1010), and SW6 (1012). Each of switches 1002, 1004, 1008, 1010, and 1012 is switchable between open and closed states. Switch 1006 is switchable between terminal T1 and terminal T2. Pixel analog circuit 1054 includes a terminal 1016 at which a reference voltage signal V_{REF1} can be received from a reference amplifier 906, from a peripheral circuit 908, or from a digital-to-analog converter (DAC), and a terminal 1018 at which a reference voltage signal V_{REF2} can optionally be received from a reference amplifier 906, from a peripheral circuit 908, or from a digital-to-analog converter (DAC). Pixel analog circuit 1054 also includes a terminal 1030 at which a bias voltage signal for the current source of the source follower portion of pixel analog circuit 1054 is received.

Referring to FIG. 10A, pixel analog circuit 1054 also includes other terminals that are not shown in FIG. 10C for purposes of clarity. Pixel analog circuit 1054 includes terminal 1032, at which pixel analog circuit 1054 receives a voltage bias signal for electrode 902 from a reference amplifier 906, from a peripheral circuit 908, or from a digital-to-analog converter (DAC). Pixel analog circuit 1054 includes terminals 1034 and 1036 at which clock signals IGENCKM and IGENCKP are received and used in certain operating modes to inject current via electrode 902, as discussed further below.

Returning to FIG. 10C, pixel analog circuit 1054 is coupled to electrode 902 at terminal 1014. Electrical signals (e.g., voltage signals, current signals) are delivered to electrode 902 through terminal 1014, and electrical signals at electrode 902 are measured at terminal 1014. Pixel analog circuit 1054 also includes an output terminal 1020 that is selectively connectable to a peripheral circuit 908 so that measurement signals detected by electrode 902 can be routed to a peripheral circuit 908 through a source follower portion of pixel analog circuit 1054.

Pixel analog circuit 1054 includes a current injector 1022 that includes a capacitor 1028 and switches 1024 and 1026. Switches 1024 and 1026 are connected to terminals 1034 and 1036 respectively, and when switches 1024 and 1026 receive suitable input signals via these terminals, current injector 1022 is “on” and injects current from capacitor 1028 at electrode 902. When switches 1024 and 1026 do not receive suitable signals via terminals 1034 and 1036, capacitor 1028 is isolated and current injector 1022 is “off”.

The different switches of pixel analog circuit 1054 in FIG. 10C allow each electrode 902 to be operated in a number of different modes. Table 1 summarizes 14 different modes in which electrode 902 can be operated, depending upon the state of switches SW1-SW6 and current injector 1022 in pixel analog circuit 1054.

TABLE 1

| Config | SW1 | SW2 | SW3 | SW4 | SW5 | SW6 | Curr Inj |
|---|---|---|---|---|---|---|---|
| 1 | Open | Open | T2 | Closed | Closed | Closed | On |
| 2 | Open | Open | T2 | Open | Closed | Closed | On |
| 3 | Open | Open | T2 | Closed | Closed | Closed | Off |
| 4 | Open | Open | T2 | Open | Closed | Closed | Off |
| 5 | Closed | Open | T2 | Closed | Closed | Closed | Off |
| 6 | Closed | Open | T2 | Open | Closed | Closed | Off |
| 7 | Open | Closed | T2 | Closed | Closed | Closed | Off |
| 8 | Open | Closed | T2 | Open | Closed | Closed | Off |
| 9 | Open | Open | T1 | Closed | Open | Open | Off |
| 10 | Closed | Open | T1 | Closed | Open | Open | Off |
| 11 | Closed | Open | T1 | Open | Open | Open | Off |
| 12 | Open | Closed | T1 | Closed | Open | Open | Off |
| 13 | Open | Closed | T1 | Open | Open | Open | Off |
| 14 | Open | Open | T1 | Open | Open | Open | Off |

In Table 1, logical controls for switches SW5, SW6, and SW3 can be shared since SW3 is always connected to T2 when switches SW5 and SW6 are closed, and connected to T1 when switches SW5 and SW6 are open. Accordingly, the number of storage elements used to configure an electrode can be reduced from 6 to 4 by logical control sharing, which simplifies the design of pixel circuit 1050 and reduces the area of electrode 902 in integrated circuit 102. By sharing logical controls for switches SW5, SW6, and SW3, the various operating modes shown in Table 1 can be represented by a truth table of 4 configuration bits PBC<3:0>, as shown in Table 2 below.

TABLE 2

| Config | PBC<0> | PBC<1> | PBC<2> | PBC<3> | IGENCKP | IGENCKM |
|---|---|---|---|---|---|---|
| 1 | 0 | 0 | 1 | 1 | Toggle | Toggle |
| 2 | 0 | 0 | 1 | 0 | Toggle | Toggle |
| 3 | 0 | 0 | 1 | 1 | 0 | 0 |
| 4 | 0 | 0 | 1 | 0 | 0 | 0 |
| 5 | 1 | 0 | 1 | 1 | 0 | 0 |
| 6 | 1 | 0 | 1 | 0 | 0 | 0 |
| 7 | 0 | 1 | 1 | 1 | 0 | 0 |
| 8 | 0 | 1 | 1 | 0 | 0 | 0 |
| 9 | 0 | 0 | 0 | 1 | 0 | 0 |
| 10 | 1 | 0 | 0 | 1 | 0 | 0 |
| 11 | 1 | 0 | 0 | 0 | 0 | 0 |
| 12 | 0 | 1 | 0 | 1 | 0 | 0 |
| 13 | 0 | 1 | 0 | 0 | 0 | 0 |
| 14 | 0 | 0 | 0 | 0 | 0 | 0 |

Thus, the state of switch SW1 is controlled by the PBC<0> input bit value to pixel logic block 1052, the state of switch SW2 is controlled by the PBC<1> input bit value to pixel logic block 1052, the states of switches SW3, SW5, and SW6 are controlled by the PBC<2> input bit value to pixel logic block 1052, and the state of SW4 is controlled by the PBC<3> input bit value to pixel logic block 1052.

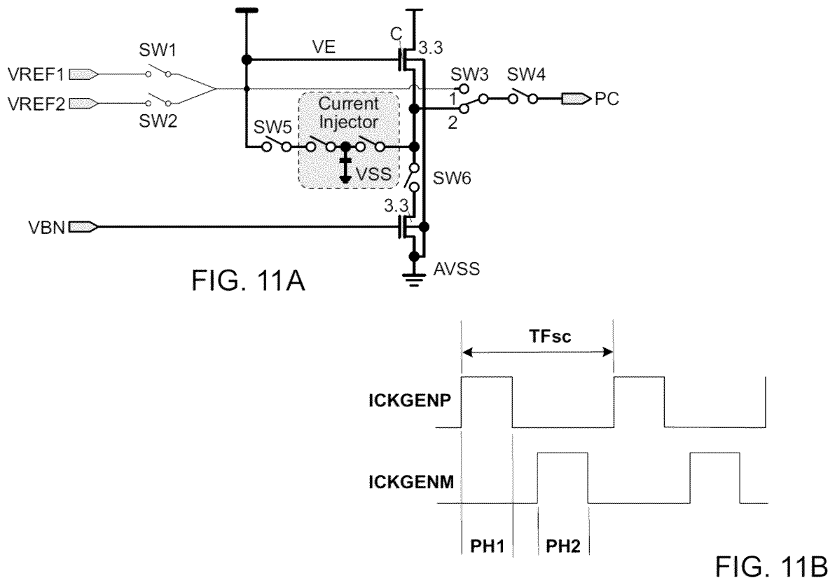

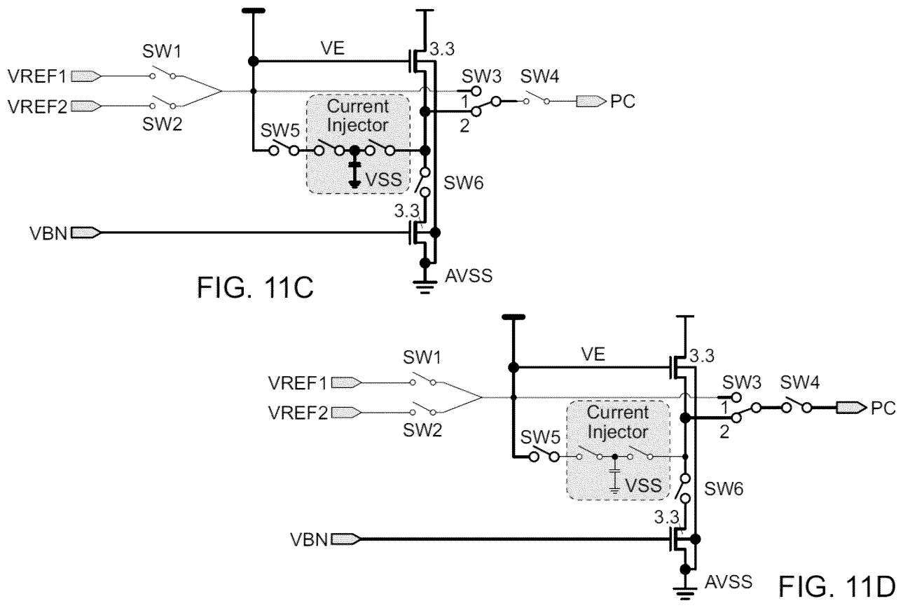

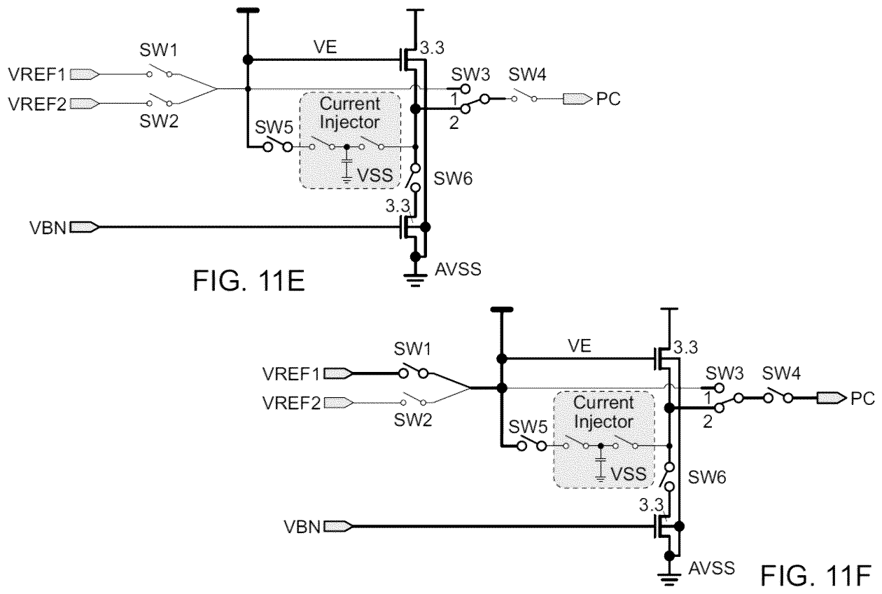

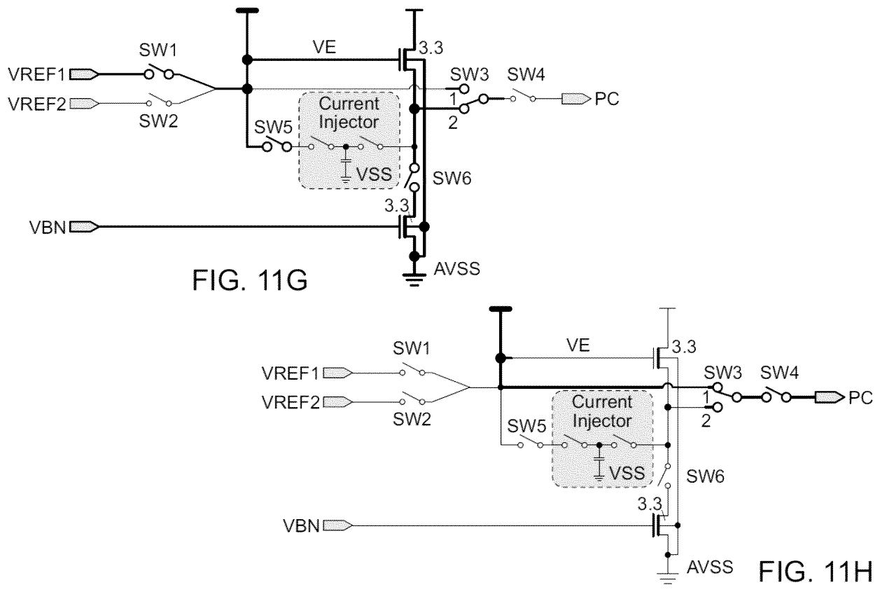

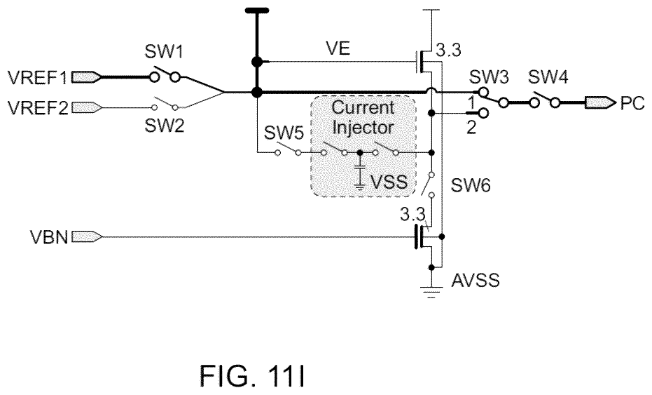

As noted previously, pixel circuit 1050 can configure electrode 902 to operate in a variety of different modes, as summarized in Table 1. In mode 1, pixel analog circuit 1054 is configured to deliver to electrode 902 a current signal biased at a voltage level V_{E}. The current signal can be used, for example, to pattern cells that are placed on top of measurement device 100. FIG. 11A is a schematic diagram showing the mode 1 configuration of pixel analog circuit 1054, with active circuit paths in heavy black lines for clarity. To enable current injection, two clock signals—ICKGENP and ICKGENM—are applied to switches 1024 and 1026 of current injector 1022, respectively. Clock signal IGENCKP is a phase 1 clock signal with positive non-overlap with clock signal IGENCKM, and clock signal IGENCKM is a phase 2 clock signal with positive non-overlap with clock signal IGENCKP. Each of the clock signals has a frequency F_{SC}. FIG. 11B is a plot showing an example of the two clock signals that are applied during mode 1.

The overall capacitance of current injector 1022 generally consists of the drain to bulk and source to bulk capacitances of the switches 1024 and 1026, and the additional metal-to-metal capacitance of capacitor 1028. Under typical operating conditions, for example, current injector 1022 generates a current signal with a magnitude of 5 nA at a frequency F_{SC} of 2 MHz.

In this mode, for diagnostic purposes, the source follower portion of the pixel analog circuit 1054 is connected to terminal 1020, so that the current injector signal can be observed for diagnostic purposes at a terminal of a peripheral circuit 908.

Switches 1024 and 1026 of current injector 1022 are typically implemented with NMOS transistors. Switch SW5 isolates electrode 902 from the current signal biased at voltage level V_{E} generated by current injector 1022 when electrode 902 is not operated in mode 1 (e.g., when current is not injected during cell patterning on measurement device 1000). In some embodiments, switch SW5 can also be implemented as a NMOS device, and may therefore establish an upper limit on the value of the V_{E} bias voltage for the current signal.

The configuration of pixel analog circuit 1054 in mode 2 is similar to the configuration for mode 1, and is shown schematically in FIG. 11C, with active circuit paths in heavy black lines for clarity. In mode 2, electrode 902 is isolated from terminal 1020 (e.g., the output terminal connected to a peripheral circuit 908) by maintaining switch SW4 in an open state. This mode is useful to prevent interactions among multiple electrodes 902 that are connected to the same peripheral circuit 908. For example, for multiple electrodes 902 that can selectively connect to a common peripheral circuit 908, one of the electrodes 902 can be configured for operation in mode 1 (i.e., active current injection), while the other electrodes 902 that can selectively connect to the common peripheral circuit 908 can be configured for operation in mode 2 to prevent interactions with the electrode 902 that is operating in mode 1.

Mode 3 corresponds to an open circuit potential (OCP) measurement mode, and is shown schematically in FIG. 11D, with active circuit paths in heavy black lines for clarity. In this mode, the electrode voltage signal, biased at a voltage magnitude V_{E}, is buffered by the NMOS source follower portion of the pixel analog circuit 1054, and electrode 902 is connected to terminal 1020, so that the voltage signal at electrode 902 can be measured by a peripheral circuit 908. For example, during operation, the bias current of the source follower portion of the pixel analog circuit 1054 is set to value that results in the source follower portion of pixel analog circuit 1054 having a relatively high output impedance (e.g., several kOhms), which allows the capacitive load (e.g., voltage signal) of the electrode to be driven to a peripheral circuit 908 connected to terminal 1020.

Mode 4 corresponds to an open circuit mode that is similar to mode 3, except that SW4 is open so that the electrode voltage signal is not routed to terminal 1020. FIG. 11E is a schematic diagram showing pixel analog circuit 1054 configured in mode 4, with active circuit paths in heavy black lines for clarity.

As discussed above in connection with modes 1 and 2, mode 4 can be used together with mode 3 when multiple electrodes 902 share a common peripheral circuit 908, to prevent interactions among the multiple electrodes 902. In particular, by maintaining electrode 902 in mode 4, the electrode voltage can be allowed to settle at terminal T2 of switch SW3, before being actively measured by a peripheral circuit 908 when switch SW4 is closed (i.e., to change the configuration of pixel analog circuit 1054 to mode 3).

As will be described subsequently, measurement signals at electrode 902 are detected by a peripheral circuit 908. However, within integrated circuit 102, there are generally fewer peripheral circuits 908 than electrodes 902. Accordingly, electrodes 902 can be selectively connected to peripheral circuits 908, such that electrodes 902 generally share peripheral circuits 908. To detect a measurement signal from a specific electrode 902, the electrode 902 is selectively connected to a peripheral circuit 908. Other electrodes 902 that share the same peripheral circuit 908 are nominally disconnected from the peripheral circuit 908 while the peripheral circuit detects the measurement signal from the specific electrode. In terms of the operating modes described above, the specific electrode 902 from which the measurement signal is detected is configured for operation in mode 3, while the other electrodes that share peripheral circuit 908 are configured for operation in mode 4.

To detect measurement signals from an array of electrodes within an integrated circuit 102, the peripheral circuits 908 of integrated circuit 102 are each selectively connected to one of the electrodes 902. For example, in an integrated circuit with P peripheral circuits 908, P electrodes 902 are initially connected to the peripheral circuits—one electrode connected to each peripheral circuit. Each of the connected electrodes 902 can be configured for operation in mode 3, and measurement signals from each of the connected electrodes can be detected by the peripheral circuits.

In general, the P electrodes 902 at which measurement signals are detected represent only a subset or “frame” of the total number of electrodes in integrated circuit 102. Measurement signals are detected from the electrodes of the integrated circuit in frame-by-frame fashion, whereby in a first frame, measurement signals are detected from a first subset of electrodes, and then in a second (and subsequent) frame(s), measurement signals are detected from additional subsets of electrodes. Each subset of electrodes corresponding to a new frame is selectively connected to the P peripheral circuits 908 which detect the measurement signals, while the other electrodes of the integrated circuit are disconnected from the peripheral circuits.

In the context of modes 3 and 4 described above, the active frame of electrodes—the subset of electrodes from which measurement signals are detected—can be configured for operation in mode 3 and selectively connected to peripheral circuits 908, which detect measurement signals from the active frame of electrodes. The other electrodes of integrated circuit 102 can be configured for operation in mode 4, which can have certain advantages. First, by configuring electrodes for operation in mode 4, the electrodes are prevented from interacting with other electrodes of the integrated circuit, and in particular, from perturbing measurement signals that are detected from electrodes in the active frame. Second, measurement signals at electrodes 902 frequently exhibit some “settling” over time; that is, they reach steady-state values gradually. By configuring electrodes in mode 4 prior to detecting measurement signals from the electrodes, the measurement signals can be allowed to settle to steady-state values before they are detected by the peripheral circuits, resulting in more accurate and reproducible measurements.

In this manner, while measurement signals from an active frame of electrodes are being detected, the subset of electrodes that form the next measurement frame within integrated circuit 102 can be prepared for measurement by configuring the electrodes of the next frame in mode 4 and allowing their measurement signals to settle to steady-state values before they are detected. After measurement signals from the current frame of electrodes have been detected by the peripheral circuits, electrodes of the current frame can be changed to operate in mode 4, and electrodes of the next measurement frame can be changed to operate in mode 3, which effectively shifts the current frame within the array of electrodes in integrated circuit 102.

In the foregoing manner, measurement signals from the electrodes of integrated circuit 102 can be detected frame-wise (i.e., groupwise) from batches or subsets of multiple electrodes, and while a particular frame of electrode measurement signals is being detected by the peripheral circuits from a group of electrodes configured for operation in mode 3, one or more subsequent groups of electrodes can be prepared for measurement signal detection by configuring the groups of electrodes for operation in mode 4.

Mode 5 corresponds to a buffer calibration mode for the voltage signal V_{REF1} that is received by pixel analog circuit 1054 at terminal 1016. FIG. 11F is a schematic diagram of this mode, with active circuit paths in heavy black lines for clarity. This mode can be used for calibration purposes. Reference voltage V_{REF1} is applied to electrode 902, and the output is directed to terminal 1020, where it can be detected by a peripheral circuit 908. Measurement of the output signal from electrode 902 can be used to correct, for each electrode 902, the source follower offset (e.g., bias) from the actual open circuit potential value, so that corrections to measured open circuit potential signals from electrodes 902 can be individually corrected.

Mode 6, which is illustrated schematically in FIG. 11G with active circuit paths in heavy black lines for clarity, is similar to mode 5 except that the output from electrode 902 is disconnected from terminal 1020. In this manner, the relationship between modes 5 and 6 is analogous to the relationship between modes 1 and 2, and to the relationship between modes 3 and 4. During calibration, electrodes that are being actively calibrated can be configured for operation in mode 5 and connected to a peripheral circuit 908. To prevent interaction between electrodes during calibration, electrodes that are not actively being calibrated can be configured for operation in mode 6.

Mode 7 corresponds to a buffer calibration mode for the voltage signal V_{REF2} that is received by pixel analog circuit 1054 at terminal 1018. The configuration of pixel analog circuit 1054 in mode 7 is similar to mode 5, except that V_{REF2} is applied to electrode 902, with the output signal from electrode 902 directed to terminal 1020.

Mode 8 is similar to mode 7, except that the output from electrode 902 is disconnected from terminal 1020. The relationship between modes 7 and 8 is analogous to the relationship between modes 5 and 6 described above, except that reference voltage signal V_{REF2} is directed to electrode 902. Similar considerations apply to mode selection during calibration of electrodes 902 to correct for the source follower offset from the actual open circuit potential value.

Mode 9 is a current measurement mode. FIG. 11H is a schematic diagram showing pixel analog circuit 1054 configured in mode 9, with active circuit paths in heavy black lines for clarity. In this mode, the measurement signal at electrode 902 is directed to terminal 1020, where it is coupled into a peripheral circuit 908. To measure the electrical current at electrode 902 biased at a known potential, peripheral circuit 908 functions as a transimpedance amplifier, amplifying the current measurement signal from electrode 902.

Mode 10 is a voltage supply mode, in which electrode 902 is directly biased with voltage signal V_{REF1} received at terminal 1016 of pixel analog circuit 1054. FIG. 11I is a schematic diagram showing pixel analog circuit 1054 configured in mode 10, with active circuit paths in heavy black lines for clarity. In this mode, V_{REF1} is delivered directly to electrode 902. Measurement signals from another electrode (or group of electrodes) are detected by one or more peripheral circuits 908 by routing the measurements signals from that/those electrode(s) to the peripheral circuit(s) 908.

Mode 11 is similar to mode 10, except that switch SW4 is open so that the output signal from electrode 902 is not routed to a peripheral circuit. Mode 11 is used to prevent interaction with electrodes in mode 10 in a manner similar to the relationship between modes 1 and 2, modes 3 and 4, and modes 5 and 6 discussed above.

Mode 12 is a voltage supply mode in which electrode 902 is directly biased with voltage signal V_{REF2} received at terminal 1018 of pixel analog circuit 1054. Otherwise, mode 12 is similar to mode 10. Measurement signals from another electrode (or group of electrodes) are detected by one or more peripheral circuits 908 by routing the measurements signals from that/those electrode(s) to the peripheral circuit(s) 908.

Mode 13 is similar to mode 12, except that switch SW4 is open so that the output signal from electrode 902 is not routed to a peripheral circuit. Mode 13 is used to prevent interaction with electrodes in mode 12 in a manner similar to the relationship between modes 1 and 2, modes 3 and 4, and modes 5 and 6 discussed above.

Combinations of modes 9, 11, and 13 can be used to measure “cross-electrode impedance” among groups of electrodes in integrated circuit 102. For example, in certain measurements, one or more of electrodes 902 are configured for operation in mode 9, and function as measurement electrodes at which electrical current is measured as described above. Additionally, one or more of electrodes 902 are configured for operation in mode 11 or mode 13, and function as shielding electrodes to control the electric field distribution at the surface of measurement device 100. Further still, one or more of electrodes 902 are configured for operation in mode 13 or mode 11 and function as return electrodes for the electric field.

The choice of spatial arrangement for the groups of electrodes that are configured in different modes as described above depends on a number of factors including, but not limited to, the nature of the measurements that are made and the associated biological attributes of a sample that are interrogated by the measurements. Specific examples of different arrangements of electrodes operating in different modes for performing different measurements are described in more detail below.

Mode 14 is an isolated electrode condition in which electrode 902 is electrically isolated from the reference voltages supplied at terminals 1016 and 1018, from the current injector 1022, and from peripheral circuits 908. This mode can be used to isolate electrode 902 from other electrodes in integrated circuit 102 in a manner analogous to other modes such as 2, 4, and 6. For example, during detection of current measurement signals at one or more other electrodes, electrode 902 can be configured for operation in mode 14 to ensure that electrode 902 does not interact with the one or more other electrodes at which measurement signals are being detected.

### (b) Electrode Measurement Geometries

As discussed above, each of electrodes 902 is connected to a dedicated pixel circuit 1050, and therefore each of the electrodes can be configured for operation in any of the different operating modes described. Moreover, it should be understood that the operating modes shown in Table 1 are merely examples, and it is also possible to configure each of electrodes 902 in additional operating modes, depending upon the nature of specific measurements that are performed. The configuration of each electrode is established by the four bit values PBC<3:0>, which are used by pixel logic block 1052 to control the states of switches SW1-SW6 of pixel analog circuit 1054.

Multiple operating modes in Table 1 can be used to detect measurement signals at electrode 902. In other words, electrode 902 can be configured for operation in one of multiple different operating modes, and once configured, a peripheral circuit 908 to which electrode 902 is connected through terminal 1020 can detect a measurement signal at electrode 902.

Because integrated circuit 102 contains fewer peripheral circuits 908 than electrodes 902, measurement signals are detected from electrodes 902 in groups or frames by the peripheral circuits 908, as described above. The number of electrode measurement signals that can be detected in parallel depends on the number of peripheral circuits 908 present in integrated circuit 102, as each peripheral circuit 908 is selectively connected to only one electrode 902 at any time, and can therefore detect a measurement signal only from the electrode to which it is connected.

In some embodiments, measurement signals are detected from groups of adjacent electrodes in integrated circuit 102, such that the effective spatial resolution of the measurement signals corresponds to the spacing between adjacent electrodes (i.e., Δx and Δy) in the electrode array. By detecting measurement signals from groups or frames of electrodes 902 in parallel, the overall measurement time for detection of signals across the entire electrode array can be reduced, relative to the time required to sequentially detect measurement signals at each electrode in the array.

In certain embodiments, measurement signals can also be detected from groups of electrodes that do not constitute a contiguous block of adjacent electrodes in the electrode array of integrated circuit 102. By detecting measurement signals in parallel from a group of electrodes 902 that are not all nearest neighbors within the electrode array, the effective spatial resolution of the detected measurement signals can be adjusted. Effectively, the electrode pitch can be adjusted to a larger (i.e., coarser) value. Measurements performed in this manner can be useful for a variety of applications. For example, to facilitate certain measurements, an initial scan of the electrode array of integrated circuit 102 can be performed at coarser (i.e., lower) spatial resolution to sample measurement signals across the electrode array more rapidly than would otherwise be possible at the finest spatial measurement resolution.

To further illustrate the process by which the effective spatial measurement resolution can be adjusted, consider an electrode array of an integrated circuit 102 in which electrodes 902 are arranged in a regular, square array pattern, with a 12.5 micrometer spacing between adjacent electrodes in both orthogonal array directions (i.e., Δx=12.5 micrometers, Δy=12.5 micrometers). It should be noted that these electrode spacings are merely illustrative; more generally, a wide variety of different electrode spacings can be used, as described above. Further, in this example, integrated circuit 102 contains 9 peripheral circuits 908 that are used to detect measurement signals from electrodes 902 in the array. As will be explained in more detail below, in general, the number of peripheral circuits 908 in integrated circuit 102 can vary within a wide range according to various performance, fabrication, and power consumption criteria.

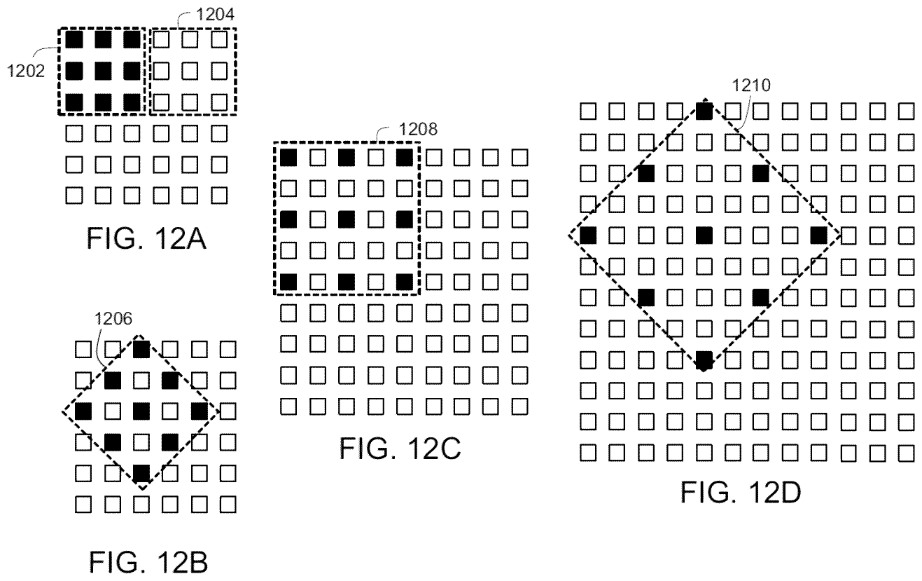

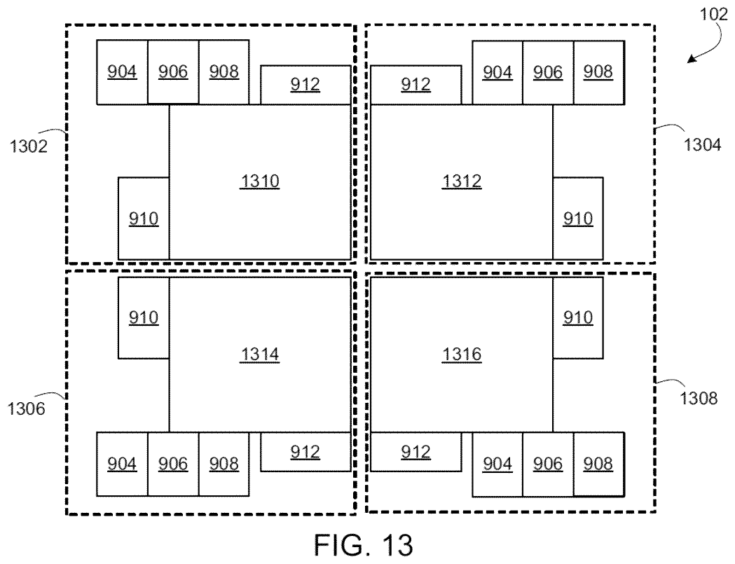

FIGS. 12A-12D are schematic diagrams that show a portion of the electrode array in this example. In each of these figures, measurement signals are detected from different electrodes of the array in a manner such that the effective spatial resolution of the measurement signals varies among the figures. In FIG. 12A, measurement signals from groups of immediately adjacent electrodes are detected. Referring to the figure, in a first measurement frame, each of the electrodes in region 1202 (9 electrodes in total) is selectively connected to a different one of the 9 peripheral circuit 908, and measurement signals from each of the electrodes of region 1202 are detected by the peripheral circuits. Then, in a second measurement frame, each of the electrodes in region 1204 (9 electrodes in total) is selectively connected to a different one of the 9 peripheral circuits 908, and measurement signals from each of the electrodes of region 1204 are detected by the peripheral circuits. This process continues until measurement signals from each of the electrodes in the array have been measured. Because signals detected from immediately adjacent electrodes, the effective measurement resolution in both array directions is 12.5 micrometers, i.e., the maximum array resolution corresponding to the minimum spacing between detected measurement signals in the array.

FIG. 12B illustrates a detection method in which measurement signals are detected at lower resolution. Measurement signals are detected from electrodes in each row and column of the electrode array. However, within each row and column, measurement signals are detected only from every other (e.g., every second) electrode. That is, within each row and each column of the electrode array, measurement signals are not detected from any pair of electrodes that are immediately adjacent. Thus, referring to FIG. 12B, in a first measurement frame 1206, the 9 shaded electrodes are each selectively connected to a different one of the 9 peripheral circuits 908, and measurement signals from the 9 connected electrodes are detected by the peripheral circuits. The effective spatial resolution of the detected measurement signals is the diagonal nearest neighbor distance, sqrt(2)*12.5 microns=17.7 microns. Following the detection of measurement signals from each of the 9 shaded electrodes shown in FIG. 12B, a second measurement frame consisting of 9 different electrodes, with relative positions analogous to those of the 9 shaded electrodes in FIG. 12B, is selected. The electrodes of the second measurement frame are each selectively connected to one of the 9 peripheral circuits 908, and measurement signals corresponding to the second measurement frame are detected. This procedure is repeated across the electrode array, yielding measurement signals at lower effective spatial resolution than in FIG. 12A.

FIG. 12C illustrates a detection method in which measurement signals are detected at even lower spatial resolution. Within the electrode array, measurement signals are detected only from electrodes in alternating rows and columns within each measurement frame. Further, in each row and column of the measurement frame in which measurement signals are detected from electrodes, measurement signals are only detected from every other (e.g., every second) electrode. An example measurement frame 1208 is illustrated in FIG. 12C. In this method, measurement signals are only detected from electrodes that are next nearest neighbors along both the array row and column directions and the array diagonal directions.

In measurement frame 1208, the 9 shaded electrodes are each selectively connected to a different one of the 9 peripheral circuits 908, and measurement signals from the 9 connected electrodes are detected by the peripheral circuits. The effective spatial resolution of the detected measurement signals is the next nearest neighbor distance along the array row and column directions, 2*12.5 microns=25.0 microns. Following the detection of measurement signals from each of the 9 shaded electrodes shown in FIG. 12C, a second measurement frame consisting of 9 different electrodes, with relative positions analogous to those of the 9 shaded electrodes in FIG. 12C, is selected. The electrodes of the second measurement frame are each selectively connected to one of the 9 peripheral circuits 908, and measurement signals corresponding to the second measurement frame are detected. This procedure is repeated across the electrode array, yielding measurement signals at an effective spatial resolution even lower than in FIG. 12B.

FIG. 12D illustrates a detection method in which measurement signals are detected at still lower spatial resolution. In the method shown in FIG. 12D, measurement signals are detected only from electrodes in every other (e.g., every second) row and column of the electrode array. Further, within each row and column of the measurement frame in which measurement signals are detected from electrodes, measurement signals are only detected from every fourth electrode. In the example measurement frame 1210 of FIG. 12D, measurement signals are detected only from the shaded electrodes. As such, the effective spatial resolution of the detected signals is the next nearest diagonal neighbor distance within the electrode array.

In measurement frame 1210, the 9 shaded electrodes are each selectively connected to a different one of the 9 peripheral circuits 908, and measurement signals from the 9 connected electrodes are detected by the peripheral circuits. The effective spatial resolution of the detected measurement signals is 2*sgrt(2)*12.5 microns=35.4 microns. Following the detection of measurement signals from each of the 9 shaded electrodes shown in FIG. 12D, a second measurement frame consisting of 9 different electrodes, with relative positions analogous to those of the 9 shaded electrodes in FIG. 12D, is selected. The electrodes of the second measurement frame are each selectively connected to one of the 9 peripheral circuits 908, and measurement signals corresponding to the second measurement frame are detected. This procedure is repeated across the electrode array.

As will be appreciated, the pattern of electrode selection for detection of measurement signals can be extended to still larger electrode spacings within the array, such that the effective spatial resolution of the detected measurement signals can be varied widely. Obtaining measurement signals at relatively lower spatial resolution can be an effective method, for example, to perform an initial interrogation or sampling of measurement signals across the electrode array. The initial interrogation or sampling can reveal, for example, which portions of the array generate measurement signals of interest, and which portions of the array do not. Further measurement signals can then be induced and detected from the portions of the array that generate measurement signals of interest (for example, measurement signals can be induced by applying different electrical stimuli to the electrodes in those portions of the array, and/or measurement signals can be detected at higher effective spatial resolution from electrodes in those portions of the array as discussed above).

### (c) Integrated Circuit Functional Groups

In some embodiments, to facilitate fabrication of integrated circuit 102, the integrated circuit can be implemented as a multiplicity of repeating functional groups. Dividing integrated circuit 102 in such a manner facilitates fabrication, as the integrated circuit can be formed on a substrate simply by replicating a functional group f times, where f represents the number of repeating functional groups in the integrated circuit.

FIG. 13 is a schematic diagram showing a representative example of an integrated circuit 102 that includes a plurality of functional groups. In the example of FIG. 13, integrated circuit 102 includes f=4 functional groups, each of which is referred to as a “quad.” It should be understood that the integrated circuit shown in FIG. 13—and more particularly, the number of functional groups—is merely an example. In general, the number of functional groups f within integrated circuit can be selected as desired to facilitate fabrication and balance performance factors such as detection speed and power consumption. Thus, f can generally be 1 or more (e.g., 2 or more, 4 or more, 8 or more, 16 or more, 32 or more, 64 or more, 128 or more, or even more).

As used herein, a “functional group” that is part of integrated circuit is a collection of components that includes one or more electrodes, one or more peripheral circuits, one or more reference amplifiers, one or more analog-to-digital converters (ADCs), an optional row select logic unit, and an optional column select logic unit. A functional group can also include other components, including any of the other components described herein. In general, each functional group is capable of operating as a self-contained integrated circuit, detecting measurement signals from electrodes within the functional group.

As noted above, integrated circuit 102 in FIG. 13 includes four functional groups 1302, 1304, 1306, and 1308. Each functional group includes one or more ADCs 904, one or more REAs 906, one or more PCs 908, an optional row select logic unit 910, and an optional column select logic unit 912. In addition, each functional group includes a portion of the electrode array of integrated circuit 102. Functional group 1302 includes a first portion 1310 of the electrode array, functional group 1304 includes a second portion 1312 of the electrode array, functional group 1306 includes a third portion 1314 of the electrode array, and functional group 1308 includes a fourth portion 1316 of the electrode array. In some embodiments, as shown in FIG. 13, the portions of the electrode array are positioned within the functional groups such the portions are adjacent to each other, forming a contiguous electrode array within integrated circuit 102.

In general, each functional group can include one or more (e.g., two or more, three or more, four or more, five or more, six or more, seven or more, eight or more, nine or more, 10 or more, 12 or more, 15 or more, 20 or more, 25 or more, 30 or more, 50 or more, or even more) ADCs 904. Each ADC connects to one or more PCs 908 and/or one or more REAs 906 in the functional group, and functions to convert analog received signals into digital signals for transmission to an external device such as host controller 706. For example, measurement signals from one or more electrodes of the portion of the electrode array of the functional group can be detected by PCs 908, and then routed to ADCs 904 for conversion into digital signals, which are then communicated to host controller 706.

The input of each ADC is connected to a multiplexer, which is in turn connected to one or more peripheral circuits 908 and/or one or more reference electrode amplifiers 906. As described above, the one or more peripheral circuits 908 selectively connect to electrodes 902 of the portion of the electrode array of the functional group.

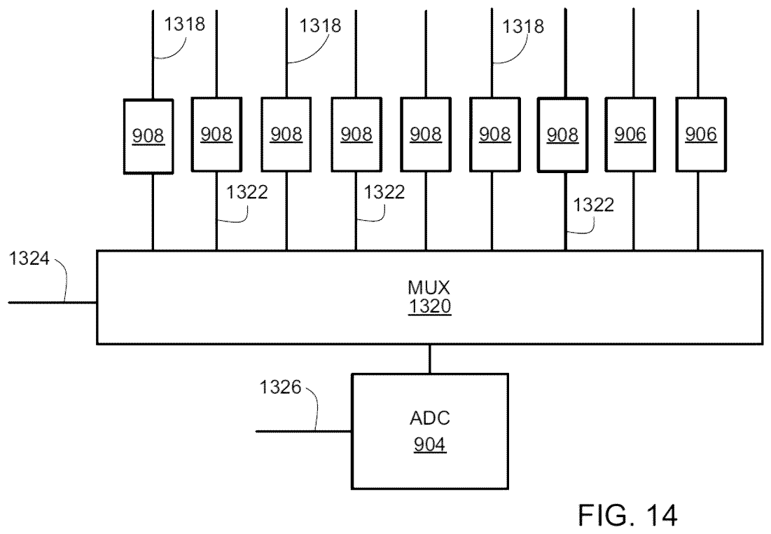

FIG. 14 is a schematic diagram showing an example portion of a functional group that includes a multiplexer 1320 connected to ADC 904. Multiplexer 1320 is also connected, via conductive lines or traces 1322, to PCs 908 and REAs 906 within the functional group. In turn, PCs 908 are selectively connected via conductive lines or traces 1318 to electrodes of the functional group. It should be noted that the number of conductive lines, PCs, and REAs in FIG. 14 is merely an example provided for illustrative purposes.

Multiplexer 1320 functions as a signal router, directing signals from PCs 908 and/or REAs 906 into ADC 904. After conversion from analog signals into digital signals in ADC 904, the digital signals are routed via data output line 1326 to a data output interface, as will be described in greater detail subsequently. Multiplexer 1320 receives control signals via conductive line or trace 1324 (e.g., from host controller 706), and responsive to the control signals, selectively directs detected measurement signals from PCs 908 and/or signals from REAs 906 to ADC 904.

By selectively connecting different groups of electrodes to the PCs of the functional group, the same PCs can be used to detect—in sequential groups—measurement signals from all of the electrodes of the portion of the electrode array within the functional group. In effect, the PCs of the functional group are shared for detection purposes among the electrodes of the functional group. In this manner, measurement signals from a relatively large number of electrodes can be detected by a comparatively small number of PCs. Further, once detected, the measurement signals can be converted into digital form and packed into a sequential data stream for transmission to an external device such as host controller 706, as described in more detail below.

Each functional group can generally include one or more (e.g., two or more, three or more, four or more, five or more, six or more, seven or more, eight or more, nine or more, 10 or more, 12 or more, 15 or more, 20 or more, 25 or more, 30 or more, 50 or more, or even more) REAs 906.

Each REA 906 can selectively connect to any of the electrodes 902 in the portion of the electrode array within the functional group. REAs 906 can each deliver stimulation signals to electrodes 902 and/or detect measurement signals at electrodes 902, and more generally, perform functions similar to PCs 908. In some embodiments, for example, the structure of REA 906 is similar to, or even the same as, PC 908. Examples of peripheral circuit structures are described in greater detail below.

Each functional group includes one or more (e.g., two or more, three or more, four or more, five or more, six or more, seven or more, eight or more, nine or more, 10 or more, 12 or more, 15 or more, 20 or more, 25 or more, 30 or more, 50 or more, or even more) PCs 908. PCs perform a variety of functions, including detection of measurement signals from electrodes and calibration of the integrated circuit. The various operating modes of PCs 908 will be discussed in more detail below.

In general, as shown in FIG. 14, conductive lines 1322 function as input lines to the PCs. With reference to the electrodes of a functional group that form a portion of the electrode array of integrated circuit 102, each PC is selectively connectable through ADC 904 and multiplexer 1320 to each of multiple sets of electrodes in the portion of the electrode array. The sets of electrodes to which a particular PC is selectively connectable can, in some embodiments, follow a repeating pattern. Thus, in some embodiments for example, where a functional group includes n PCs 908, each of the PCs is selectively connectable to every n-th set of electrodes.

The connection between a particular PC 908 and a set of electrodes is shared (i.e., electrically shorted) between all electrodes in the set. Each of these shared connections is cross-connected to another subset of the electrodes in the functional group. During detection of measurement signals, the cross-connections allow each PC 908 to be selectively connected to one or more electrodes for detection of measurement signals at the one or more electrodes.

In some embodiments, a PC is selectively connected to, and detects a measurement signal from, only one electrode at a time. For example, in a functional group with n PCs, in a first measurement frame each PC is selectively connected to, and detects a measurement signal from, a different one of a first group of n electrodes. In a second measurement frame, each PC is selectively connected to, and detects a measurement signal from, a different one of a second group of n electrodes, where the electrodes of the second group are different from the electrodes of the first group. In each successive measurement frame, a different group of n electrodes are selectively connected to the PCs and measurement signals from the electrodes are detected, until measurement signals have been detected from all of the electrodes in the functional group.

In certain embodiments, a PC is selectively connected to more than one electrode at the same time for detection of measurement signals. For example, certain types of measurement signals can be detected additively in parallel at multiple electrodes. A PC is connected at the same time to each of the multiple electrodes, and measurement signals from the multiple electrodes are routed in parallel into the PC. The detected signal is additive with respect to the incoming measurement signals. This type of configuration can be used, for example, to make location-averaged measurements of a sample in response to electrical stimuli.

As discussed above, each electrode 902 is connected to a peripheral circuit 908 through a pixel circuit 1054. Specifically, terminal 1020 of the pixel circuit is connected to terminal 1506 of peripheral circuit 908. In general, the pixel circuits associated with multiple electrodes 902 are connected in parallel to the common terminal 1506 of a single PC 908.

Consider, for example, a group of p electrodes Eᵢ (where i=1, 2, 3, . . . p), each of which is connected through its own pixel circuit to the same PC 908 through the PC's terminal 1506. To direct a measurement signal from only one of the electrodes, E₁ for example, to PC 908, the pixel circuit connected to electrode E₁ is operated in a mode in which the measurement signal from electrode E₁ is routed to the output terminal 1020 of the pixel circuit. The pixel circuits connected to electrodes E₂, E₃, . . . Eₚ are operated in modes in which measurement signals from electrodes E₂, E₃, . . . Eₚ are not routed to their output terminals 1020. Consequently, only the measurement signal from electrode E₁ is received at terminal 1506 of PC 908. After the measurement signal has been routed by PC 908 to ADC 904, the operating modes of the pixel circuits connected to the electrodes can be changed. For example, the operating mode of the pixel circuit connected to electrode E₁ can be changed to a mode in which the measurement signal from electrode E₁ is not routed to the output terminal 1020 of the pixel circuit, and the operating mode of the pixel circuit connected to electrode E₂ can be change to a mode in which the measurement signal from electrode E₂ is routed to the output terminal 1020 of the pixel circuit. PC 908 can then be used to detect the measurement signal from electrode E₂, routing it to ADC 904.

To direct measurement signals from multiple electrodes to PC 908, the pixel circuits connected to multiple electrodes—for example, electrodes E₁, E₂, E₃, and E₄—can be configured to operate in modes in which the measurement signals from each of these electrodes are routed to the output terminals 1020 of the corresponding connected pixel circuits. At terminal 1506, PC 908 therefore receives the measurement signals from electrodes E₁, E₂, E₃, and E₄ in parallel. The pixel circuits connected to electrodes E₅, E₆, . . . Eₚ are configured to operate in modes in which measurement signals from these electrodes are not routed to the output terminals 1020 of the corresponding connected pixel circuits. Thus, at terminal 1506, PC 908 does not receive measurement signals from these electrodes.

After PC 908 detects signals from electrodes E₁, E₂, E₃, and E₄ and routs the measurement signals to ADC 904, the configurations of the pixel circuits can be changed in the manner described above and measurement signals from a different set of electrodes from among E₁-Eₚ can be selectively routed to terminal 1506 of PC 908.

As discussed above, the number of ADCs, REAs, and PCs in each functional group can vary. The choice of how many of these components are present accounts for factors such as complexity of integrated circuit 102, power consumption of the circuit, desired detection speed for measurement signals, and other factors. In general, by increasing the number of ADCs, REAs, and PCs in a functional group, measurement signals from electrodes of the functional group can be detected more rapidly, because more of the signals can be detected in parallel rather than sequentially. However, increasing the numbers of these components also significantly increases circuit complexity and power consumption.

The number of PCs 908 connected to each ADC 904 can also be controlled. As shown in FIG. 14, multiplexer 1320 can receive control signals via conductive line 1324, and responsive to such control signals, can selectively direct signals from each of the PCs 908 and each of the REAs 906 to ADC 904.

For detection of measurement signals from electrodes at greatest speed, measurement signals can be detected by each of the PCs 908 and routed through ADC 904. However, to conserve power, or for measurements that can be performed at prolonged intervals with relatively low sampling rates and/or at coarser spatial resolution, some of PCs 908 can be selectively disabled, and measurement signals can be detected by only a subset of PCs 908 and routed through ADC 904.

As described above, each PC 908 can be selectively connected to multiple electrodes. During a particular measurement frame in which measurement signals are detected by PCs 908, each PC 908 detects measurement signals from one or more electrodes connected to the PC, while at the same time, one or more electrodes connected to the PC are operated in a mode in which measurement signals are not routed to the PC. These inactive connections represent significant parasitic loads on the active connection lines between the electrodes and the PC, which results in increased settling time, feedthrough, and current leakage on the electrode(s) that is/are routing measurement signals to the PC. In some embodiments, to reduce these parasitic loads, isolation switches are connected in series with each of the connection lines between the pixel circuits of the connected electrodes and the PC 908 to which they are connected. When one or more of the electrodes is/are configured to rout measurement signals to the PC 908 via one or more of the connection lines, the other connection lines (which connect the pixel circuits of the non-routing electrodes to the PC 908) can be disconnected from the PC 908 by opening the corresponding isolation switches. To selectively adjust the isolation switches, the isolation switches can be implemented as a switch block with a dedicated control logic unit that receives control signals, e.g., from host controller 706, and opens or closes isolation switches within the switch block according to the control signals.

It should be noted that where integrated circuit 102 includes multiple functional groups, the number of ADCs, REAs, and PCs within each functional group can be independently selected. In some embodiments, to facilitate straightforward fabrication of integrated circuit 102, each functional group will have the same number of ADCs, REAs, and PCs. However, more generally, in certain embodiments one or more of the number of ADCs, the number of REAs, and the number of PCs can vary among the functional groups of the integrated circuit. Each functional group can be independently configured with ADCs, REAs, and PCs as described above.

### (d) Peripheral Circuits

Integrated circuit 102 generally includes multiple peripheral circuits 908. Peripheral circuits 908 can each be shared across a portion of or all of the integrated circuit 102, or specific to a particular functional group of the integrated circuit 102. Peripheral circuits 908 typically include operational amplifier circuits that can be reconfigured, via switches, to allow the circuits to operate in a variety of different modes.

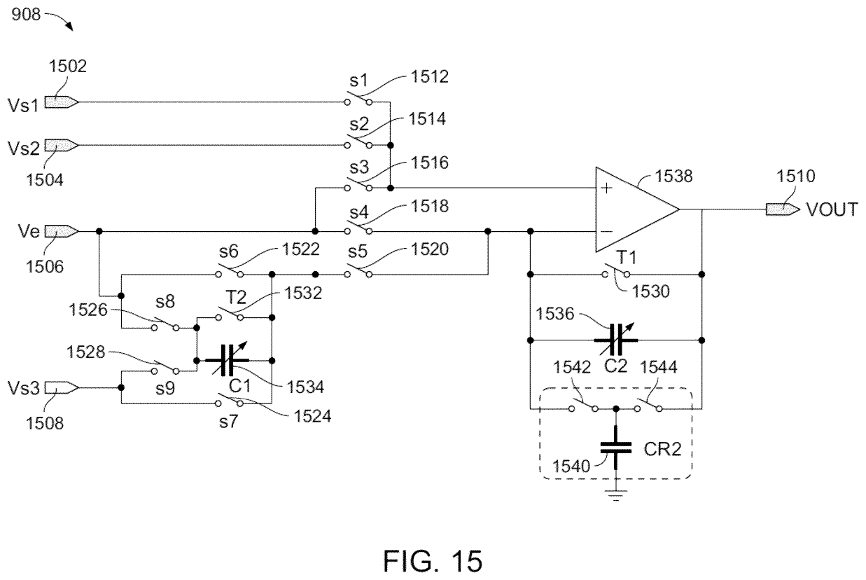

FIG. 15 is a schematic diagram of an example of a peripheral circuit 908 that can be implemented in integrated circuit 102. Peripheral circuit 908 includes terminals 1502, 1504, and 1508 which are connected to a DAC. Reference voltage signals Vₛ₁, Vₛ₂, and Vₛ₃ are received as input signals at terminals 1502, 1504, and 1508, respectively, after generation in the DAC.

Terminal 1506 is the input terminal for peripheral circuit 908, and is selectively connected to output terminal 1020 of the pixel circuit 1050 via conductive line or trace 1318. As discussed above, output terminal 1020 of the pixel circuit is directly or indirectly connected to (or connectable to) electrode 902. Measurement signals (and/or other signals) from electrode 902 are thereby routed through pixel circuit 1050 to terminal 1506 of peripheral circuit 908 as signal Vₑ.

Peripheral circuit 908 includes a plurality of switches 1512 (s1), 1514 (s2), 1516 (s3) 1518 (s4), 1520 (s5), 1522 (s6), 1524 (s7), 1526 (s8), 1528 (s9), 1530 (T1), and 1532 (T2), two adjustable capacitances 1534 (C1) and 1536 (C2), and an operational amplifier 1538, the output of which is connected to output terminal 1510. Peripheral circuit further includes a capacitor resistor CR2 (1540) that can be selectively coupled to the circuit by switches 1542 and 1544. Output terminal 1510 is connected via conductive line or trace 1322 to multiplexer 1320, as shown in FIG. 14.

Returning to FIG. 15, the various switches of peripheral circuit 908 can be used to control the operating mode of peripheral circuit 908. To enable a particular mode, a sequence of control bits representing state values (e.g., 1=closed, 0=open) for each of the switches in peripheral circuit 908 is received (e.g., from host controller 706), and a peripheral circuit logic block (not shown in FIG. 15) configures the switches of peripheral circuit 908 according to the control bit sequence. In this manner, the operating mode of each peripheral circuit 908 in the integrated circuit can be independently controlled.

In a first configuration, peripheral circuit 908 can operate in a current measurement mode, detecting a current from an electrode biased at voltage Vₛ₁. The current signal Vₑ is received at input terminal 1506. Switches s1 and s4 are closed, which switches s2, s3, and s5-s9 in peripheral circuit 908 are open.

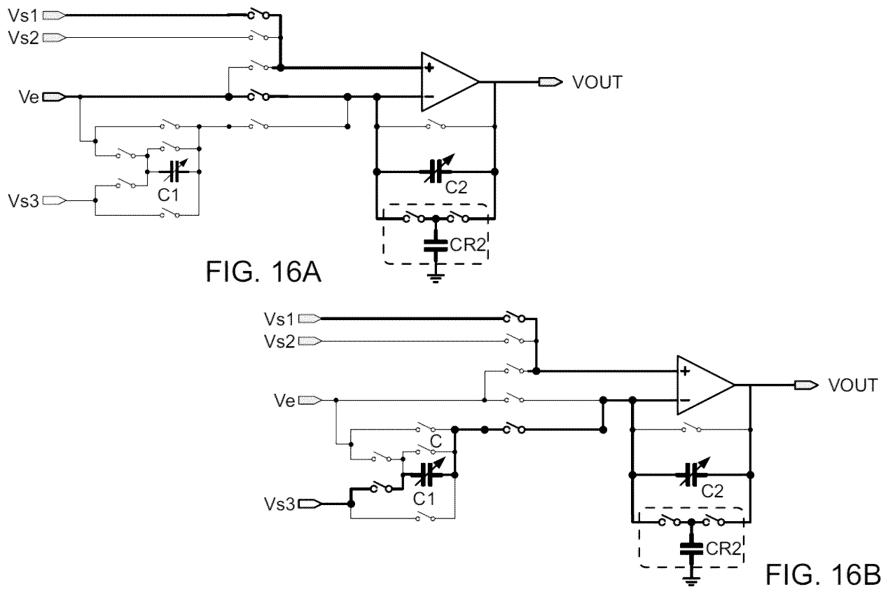

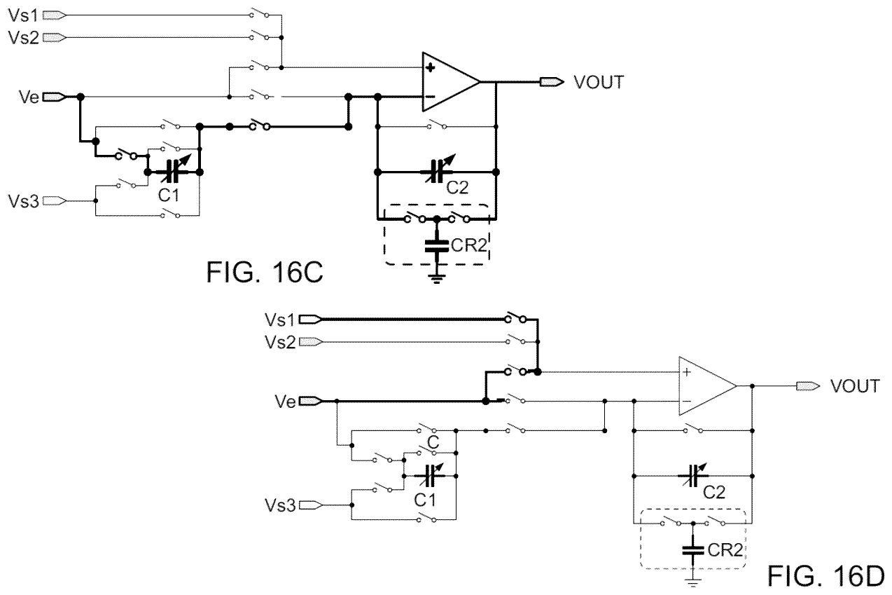

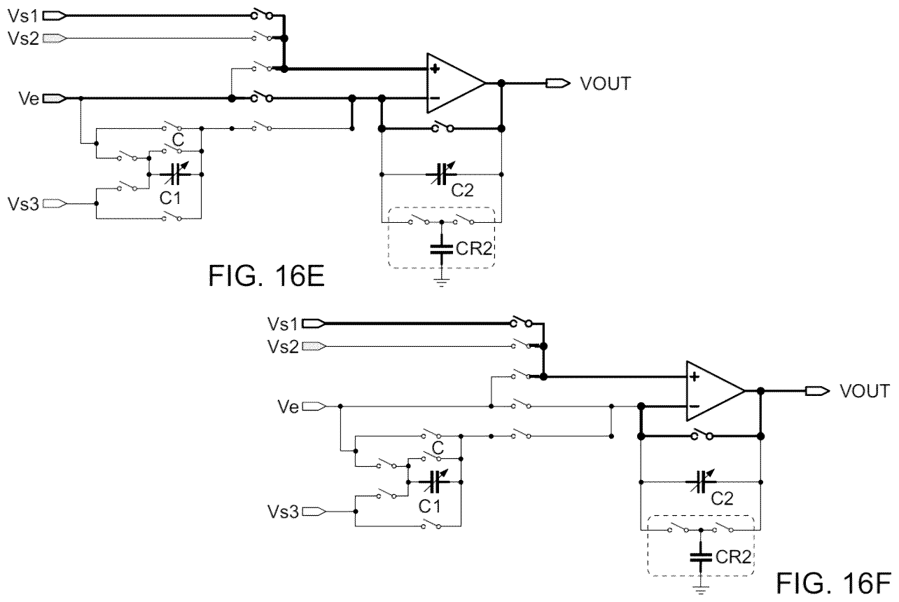

In this configuration of peripheral circuit 908, switches 1542 and 1544 are controlled with non-overlapping clock signals of frequency F. Together, switches 1542 and 1544 along with capacitor 1540 (capacitance C) function as a switched-capacitor resistance with a resistance value R=1/(C*F). FIG. 16A is a schematic diagram of peripheral circuit 908 operating in this mode, with active circuit paths shown in heavy black lines.

With switches 1542 and 1544 controlled in this manner, the switched-capacitor resistance is introduced on the feedback of operational amplifier 1538, which acts as a trans-impedance amplifier (TIA) to amplify the current signal received at terminal 1506. The transconductance gain of the TIA is controlled by the resistance value R of the switched-capacitor resistor.

Adjustable capacitance C2 is adjusted to set a feedback pole for the TIA. The input current signal at terminal 1506, either a DC or AC current signal, is converted to an output voltage signal by the TIA. The voltage signal is output at terminal 1510 as a detected measurement signal.

In a second configuration, peripheral circuit 908 can operate in a current measurement calibration mode. In this mode, the peripheral circuit is calibrated for current measurements against a known calibration signal. Switches s1, s5, and s9 are closed, switches 1542, and 1544 are driven with non-overlapping clock signals to adjust the transconductance gain of the TIA, and the other switches of PC 908 are open. FIG. 16B is a schematic diagram of peripheral circuit 908 operating in this mode, with active circuit paths shown in heavy black lines.

With switch s1 closed, the TIA amplifies an incoming current signal biased at Vₛ₁. A calibration voltage signal Vₛ₃ is received at terminal 1508, and with switch s9 closed, the calibration voltage signal is converted into a calibration current signal by passing through adjustable capacitance C1. The calibration current signal is transmitted to the TIA and detected as an output signal at terminal 1510. The detected calibration current signal can be used to verify the accuracy of detection of measured current signals by the peripheral circuit. In particular, in this mode, the measurement of both the magnitude and phase of current signals—including at different input signal frequencies—can be calibrated, allowing for accurate detection of signals from multi-frequency impedance measurements.

In a third configuration, peripheral circuit 908 can operate in an AC voltage measurement mode. Switches s5 and s8 are closed, switches s1-s4, s6-s7, and s9 are closed, and switches 1542, and 1544 are driven with non-overlapping clock signals to adjust the transconductance gain of the TIA. FIG. 16C is a schematic diagram of peripheral circuit 908 operating in this mode, with active circuit paths shown in heavy black lines.

The switched-capacitor resistor in FIG. 16C sets the high-pass filtering pole of amplifier 1538. Amplifier 1538 functions as a capacitive gain voltage amplifier with a negative gain of—C1/C2. An AC voltage signal Vₑ from an electrode is received at terminal 1506 and amplified through the C1 and C2 capacitors. The amplified voltage signal is transmitted to output terminal 1510 by the amplifier as a detected voltage signal from the electrode.

In a fourth configuration, peripheral circuit 908 can operate in a direct voltage drive mode. In this mode, the peripheral circuit is configured to directly drive an electrode with a reference voltage signal. This mode can be implemented in a variety of different ways. To drive an electrode connected through its corresponding pixel circuit to terminal 1506, switches s1 and s3 are closed, and the other switches are open. FIG. 16D is a schematic diagram of peripheral circuit 908 operating in this manner, with active circuit paths shown in heavy black lines. Reference voltage Vₛ₁, received from the DAC at terminal 1502, is directly coupled to terminal 1506, and therefore to the electrode connected to terminal 1506.

Alternatively, to drive the electrode with reference voltage Vₛ₂, switches s2 and s3 are closed and the other switches of peripheral circuit 908 are open. Reference voltage Vₛ₂, received from the DAC at terminal 1504, is thus directly coupled to terminal 1506 and the electrode.

As a further alternative, to drive the electrode with reference voltage Vₛ₃, switches s8 and s9 are closed and the other switches are open. Reference voltage Vₛ₃, received from the DAC at terminal 1508, is directly coupled to terminal 1506 and the electrode.

Because one of the reference voltage signals generated by the DAC is used to drive the electrode in this configuration, this configuration is typically more effective when the number of electrodes that are individually driven in this manner, i.e., the number of peripheral circuits 908 that are configured for operation in this mode at the same time is relatively modest. If the number of such PCs is too large, the DAC may be unable to generate a sufficient number of reference voltage signals to drive all electrodes.

In a fifth configuration, peripheral circuit 908 can operate in a buffer voltage drive mode. Either reference voltage Vₛ₁ or reference voltage Vₛ₂ is buffered through amplifier 1538, and the amplified signal drives the electrode connected to terminal 1506. In this mode, switches s1 (for driving with Vₛ₁) or s2 (for driving with Vₛ₂), s4, and T1 are closed, and the other switches are open. FIG. 16E is a schematic diagram of peripheral circuit 908 operating in this manner (driving with buffered reference voltage Vₛ₁), with active circuit paths shown in heavy black lines.

As a result of this configuration, terminal 1510 is directly connected to terminal 1506. The buffered reference voltage signal connected to the first terminal of amplifier 1538 is routed to terminal 1506 to drive the electrode. This operating mode is effective when the number of PCs configured in this mode is relatively large, e.g., to drive a relatively large number of electrodes. Reference voltage signals generated by the DAC can be amplified via amplifier 1538, generating driving signals for each of the electrodes.

In a sixth configuration, peripheral circuit 908 can operate in a buffer calibration mode. In this mode, reference voltage Vₛ₁ or reference voltage Vₛ₂ is used to calibrate the signal pathway from the DAC to the peripheral circuit to the electrode to identify systemic sources of measurement error. In this mode, switch s1 (for reference voltage Vₛ₁) or s2 (for reference voltage Vₛ₂) and T1 are closed and the other switches are open. FIG. 16F is a schematic diagram of peripheral circuit 908 operating in this manner (calibration with reference voltage Vₛ₁), with active circuit paths shown in heavy black lines. The reference voltage is buffered through amplifier 1538 and read out at terminal 1510 for calibration purposes.

It should be noted that the peripheral circuit shown in FIGS. 15 and 16A-16F is only an example of a peripheral circuit, and more generally, a wide variety of different circuits can be used to detect measurement voltages at electrodes of integrated circuit 102. Further, in addition to the components shown in these figures, peripheral circuit 908 can also include additional circuit components not shown in the figures; and one or more of the components shown in the figures may not be present in certain implementations of peripheral circuit 908.

In addition, the configurations and operating modes described above are only examples. Peripheral circuits 908 present in integrated circuit 102 can be capable of operation in other modes as well, and can have operating configurations that differ from the examples described above. The integrated circuits 102 described herein are not limited to the specific examples of peripheral circuits 908 discussed in this section for illustrative purposes.

### (e) Digital-to-Analog Converters

In some embodiments, integrated circuit 102 includes one or more digital-to-analog converters (DACs). In general, the DAC(s) function to generate a variety of different voltage and/or current signals which are then transmitted to other components of the integrated circuit. Such signals can include, for example, voltage signals that are used to drive electrodes and apply a variety of electrical stimuli to induce responses in a sample positioned in proximity to the electrodes of the integrated circuit. Voltage signals can also include reference voltages that are used to bias measurement signals for detection, and to bias electrodes of the integrated circuit to control the spatial distribution of electric fields in the vicinity of a sample during application of electrical stimuli to the sample.

In general, the number of DACs in each integrated circuit 102 can be independently selected. In certain embodiments, each integrated circuit 102 of the measurement device includes the same number of DACs. In some embodiments, one or more integrated circuits of a measurement device can have a different number of DACs than another one or more integrated circuits of the measurement device. The number of DACs in any integrated circuit 102 can be one or more (e.g., two or more, three or more, four or more, five or more, six or more, eight or more, 10 or more, 15 or more, 20 or more, or even more).

In certain embodiments, each functional group in an integrated circuit 102 has a DAC dedicated to the functional group. Alternatively, in some embodiments, at least one or more DACs are shared by multiple functional groups of the integrated circuit. For example, one or more DACs can be shared by two or more (e.g., three or more, four or more, five or more, six or more, or even more) functional groups within the integrated circuit. In certain embodiments, all functional groups of an integrated circuit share a common DAC.

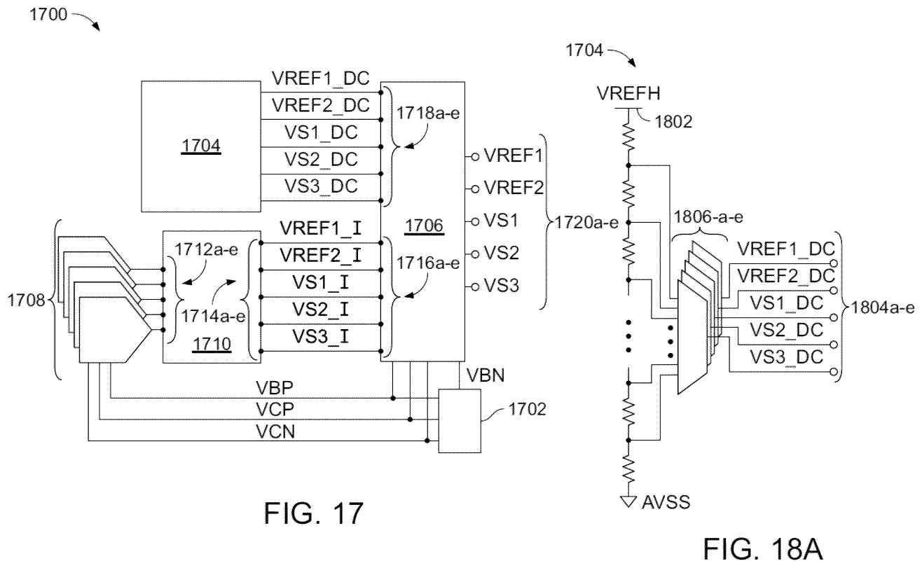

FIG. 17 is a schematic diagram of an example DAC 1700 of an integrated circuit 102. DAC 1700 includes five functional blocks: a voltage bias block 1702, a resistive string module 1704, a trans-impedance amplifier (TIA) bank 1706, a current mode DAC module block 1708, and a switch matrix 1710.

Voltage bias block 1702 generates bias voltage signals V_{BP}, V_{CP}, and V_{CN}. These voltage signals are transmitted to the current mode DAC module block 1708 as shown in FIG. 17 and also transmitted to trans-impedance amplifier bank 1706.

The current mode DAC module block 1708 has multiple input terminals (not shown in FIG. 17), and receives one or more control signals on the input terminals. Block 1708 contains five DAC modules, each of which generates an output signal at an output terminal of the DAC module (not shown in FIG. 17). The output signals are transmitted to the input terminals 1712a-1712e of switch matrix 1710.

Switch matrix 1710 is a 5-to-5 switch matrix, which allows any of the current signals received at input terminals 1712a-1712e to be switched to any of the output terminals 1714a-1714e. Thus, the output signals generated by the multiple DAC modules of DAC module block 1708 can be combined additively at any one or more of output terminals 1714a-1714e.

Switch matrix 1710 is configured to generate five output current signals at terminals 1714a-1714e, respectively, as shown in FIG. 17: V_{REF1_I}, V_{REF2_I}, V_{s1_I}, V_{s2_I}, and V_{s3_I}. These current signals are used to generate corresponding reference voltage signals V_{REF1}, V_{REF2}, Vₛ₁, Vₛ₂, and Vₛ₃ in TIA bank 1706. Specifically, the five output signals from switch matrix 1710 are transmitted to input terminals 1716a-1716e of TIA bank 1706. TIA bank 1706 also receives, at terminals 1718a-1718e, respectively, five DC input voltage signals that correspond to the five reference voltage signals that DAC 1700 generates: V_{REF1_DC}, V_{REF2_DC}, V_{s1_DC}, V_{s2_DC}, and V_{s3_DC}. At each of the five output terminals 1720a-1720e, TIA bank 1706 generates a corresponding output signal that is a reference voltage signal based on the input signals received by TIA bank 1706 at the counterpart input terminals. Thus, for example, at output terminal 1720a, TIA bank 1706 generates an output signal V_{REF1} based on the input signal V_{REF_DC} at input terminal 1718a and the input signal V_{REF1_I} at input terminal 1716a. The output voltage signal at terminal 1720 is V_{REF1}=V_{REF1_DC}+R*V_{REF1_I}, where R is the gain factor for TIA bank 1706 for the signal at output terminal 1720a. Output signals at terminals 1720b-1720e are generated analogously, and are shown in FIG. 17.

Terminals 1720a and 1720b supply reference voltage signals V_{REF1} and V_{REF2} either directly or indirectly (i.e., through other circuit elements) to the pixel circuits of integrated circuit 102 described above. In some embodiments, for example, terminals 1720a and 1720b are connected to other components of integrated circuit 102, such as peripheral circuits and/or reference electrode amplifiers, which use these voltage signals as bias voltages in operations involving the driving of electrodes with voltage and/or current mode signals, and detecting measurement signals at the electrodes of the integrated circuit.

Terminals 1720c-1720e are connected to the peripheral circuits 908 of integrated circuit 102, so that reference voltage signals Vₛ₁, Vₛ₂, and Vₛ₃ are transmitted to the peripheral circuits as described above.

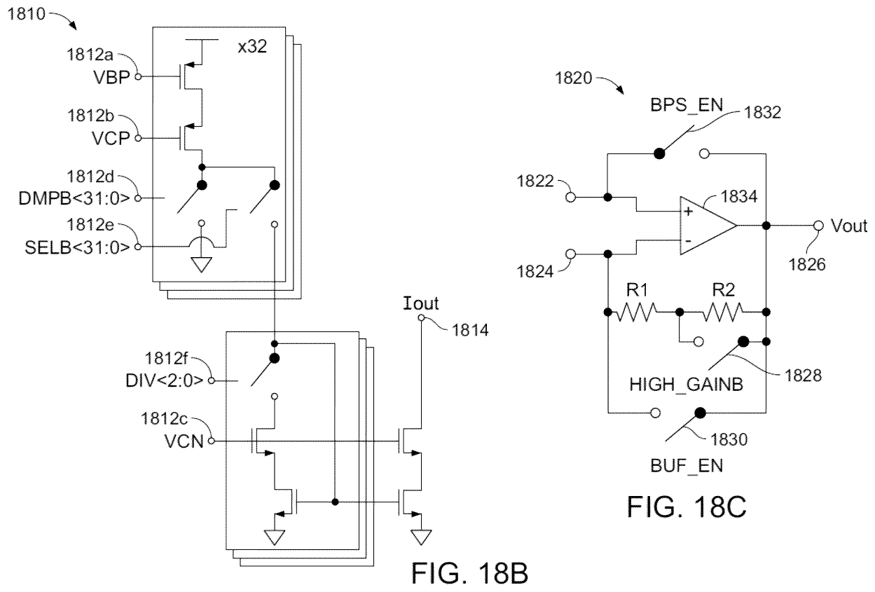

As shown in FIG. 17, resistive string module 1704 functions to generate five DC reference voltages that are transmitted to TIA bank 1706. FIG. 18A is a schematic diagram showing an example of resistive string module 1704. Module 1704 includes an input terminal 1802 and five output terminals 1804a-1804e. Resistors of module 1704 are connected in single series string, and five analog multiplexers 1806a-1806e are connected to the resistor string at different tap locations.

Module 1704 receives an input voltage signal V_{REF}H at terminal 1802 from an external source (i.e., from another source located external to integrated circuit 102). Each analog multiplexer is connected to the resistor string at 31 different tap locations via 31 input terminals, and can couple its output terminal to the input signal received at any one of the 31 input terminals to generate a corresponding output signal. In this manner, each of the different DC reference voltages V_{REF1_DC}, V_{REF2_DC}, V_{s1_DC}, V_{s2_DC}, and V_{s3_DC} is generated by one of the analog multiplexers 1806a-1806e and transmitted to a corresponding output terminal 1804a-1804e of resistive string module 1704.

As discussed above, DAC module block 1708 contains multiple DAC modules, each of which functions as a current signal source. In some embodiments, for example, DAC module block 1708 includes 32 unit current sources. More generally, however, DAC module block 1708 can include any number of current sources (e.g., five or more, 10 or more, 15 or more, 20 or more, 25 or more, 30 or more, 35 or more, 40 or more, 50 or more, or even more).

FIG. 18B is a schematic diagram of an example current source 1810. Current source 1810 includes input terminals 1812a-1812f and output terminal 1814. At input terminals 1812a-1812c, current source 1810 receives bias voltage signals V_{BP}, V_{CP}, and V_{CN} from voltage bias block 1702. At input terminals 1812d-1812f, current source 1810 receives digital control bit signals (e.g., from host controller 706) that control the operation of the current source.

Current source 1810 is inactive and draws no power when digital control bits DMPB and SELB are inactive. When DMPB is asserted and SELB is inactive, current source 1810 is on, but does not generate an output current signal. When SELB is also asserted, current source 1810 generates an output current signal at terminal 1814.

The output current signal at terminal 1814, I_{OUT}, is a function of the input current signal at terminals 1812a-1812b determined by bias voltages V_{BP} and V_{CP}, respectively, by the number of asserted SELB signals s among the current sources of DAC module block 1708, and by the gain factor G of the current source. The gain factor G of the current source is controlled by a 3-bit digital control signal received by current source at terminal 1812f, DIV. The output current signal is expressed as I_{OUT}=s*G*I_{REF}. Values of the digital control signal DIV and the corresponding gain factor G are shown in Table 3 below.

TABLE 3

| DIV Control Signal | Gain Factor G |
|---|---|
| 000 | 0 |
| 001 | 1 |
| 010 | 1/2 |
| 011 | 1/3 |
| 100 | 1/4 |
| 101 | 1/5 |
| 110 | 1/6 |
| 111 | 1/7 |

Output current signals from the output terminals 1814 of the current sources are transmitted to input terminals 1712a-1712e of switch matrix 1710, as described above.

TIA bank 1806 includes multiple TIAs, each of which is typically configured to generate an output voltage signal. In general, TIA bank 1806 can include any number of TIAs (e.g., one or more, two or more, three or more, five or more, seven or more, 10 or more, 15 or more, 20 or more, or even more). In the example shown in FIG. 17, TIA bank 1806 includes five TIAs, each of which is configured to generate one of the output voltage signals at terminals 1720a-1720e.

FIG. 18C is a schematic diagram of an example TIA 1820. TIA 1820 includes input terminals 1822 and 1824, output terminal 1826, switches 1828, 1830, and 1832, and operational amplifier 1834. At input terminal 1822, TIA 1820 receives one of the DC reference voltage signals generated by the resistive string module 1704. Input terminal 1822 corresponds to, or is connected to, one of terminals 1718a-1718e. At input terminal 1824, TIA 1820 receives one of the current mode signals generated by switch matrix 1710. Input terminal 1824 corresponds to, or is connected to, one of terminals 1716a-1716e.

TIA 1820 receives a digital control bit signal AMP_EN at a terminal (not shown in FIG. 18C) that controls the on/off state of TIA 1820. When AMP_EN is asserted, TIA 1820 is on, and when AMP_EN is not asserted, TIA 1820 is off. In its off state, TIA 1820 draws very low (or no) power and provides a high impedance at terminal 1826, ensuring that it is bypassed in this state.

When TIA 1820 is on, it can operate in different operating modes depending upon the state of switches 1828, 1830, and 1832. Table 4 shows various operating modes for TIA 1820 as a function of the state of switches 1828 (HIGH_GAINB), 1830 (BUF_EN), and 1832 (BPS_EN).

TABLE 4

| | | | | | Terminal | Terminal |
|---|---|---|---|---|---|---|
| Operating Mode | BPS_EN | HIGH_GAINB | BUF_EN | AMP_EN | 1822 | 1824 |
| Off | Open | Open | Open | 0 | X | X |
| Bypass | Closed | Open | Open | 1 | Input | X |
| Buffer | Open | Closed | Closed | 1 | Input | X |
| TIA low gain | Open | Closed | Open | 1 | Input | input |
| TIA high gain | Open | Open | Open | 1 | Input | input |

In Bypass mode, the DC voltage signal generated by the resistor string module and received at terminal 1822 is transmitted directly to output terminal 1826. The resulting output reference voltage signal has relatively low noise compared with an amplified output signal, but comparatively higher output impedance.

In Buffer mode, the DC voltage signal generated by the resistor string module is buffered by amplifier 1834 operating as a unity gain buffer. The buffered DC voltage signal is transmitted to output terminal 1826.

In the TIA modes, the output voltage is a function of resistances R1 and R2, which are selectively coupled into TIA 1820 depending upon the state of switches 1828 and 1830. In TIA low gain mode, the output voltage at terminal 1826 is V_{OUT}=V_{IN}+R1*I_{IN}, where V_{IN} is the input reference voltage signal at terminal 1822, and I_{IN} is the current input signal at terminal 1824. In TIA high gain mode, the output voltage at terminal 1826 is V_{OUT}=V_{IN}+(R1+R2)*I_{IN}.

An important aspect of DAC 1700 involves the generation of multi-frequency output voltage signals. Multi-frequency output voltage signals can be generated in different ways using DAC 1700. In a first mode (“5-bit mode”), output voltage signals are generated from analog current signals and combined in analog form to generate a multi-frequency output voltage signal.

Referring again to FIG. 18B, in this mode, one current source 1810 is used to generate each analog current signal at a particular frequency. Thus, for a three-frequency output voltage signal, three different current sources 1810 are used. The first current source 1810 generates an analog current signal at its output terminal 1814 with a frequency f1. The second current source 1810 generates an analog current signal at its output terminal 1814 with a frequency f2. The third current source 1810 generates an analog current signal at its output terminal 1814 with a frequency f3. Each of the frequencies f1, f2, and f3 are different.

Each of the analog output current signals from the three current sources 1810 is then combined by switch matrix 1710 at a single output terminal of the switch matrix to generate an output current signal containing the three frequency components, f₁, f₂, and f₃. For example, to generate an output signal V_{REF1} containing the three frequency components, the three analog current signals at frequencies f₁, f₂, and f₃, respectively, are routed to terminal 1714a of switch matrix 1710. Effectively, the three analog current signals are added by switch matrix 1710 in the current domain, and the resulting signal is provided at terminal 1714a. The output analog current signal containing frequency components f₁, f₂, and f₃ is routed to terminal 1716a of TIA bank 1706.

The amplitude profile of the current signal generated by each current source 1810 is controlled through configuration bits transmitted to the current source. The configuration bits define the amplitude profile of the current signal through one cycle of the current signal. Thus, for example, a current signal with a sine wave amplitude profile can readily be generated by setting configuration bit values to define the amplitude profile of a sine wave. More generally, each current source 1810 can be configured to generate an output current signal with a wide variety of different amplitude profiles by transmitting appropriate corresponding configuration bit values to each current source. Suitable configuration bit values are determined and transmitted, e.g., by host controller 706. The configuration bit values can also be stored in a memory unit (not shown in FIG. 18B) and read from the memory unit by current source 1810 to set the amplitude profile of the output current signal.

The frequency of the current signal generated by each current source is determined by a clocking signal transmitted to each current source 1810. The clocking signal controls the frequency at which the current source generates one cycle of an output current signal having the amplitude profile defined by the configuration bit values. To generate an output current signal at a frequency f1, for example, the frequency of the clocking signal is set to f1 (e.g., by host controller 706), and the current source generates cycles of the output current signal at a frequency f1.

The foregoing method of generating an output current signal at a desired frequency can be advantageous, as it allows output current signals to be generated at a wide range of frequencies simply by adjusting the frequency of the clock signal transmitted to each current source 1810. Further, it allows for the generation of current signals with a wide variety of amplitude profiles, and the amplitude profile of the output current signal can be adjusted independently of the frequency of the output current signal.

It should be noted that each current source 1810 can be adjusted independently with respect to both configuration bit values defining the output current signal amplitude profile, and clocking frequency. During operation, current sources 1810 can be clocked at the same frequency and phase, or at different frequencies, different phases, or both different frequencies and phases. Further, current sources 1810 can be configured to generate output current signals with different amplitude profiles or the same amplitude profile. Still further, current sources 1810 can be configured to use the same number or different numbers of configuration bits to define the amplitude profile. In some embodiments, for example, the number of configuration bits used to define the amplitude profile of each output current waveform can be adjusted based on the nature of the output current waveform that is desired.

As discussed above in connection with DAC control block 1708, the 3-bit digital control signal DIV for each current source controls the gain factor of the source. The DIV control signals of each of the current sources used to synthesize the output signal at terminal 1714a can be adjusted relative to one another to individually weight the amplitudes of the frequency components of the output signal at terminal 1714a. The configuration bit values used to define the amplitude profile of the output current waveform generated by each current source 1810 can also be adjusted (relative to the configuration bit values for the other current sources 1810) to effectively weight the output current signals generated relative to one another prior to combining the signals. As noted above, the relative phases of the clocking signals transmitted to each current source 1810 can also be controlled; by doing so, the relative phases of the frequency components in the output current signal at terminal 1714a can be controlled.

In a second mode (“7-bit mode”), the output terminals 1814 of 4 current sources 1810 are connected to the same input terminal (and therefore the same TIA 1820) of TIA bank 1706 by switch matrix 1710. For example, the output terminals of 4 current sources can be connected to terminal 1714a (to generate an output signal V_{REF_I} with multiple frequency components) or to terminal 1714d (to generate an output signal V_{s2_I} with multiple frequency components). Connected in parallel in this manner, the 4 current sources effectively form a 7-bit current source.

In this mode, the multi-frequency output current signal is generated digitally by current sources. The configuration bit values for each current source 1810 define an amplitude profile that corresponds to a cycle (or a portion of a cycle) of a multi-frequency output current signal, and is therefore typically more complex than the single frequency amplitude waveforms discussed above. Each current source 1810 receives a clocking signal as described above, and generates repeated cycles (or portions of cycles) of the multi-frequency output current signal. The multi-frequency signals generated by each of the current sources 1810 are then combined additively by switch matrix 1710 and provided at a common output terminal of the switch matrix. The additive combination of the multi-frequency signals generated by each of the current sources 1810 yields an output current signal containing multiple frequencies. However, contrary to the first mode of operation described previously, in this mode the frequency components are effectively added digitally by configuring each of the current sources 1810 with appropriate configuration bit values to define multi-frequency amplitude profiles for the output current signals.

### (f) Measurement Signals and Data Communication

Measurement signals at electrodes in each integrated circuit 102 are detected by peripheral circuits 908 as described above. The measurement signals, which detected as analog signals, are then converted to digital signals by ADCs 904. However, due to the relatively large number of electrodes in each integrated circuit, it can be challenging to detect measurement signals from each of the electrodes of each of the integrated circuits of a measurement device, and transmit all of the detected signals to an external device, e.g., host controller 706, in a power efficient manner, while maintaining a high data throughput rate to ensure that measurements can be performed across all integrated circuits of the measurement device within reasonable and relevant time intervals.

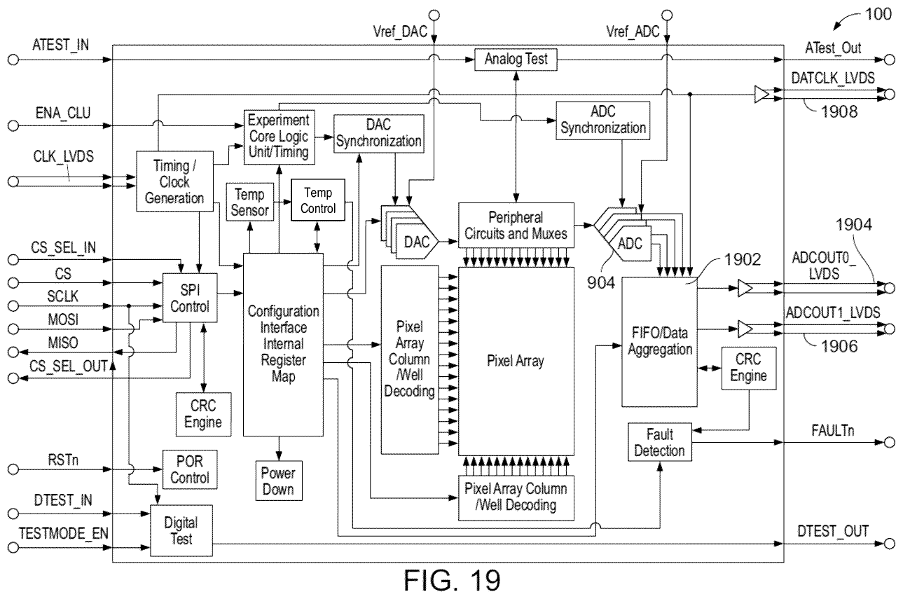

To achieve relatively high data throughput rates at modest power consumption, the measurement devices described herein, in some embodiments, include a low voltage differential signaling (LVDS) interface for communication of detected measurement signals. FIG. 19 is a schematic diagram showing an example of an integrated circuit 102 that includes many of the features described above. As shown in FIG. 19, after conversion to digital signals ADCs 904, the measurement signals are routed to and buffered by a data aggregation unit 1902.

Measurement device 100 in FIG. 19 includes two low voltage differential signaling (LVDS) data output lines 1904 and 1906 for transmitting digital measurement signals to an external device such as host controller 706. The LVDS output lines operate in parallel according to a first-in, first-out (FIFO) data transport scheme.

In the example shown in FIG. 19, each ADC 904 is connected to its own independent FIFO buffer within data aggregation unit 1902. Signals routed from the ADCs to the corresponding buffers of data aggregation unit 1902 are then transmitted sequentially in order on the LVDS output lines, according to the number of ADCs that are enabled. Thus, for example, with signals provided by 8 ADCs to data aggregation unit 1902, the data aggregation unit 1902 cycles through each of the ADC FIFO buffers in turn, transmitting the digital signals contained in each buffer in order. In the event a particular ADC is disabled and is not generating data signals, the data aggregation unit bypasses the FIFO buffer associated with the disabled ADC.

Typically, each ADC 904 receives the same clocking signal, and therefore the ADCs nominally operate in synchronous fashion. However, in practice, finite timing differences may exist between the ADCs, with the result that data from a particular enabled ADC may not be available in its corresponding FIFO buffer when data aggregation unit 1902 samples the buffer for data transmission on the LVDS output lines. In this event, the data aggregation unit will wait until data is received in the FIFO buffer, transmit the received data on the LVDS output lines, and then move on to the next FIFO buffer in sequence.

Each LVDS output line can be independently enabled or disabled by a control signal, e.g., from host controller 706. For example, for measurements in which the number of measurement signals is not as high as in other measurements, data corresponding to the measurement signals can be transmitted at acceptable rates by only one of the LVDS output lines. In these circumstances, the other LVDS output line can be disabled to reduce power consumption by measurement device 100.

In addition to the two LVDS data output lines, the LVDS interface includes a LVDS clock channel 1908. The clock channel transmits a clock signal that is synchronized to the data stream on the LVDS data output lines. Because the data streams on the data output lines contain measurement signals interleaved from multiple integrated circuits 102, the external device that receives the data streams on the LVDS data output lines can de-multiplex the data streams based on the transmitted clock signals, and attribute the measurement signals of the data streams to specific electrodes of specific integrated circuits of the measurement device.

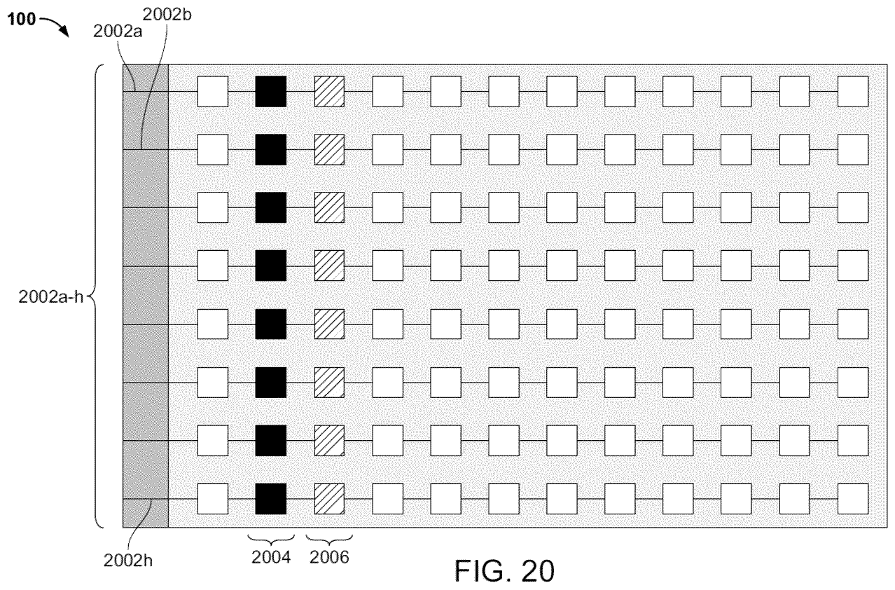

To simplify connections between the integrated circuits 102 and the LVDS interface, in some embodiments, multiple integrated circuits 102 of a measurement device share a connection to the LVDS interface. FIG. 20 is a schematic diagram showing a measurement device 100 with a two-dimensional arrangement of integrated circuits 102 on the device. Measurement device 100 includes LVDS connection lines 2002a-2002h, each of which is connected to the LVDS data output lines 1904 and 1906. As shown in FIG. 20, each of the integrated circuits 102 in a row of the measurement device shares a LVDS connection line 2002a-2002h.

To offload detected measurement signals from the integrated circuits 102 of measurement device 100, only one of the integrated circuits within each row of FIG. 20 is enabled for data transmission at a time. In FIG. 20, the integrated circuits of column 2004 are enabled for data transmission, and measurement signals detected from the electrodes of each of the enabled integrated circuits are transmitted to LVDS data output lines 1904 and 1906 via LVDS connection lines 2002a-2002h. For each of the LVDS connection lines, only one integrated circuit sharing the connection line is active and transmitting data. Thus, none of the LVDS connection lines transmits data from multiple integrated circuits at the same time in a common data stream.

While the integrated circuits of column 2004 transmit detected measurement signals, the integrated circuits of column 2006 can optionally be prepared for data transmission, e.g., by configuring various attributes of the electrodes and other components of these integrated circuits. By pre-configuring integrated circuits in this manner, delays associated with transmitting detected measurement signals from different integrated circuits of the measurement device can be reduced or even minimized. As will be explained in more detail subsequently, a different interface independent of the LVDS output lines can be used to prepare the integrated circuits of column 2006 for data transmission, so that the LVDS output lines remain dedicated to data transmission from the ADCs to an external host, e.g., host controller 706.

It should be appreciated that the enabling and disabling procedure shown in FIG. 20 is merely an example. A wide variety of different procedures can be implemented. For example, the integrated circuits that actively transmit detected measurement signals on connection lines 2002a-2002h do not need to be aligned in a common row or column on the measurement device. Any integrated circuit on any of the connection lines 2002a-2002h can be configured to transmit detected measurement signals at a particular time. Further, while the configuration shown in FIG. 20 uses one shared LVDS connection line for each row of integrated circuits, more generally, each LVDS connection line can be shared among integrated circuits that are positioned at any location of the measurement device. The integrated circuits that share a particular LVDS connection line do not need to be aligned in a row or column. In some embodiments, within a row or column of integrated circuits, some integrated circuits may share one LVDS connection line, and some integrated circuits may share another LVDS connection line. In that context, the number of LVDS communication lines in each measurement device can be two or more (e.g., three or more, four or more, eight or more, 16 or more, 32 or more, 64 or more, or even more). Moreover, each LVDS communication line can be shared among two or more (e.g., three or more, four or more, five or more, six or more, seven or more, eight or more, 10 or more, 15 or more, 20 or more, 30 or more, 40 or more, 50 or more, or even more) integrated circuits of a measurement device.

Further, it should be noted that the enabling and disabling procedure described above in connection with FIG. 20 is useful to reduce power consumption by measurement device 100. By disabling integrated circuits from which measurement signals are not being transmitted via the LVDS output lines (and, optionally, integrated circuits that are not being prepared for data transmission), the power consumption of measurement device 100 can be reduced. When measuring the responses of certain biological samples to particular stimuli, the effective sampling rate that results from the enabling and disabling procedure described above is adequate to capture relevant biological responses.

However, if higher sampling rates are desirable, e.g., when biological responses to electrical stimuli occur more quickly, measurement device 100 can include more LVDS connection lines than the number of LVDS connection lines (e.g., 2000a-h) shown in FIG. 20. That is, the number of integrated circuits that share a common LVDS connection line can be reduced to allow for higher data transfer rates via the LVDS data output lines. In general, the number of integrated circuits that share a common LVDS connection line can be one or more (e.g., two or more, three or more, four or more, five or more, six or more, seven or more, eight or more, nine or more, 10 or more, 12 or more, 15 or more, 20 or more, 30 or more, or even more). In some embodiments, to achieve very high data transfer rates, each integrated circuit has a dedicated LVDS connection line that is not shared with other integrated circuits of measurement device 100.

The high-throughput LVDS data output lines 1904 and 1906 of measurement device 100 allow detected measurement signals to be transmitted from each integrated circuit to an external device such as host controller 706 without on-chip data buffering. Disabling integrated circuits that are not transmitting detected measurement signals can also yield a significant reduction in heat generation and power consumption. Because excessive heating can cause adverse consequences for living biological samples such as cells, reduction of anomalous heating can be an important advantage of the data offloading procedure described above.

Returning to FIG. 19, the data aggregation unit 1902 (which can be part of integrated circuit 102 in some embodiments) includes FIFO buffers that receive detected measurement signals from the ADCs of the integrated circuit, align the signals to the LVDS clock signal, and pack data streams to match a data transmission rate of 1 or 2 LVDS data output lines. If the rate at which detected measurement signals from electrodes of the integrated circuits that are actively being scanned is sufficiently low such that the data rate (in bits per unit time) of the data streams from the integrated circuits is within the data transmission rate capacity of a single LVDS data output line, then the measurement device—responsive to a control signal from, e.g., host controller 706—can de-activate one of the LVDS data output lines. Doing so can result in significant reductions in power consumption and heat generation by the measurement device.

In some embodiments, the LVDS interface (and specifically, the LVDS data output lines) in measurement device 100 can support dual data rate (DDR) transmission of data corresponding to detected measurement signals from integrated circuits of the device. In single data rate (SDR) operation, data is transmitted on either the rising or falling edge of the LVDS clock signal. As discussed previously, the external device that receives the transmitted data stream can de-multiplex the data stream based on the rising or falling edges of the LVDS clock signal. In DDR operation, data corresponding to detected measurement signals is transmitted on both the rising and falling edges of the LVDS clock signal. As a result, the data transmission rate is effectively doubled, and the increase in power consumption associated with DDR operation compared to SDR operation is relatively modest. For high data rate transmission, operation in DDR mode further improves power utilization efficiency.

### III. Stimulation and Measurement Methods and Operations

The measurement devices and systems described above can be used to perform a wide variety of different types of measurements on biological samples. In general, measurements involve providing one or more electrical stimuli to the sample, and then measuring responses of the sample to the electrical stimuli. Typically, measurements are performed in parallel on multiple samples positioned on or adjacent to a measurement device. In some embodiments, the same stimulus is provided to all of the samples positioned on or adjacent to a measurement device; alternatively, in certain embodiments, different stimuli are provided to different samples. Similarly, in certain embodiments, the measurement of sample responses to the provided stimulus is performed in the same manner for all samples; alternatively, in some embodiments, the measurement of sample response to the provided stimulus is different for different samples.

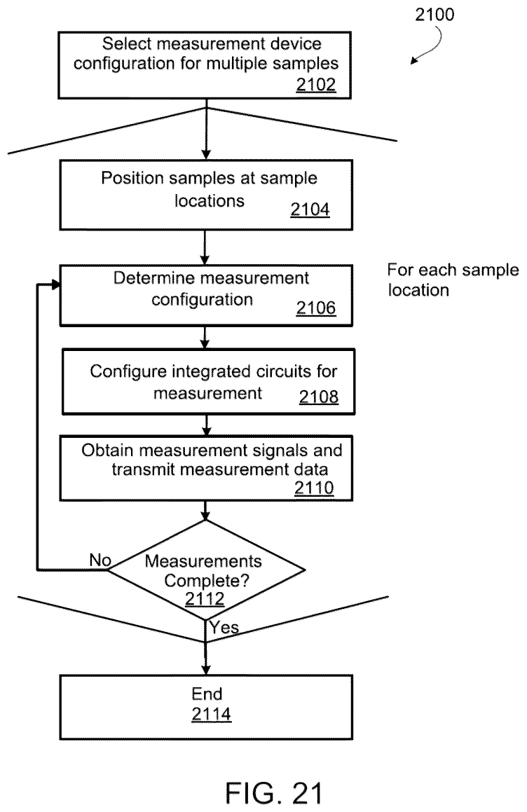

FIG. 21 is a flow chart 2100 that shows a series of example steps that can be performed to measure responses to electrical stimuli from a one or more samples. In the following discussion, each of the steps shown in FIG. 21 will be explained in detail. However, it should be understood that the flow chart shown in FIG. 21 and the following discussion is provided by way of example only, and that the measurement devices and systems are not limited to only performing measurements in the described manner. More generally, measurements can be performed as desired using the measurement devices and systems described herein, with modifications to any of the steps shown in FIG. 21.

In a first step 2102 of flow chart 2100, a measurement device configuration for multiple samples is selected. As explained above in connection with FIGS. 3-6, selecting the measurement device configuration involves selecting the number of samples that will be positioned on or adjacent to measurement device 100 and how the samples will be positioned relative to integrated circuits 102 for measurement purposes. In some embodiments, selecting the measurement device configuration involves overlaying an enclosure 302 on substrate 104 of measurement device 100 to define multiple wells into which samples can be placed for measurement purposes. More generally, whether wells are defined on measurement device 100 or not, selecting the measurement device configuration includes selecting sample locations on measurement device 100. Typically, when wells are defined on measurement device 100, the sample locations correspond to the locations of the wells. However, even in the absence of wells, a plurality of sample locations can be defined simply as areas on measurement device 100 where samples will be positioned.

At each sample location, at least a portion of the electrodes 902 of the integrated circuit 102 associated with the sample location are allocated to providing electrical stimuli to a sample at that location and measuring responses of the sample to the provided stimuli. In some embodiments, as discussed in connection with FIG. 3, there is 1:1 relationship between the number of integrated circuits 102 in a measurement device and the number of sample locations on the measurement device, such that one integrated circuit is associated with each sample location. In this configuration, all of the electrodes of the integrated circuit 102 are allocated to measurements for the sample at the location associated with the integrated circuit. However, in certain embodiments, as discussed in connection with FIGS. 4 and 5A-5C, one integrated circuit is associated with multiple sample locations. In these circumstances, the electrodes 902 of an integrated circuit 102 associated with multiple sample locations are partitioned into groups, with each group of electrodes allocated to perform measurements in one of the sample locations, i.e., on one of the samples. As explained previously, in certain embodiments, the electrodes 902 of a particular integrated circuit 102 can generally be divided into any number of groups. FIGS. 5B and 5C show specific examples in which the electrodes of an integrated circuit have been divided into four groups (FIG. 5B) and sixteen groups (FIG. 5C) by establishing four and sixteen sample locations associated with integrated circuits 102, e.g., by defining wells using enclosure 302.

In the discussion that follows, each of the subsequent steps of flow chart 2100 are performed at each sample location on measurement device 100. To simplify the explanation, the specific example in which there is a 1:1 relationship between sample locations and integrated circuits is discussed. That is, each sample location is associated with one integrated circuit 102, and the integrated circuit's entire complement of electrodes 902 are allocated to measurements involving the sample at that location. However, it should be understood that in methods where multiple sample locations are associated with individual integrated circuits 102, similar method steps are performed at each sample location, and the electrodes of an integrated circuit 102 associated with multiple sample locations are partitioned into groups associated with the multiple sample locations, with one group of electrodes allocated to each sample location. Thereafter, method steps specific to the multiple sample locations are performed only with the group of electrodes allocated to those locations.

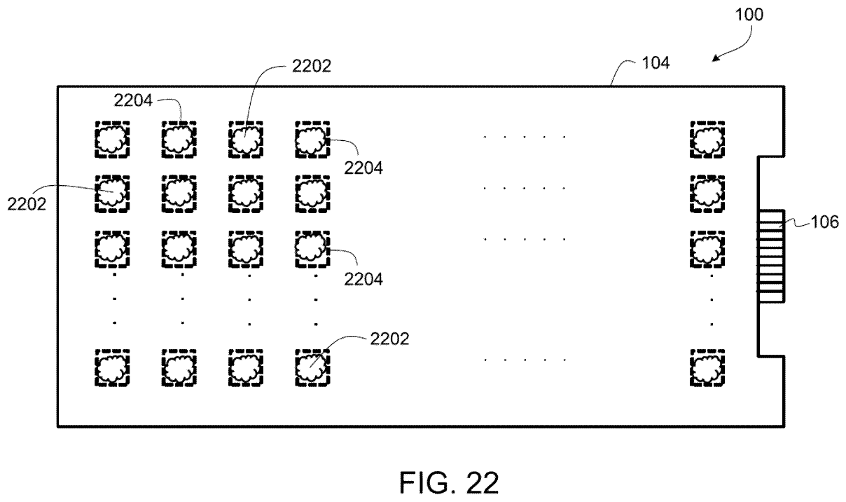

With the measurement configuration selected and sample locations on or adjacent to measurement device 100 determined in step 2102 of flow chart 2100, in the next step 2104, samples are positioned at each of the sample locations. FIG. 22 is a schematic diagram showing an example of the results of this positioning step. In FIG. 22, samples 2202 are positioned at each of the sample locations 2204 defined on the surface of substrate 104 of measurement device 100. As noted above, in this example, each of the sample locations 2204 is associated with one of the integrated circuits 102 of measurement device 100, and thus, an integrated circuit 102 underlies each of the locations on substrate 104 at which a sample 2202 is positioned.

In general, the samples 2202 positioned at each of the sample locations 2204 include cells, and typically, living cells. In some embodiments, single cells are positioned at one or more sample locations 2202, and in certain embodiments, each of the sample locations can be occupied by only a single cell. In some embodiments, some or all of the sample locations can be occupied by multiple cells. Cells can generally be obtained using a wide variety of methods, including growth in a culture medium and extraction from a living subject (such as via biopsy); in general, the methods described herein can use any technique for acquiring samples of cells.

Moreover, the cells can generally be of any type. Cells can, for example, be cells of various organs, including (but not limited to) liver cells, kidney cells, lung cells, heart cells, and skin cells. Cells can also be circulating cells found in bodily fluids such as blood, saliva, lymph, and other bodily fluids. Cells can also be cells of non-organ tissues such as muscle cells, fat cells, and cells of connective tissues. Cells can be associated with various disease conditions, such as tumor cells, viral host cells, and bacterial cells.

After cellular samples have been obtained, they can be positioned at each of the sample locations 2204 using a variety of methods. In some embodiments, for example, cells are deposited at the sample locations using pipetting and/or other microfluidic methods. Such methods can be performed manually, or in automated fashion, e.g., by a robotic sample dispensing assembly.

Returning to FIG. 21, in the next step 2106, at each sample location, a measurement configuration is determined. Determining the measurement configuration involves selecting the type of measurement that will be performed and the electrode configuration used to perform the measurement. In general, for the electrodes associated with each sample location, this involves selecting:
(a) the type of electrical stimulus that will be provided to the sample;
(b) the locations at which the electrical stimulus will be provided to the sample;
(c) the type of measurement signal that will be measured, reflecting the response of the sample to the provided electrical stimulus; and
(d) the locations at which the measurement signal will be measured.

Various types of electrical stimuli can be provided to a sample. In some embodiments, the sample is stimulated by current injection. Current injection includes delivering a low impedance current signal to the sample via one or more electrodes of an integrated circuit. To inject current in this manner, as explained previously, the pixel circuits 1054 connected to the electrodes that inject the current can operate in mode 1 or mode 2.

In certain embodiments, the sample is stimulated with a voltage signal by one or more electrodes of an integrated circuit. To stimulate the sample in this manner, the pixel circuits 1054 connected to the electrodes that provide the voltage signal can operated in one of modes 10 or 12, in which voltages V_{REF1} or V_{REF2} are applied to the electrodes respectively, or in mode 9 with corresponding pixel circuits configured for operation in the fourth configuration (where the electrode is directly driven at voltage Vₛ₁, Vₛ₂, or Vₛ₃). As another alternative, with corresponding pixel circuits configured for operation in the sixth configuration (as shown in FIG. 16F) and switch s4 closed so that terminal 1506 is connected to the negative terminal of amplifier 1538, voltage signals Vₛ₁ (with switch s1 closed) or Vₛ₂ (with switch s2 closed) can be buffered through amplified 1538 and provided to the electrodes through their connected pixel circuits 1054 to stimulate the sample.

In addition to the choice of current and voltage signals for application to the sample, selecting the type of electrical stimulus also involves—for applied oscillating current and voltage signals—selecting the frequency of the stimulation signal. The measurement devices described herein can be used to generate both current and voltage signals at a wide range of frequencies for application to a sample. For example, the frequency of an oscillating current or voltage signal applied to the sample can be between 10 Hz and 1 MHz (e.g., between 50 Hz and 800 kHz, between 100 Hz and 500 kHz, between 200 Hz and 300 kHz, between 500 Hz and 200 kHz, between 1 kHz and 100 kHz, or any range that falls within any of the foregoing ranges). The frequency of an oscillating current or voltage signal applied to the sample can be 10 Hz or more (e.g., 20 Hz or more, 30 Hz or more, 50 Hz or more, 100 Hz or more, 200 Hz or more, 300 Hz or more, 500 Hz or more, 1 kHz or more, 5 kHz or more, 10 kHz or more, 20 kHz or more, 50 kHz or more, 100 kHz or more, 200 kHz or more, 300 kHz or more, 500 kHz or more, or even more). The frequency of an oscillating current or voltage signal applied to the sample can be 1 MHz or less (e.g., 900 kHz or less, 800 kHz or less, 700 kHz or less, 500 kHz or less, 300 kHz or less, 100 kHz or less, 50 kHz or less, 30 kHz or less, 20 kHz or less, 10 kHz or less, 5 kHz or less, 1 kHz or less, 900 Hz or less, 800 Hz or less, 700 Hz or less, 500 Hz or less, 300 Hz or less, 100 Hz or less, or even less).

In addition to single-frequency electrical stimuli, the measurement devices described herein can also be used to generate both current and voltage signals at multiple frequencies. As discussed above, DAC 1700 can be used to generate current and voltage signals that include more than one (e.g., two or more, three or more, four or more, five or more, or even more) frequency components. Each of the frequency components can be within any of the ranges discussed above. Further, the phases of the frequency components can be adjusted relative to one another. In some embodiments, multi-frequency current and voltage signals used for electrical stimulation generally correspond to the superposition of sinusoidally-varying signals at each of the multiple frequencies. However, in some embodiments, more complex amplitude profiles for current and voltage signals used for stimulation can be defined digitally, as described previously in connection with DAC 1700.

The choice of locations at which various electrical stimuli are provided to the sample depends in part on the type of sample response that will be interrogated. In some measurement methods, for example, electrical stimuli are provided by some or all of the electrodes of an integrated circuit according to a spatial pattern, e.g., by every jth electrode of the integrated circuit, where j is an integer. In certain methods, electrical stimuli are provided by all of the electrodes of an integrated circuit.

However, more complex stimulation configurations can also be used in measurements for a variety of reasons. For example, in some measurements, particular stimulation configurations are used to prevent coupling between neighbor electrodes of an integrated circuit, which may lead to leakage currents and other undesirable electrical artifacts in measured sample responses. Further, in some measurements, particular stimulation configurations are used to provide more exacting control over the nature of the stimulus provided to a sample, and to the resulting measurement signals from the sample in response to the provided stimulus.

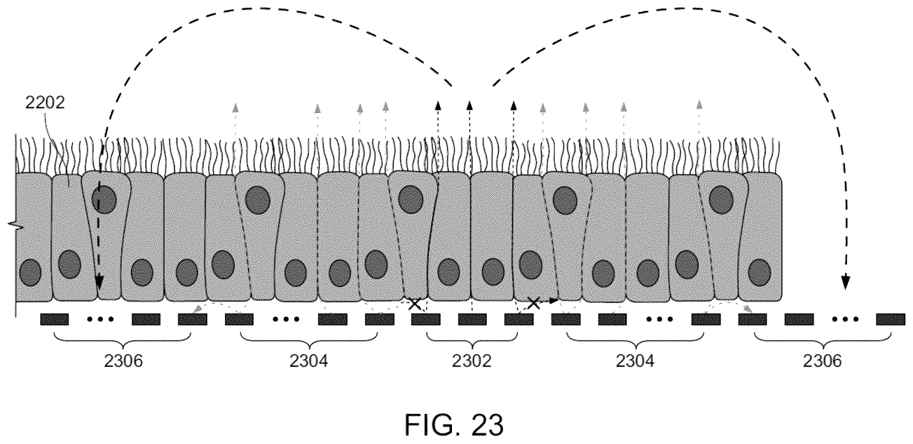

For example, in some embodiments, beyond simple impedance measurements at individual electrodes, the measurement devices described herein can be used to investigate the response of a sample to vertically-oriented electric fields, which is associated with the Transepitheial Electrical Resistance (TEER) value or Barrier Function/Tight-Junction value for the sample. FIG. 23 shows a schematic cross-sectional view of an example of measurement configuration in which the response of a sample to a vertically-oriented electric field is interrogated. In FIG. 23, a first group of electrodes 2302 (9 electrodes in total, only 3 are shown in the cross-sectional view) deliver AC signals to sample 2202. The same group of electrodes 2302 is used to measure current signals from the sample in response to the stimulation. A second group of electrodes 2304 is biased with the same AC signals to create a group of shielding electrodes. Because of this biasing of the second group of electrodes 2304, the electric field generated by the first group of electrodes 2302 is constrained to be oriented vertically as shown by the dotted field lines in FIG. 23. Horizontal components of the electric field generated by the first group of electrodes 2302 are largely eliminated by this biasing configuration, as indicated by the “X”s in FIG. 23. As a result, the current introduced by the first group of electrodes 2302 passes through only the relatively tight junctions at the cell membranes. A third group of electrodes 2306 is biased at a ground potential, and function as return electrodes for the vertically-oriented electric field generated by the first group of electrodes 2302, and also for the shielding electrodes 2304.

In the measurement configuration shown in FIG. 23, it should also be noted that measurement signals are obtained from each of the 9 electrodes of the first electrode group 2302 at effectively the same time. This provides an important multiplex advantage relative to methods in which measurements are obtained from individual electrodes sequentially, and can significantly reduce the amount of time required to measure signals at each of the electrodes of an integrated circuit.

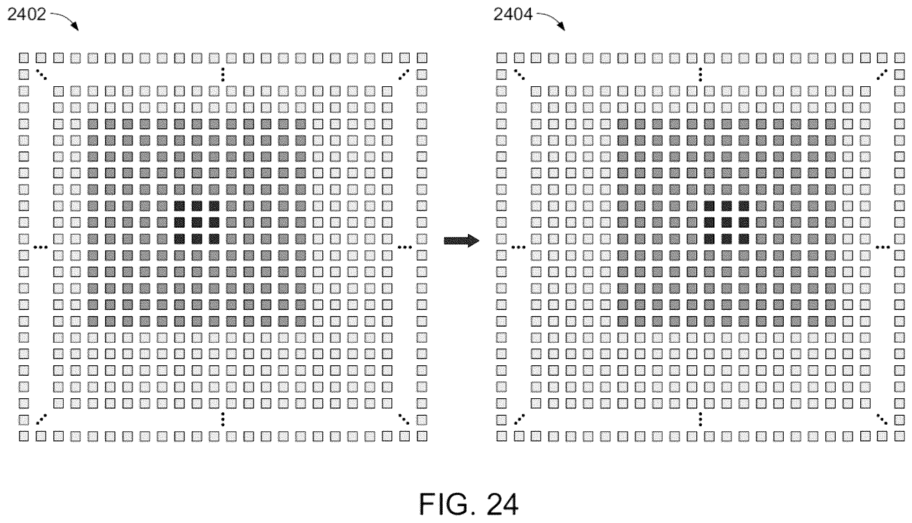

Obtaining measurement signals at all electrodes associated with the sample location in FIG. 23 involves making measurements in “frames”, as discussed previously. In effect, the measurement method involves cycling through all electrodes of an integrated circuit associated with the sample location in groups, such that in each frame, a different group of 9 electrodes functions as the first electrode group 2302. FIG. 24 is a schematic diagram showing two measurement frames for such a method. In a first measurement frame 2402, the 9 darkest-shaded electrodes form the first electrode group 2302, the medium-dark grey shaded electrodes form the second electrode group 2304, and the lightest grey electrodes form the third electrode group 2306. After measurement signals have been obtained at the 9 electrodes of the first electrode group 2302 in frame 2402, a new measurement frame 2404 is established in which the 9 darkest-shaded electrodes in frame 2404 form the new first electrode group 2302, the medium-dark grey shaded electrodes in frame 2404 form the new second electrode group 2304, and the lightest grey electrodes in frame 2404 form the new third electrode group 2306. Notably, the electrodes of the first electrode group 2302 in frame 2404 are adjacent to the electrodes of the first electrode group 2302 in frame 2402. Measurement signals are obtained from the 9 electrodes of the first electrode group 2302 in frame 2404, and then the procedure continues in analogous fashion, with each new measurement frame including a different group of 9 electrodes forming the first electrode group 2302, until measurement signals have been obtained from all of the electrodes associated with the sample location.

In the measurement configuration described above in connection with FIGS. 23 and 24, the electrodes of the first group 2302 function as both stimulation electrodes (which deliver electrical stimuli to sample 2202) and measurement electrodes (at which measurement signals corresponding to the sample response to the stimuli are obtained). However, this measurement configuration is only one example of a vertical field measurement configuration. The measurement devices described herein can be used in many other vertical field measurement configurations as well. For example, in some embodiments, the lightest grey shaded electrodes in FIG. 24 function as stimulation electrodes and deliver the AC current signal to sample 2202, the medium-dark grey shaded electrodes function as shielding electrodes and are biased with same AC current signal, and the darkest grey shaded electrodes are biased to a ground potential and function as return and measurement electrodes. In this vertical field measurement configuration, measurement signals are obtained frame-by-frame from the electrodes associated with the sample location in the same manner described above.

As another example, in some embodiments, beyond simple sequential impedance measurements at pairs of adjacent electrodes, the measurement devices described herein can be used to perform concurrent lateral field measurements at multiple sites in a sample. In such measurements, multiple stimulus and return electrodes can be active at the same time among the electrodes associated with a sample location.

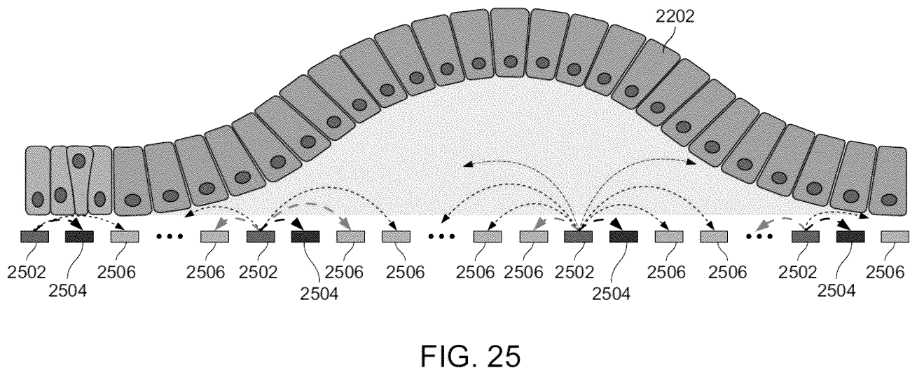

FIG. 25 shows a schematic cross-sectional view of an example of such a measurement. In FIG. 25, multiple stimulation electrodes 2502 apply AC current signals to stimulate sample 2202 at the same time. Electrodes 2504, which are nearest-neighbor electrodes to electrodes 2502, are biased to ground potential and function as measurement electrodes. Two measurement electrodes 2504 are used for every stimulation electrode—a nearest-neighbor in each of two orthogonal directions relative to each stimulation electrode 2502 (only one of which is shown in FIG. 25 due to the cross-sectional view). Measurement signals are detected at electrodes 2504 concurrently. Electrodes 2506 are biased to ground potential and function as shielding electrodes.

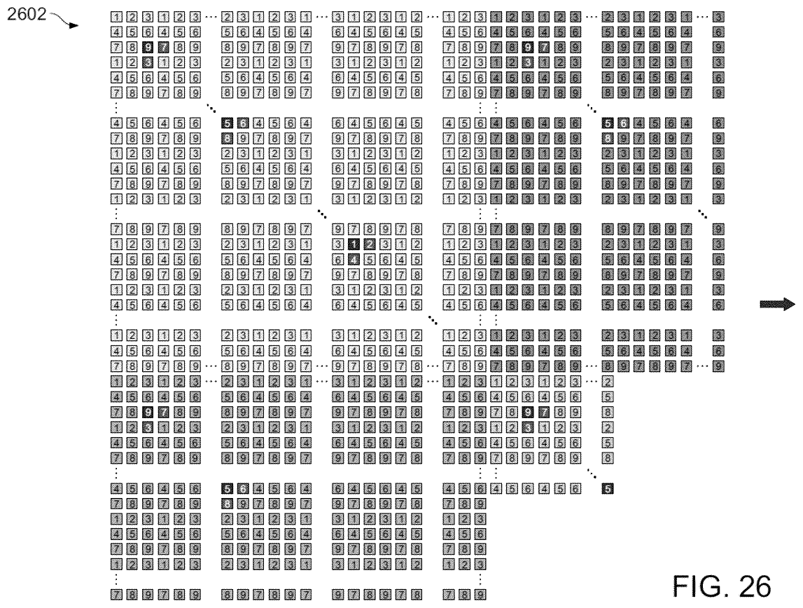

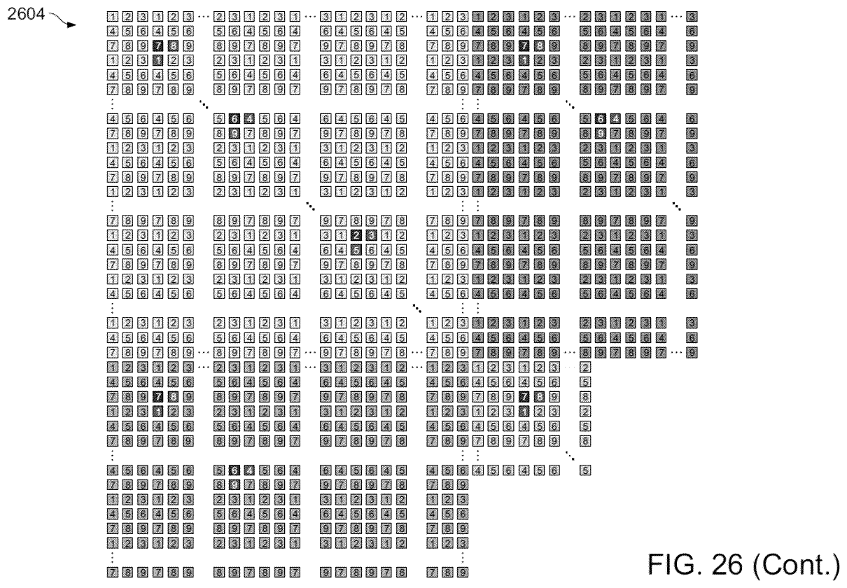

FIG. 26 is a schematic diagram showing multiple measurement frames for the measurement configuration of FIG. 25. In a first measurement frame 2602, stimulation electrodes are shaded darkest grey and labeled with white numbers, measurement electrodes are shaded next-darkest grey and labeled with white numbers, and shielding electrodes are other colors. Multiple stimulation and measurement electrodes are active at the same time so that lateral field measurements at multiple sites can be performed concurrently. After measurement signals at measurement electrodes in the first frame 2602 have been obtained, the electrode configurations are changed for the second measurement frame 2604, and different electrodes are used as the stimulation electrodes and measurement electrodes. It should be noted that each of the stimulation electrodes in second measurement frame 2604 is adjacent to a stimulation electrode in first measurement frame 2602. In other words, in each successive measurement frame, a new group of stimulation and measurement electrodes is configured, until lateral field measurements have been obtained for each of the electrodes associated with the sample location.

Returning to FIG. 25, it is evident from the figure that cell attachment to the surface of substrate 104 affects the electric field return distribution significantly. At low field frequencies, when cells are attached to the surface, the electric field generated by the stimulation electrodes is generally tightly constrained to nearest neighbor electrodes, resulting in a larger current signal detected at the measurement electrodes. Conversely, when cells are less attached to the surface, the electric field generated by the stimulation electrodes is less constrained spatially, and return currents are distributed to a greater extent spatially among both measurement and shielding electrodes. Consequently, detected current signals at the measurement electrodes are generally of smaller magnitude.

However, at high field frequencies, when cells are attached to the surface, the presence of the cells in close proximity to the electrodes blocks the field from nearest neighbor electrodes. With a more constrained current path between neighboring electrodes, the impedance between the electrodes increases, and the measured current at nearest neighbor electrodes decreases. Conversely, when cells are less attached to the surface, a lower impedance path exists between nearest neighbor electrodes (relative to stronger cell attachment at high field frequencies), and the measured current at nearest neighbor electrodes increases.

It is evident from the foregoing that frequency-based lateral field measurements can sensitively probe cell attachment, but the measured signal will depend among other factors on the frequency of the field that is introduced. At very low frequencies, larger current signals will be measured at nearest neighbor electrodes (relative to a stimulation electrode) than at electrodes further away from the stimulation electrode. At very high frequencies, smaller current signals will be measured at nearest neighbor electrodes than at electrodes further away from the stimulation electrode. At frequencies between very low and very high, the measurement signals measured at nearest neighbor and more distant electrodes from the stimulation electrode will reflect an intermediate position between these end points. That is, as the frequency is changed from very low to very high, current signals detected at nearest neighbor electrodes will become relatively smaller (i.e., relative to measured current signals at very low frequency) while current signals detected at nearest neighbor electrodes will become relatively larger (i.e., relative to measured current signals at very low frequency). With suitable calibration and normalization, the cell attachment profile for the sample can be quantitatively determined from the current signals detected at the measurement electrodes.

Analogous effects are observed for the vertical field measurements described above in connection with FIG. 23. At very low frequency, the electric field introduced by a stimulation electrode passes through the tight junction, and the measured signal at a measurement electrode is related to the transepithelial electrical resistance (TEER). At very high frequencies, however, the electric field instead passes through the cell membrane due to its capacitive nature, and the measured signal at a measurement electrode is related to the height of the cell (or cell layer). At an intermediate frequency between very low and very high, the detected measurement signal at a measurement electrode includes contributions from both effects, and therefore contains partial information about both the TEER and cell height. With suitable calibration (and, in some embodiments, by detecting measurement signals in response to stimulation at multiple frequencies), both the TEER and cell height information can be obtained.

More generally, by stimulating samples with different frequencies, a variety of different sample responses can be induced. The sample responses contain “orthogonal” components in the sense that a sample's response to a particular stimulation can reflect the stimulation's probing of different biological structures and/or response mechanisms. The detected measurement signal may therefore contain contributions from more than one “orthogonal” component, depending upon the frequency of the stimulus provided to the sample.

As described previously, the measurement devices described herein allow stimulus signals that include multiple different frequency components to be provided to a sample. Detected measurement signals in response to such multi-frequency stimuli can include convolved contributions from different types of phenomena that are probed, all of which can be obtained from a single, multi-frequency measurement.

Orthogonal information—that is, information relating to different, nominally unrelated biological structures and/or responses—can be extracted from detected measurement signals using a variety of methods. As will be described in more detail subsequently, principal component analysis (PCA) can be used to deconvolve this orthogonal information. Other methods such as, but not limited to, linear discriminant analysis, K-means clustering, projection pursuit analysis, and analysis via support vector machines, neural networks, and other clustering algorithms, can also be used to extract orthogonal information from complex detected measurement signals.

Certain types of orthogonal information that is extracted is attributable to specific biological structures, attributes, or responses. For example, when PCA analysis is used to analyze detected measurement signals, certain principal components will be strongly correlated with particular structures, attributes, or responses of the sample to a stimulus (e.g., TEER, cell height, cell attachment). Other types of orthogonal information—that is, other principal components which PCA analysis is used—will not necessarily be strongly correlated with specific sample structures, attributes, or responses to stimuli. Nonetheless, these types of information also reflect—in some manner—attributes of, or changes in, the sample that are induced by the applied stimulus.

The measurement devices and methods described herein can be used to obtain multi-dimensional, complex data sets that include both types of orthogonal information: that is, types of orthogonal information that are directly and specifically biologically correlated, and types of orthogonal information that are indirectly and non-specifically biologically correlated. Both types of orthogonal information can be used in live cell profiling for a diverse array of applications involving a variety of chemical and genetic perturbations of the cells. Examples of such applications will be described in greater detail subsequently.

Returning to step 2106 of flow chart 2100, another aspect of determining the measurement configuration involves selecting the type of measurement signal that will be measured. Because electrical signals are routed from electrodes 902 through their connected pixel circuits to peripheral circuits 908 for detection, detection of different types of measurement signals can be performed by appropriately configuring peripheral circuits 908 connected to electrodes 902.

As described above, peripheral circuits 908 can be configured for operation in a variety of different modes. For example, in some embodiments, to detect current signals at electrodes 902, peripheral circuits 908 can be configured for operation in the first configuration shown in FIG. 16A, in which amplifier 1538 functions as a trans-impedance amplifier to amplify current signals received from electrodes 902.

As another example, in certain embodiments, to detect voltage signals at electrodes 902, peripheral circuits 908 can be configured for operation in the third configuration shown in FIG. 16C, in which amplifier 1538 functions as a capacitive gain voltage amplifier to amplify voltage signals received from electrodes 902.

An additional aspect of determining the measurement configuration involves determining the spatial measurement resolution. For certain measurements, the spatial measurement resolution is effectively selected based on the nature of the signal that is measured. For example, when nearest-neighbor detection of current signals or voltage signals are performed, the spatial measurement resolution is established by the nearest-neighbor distances between electrodes at the sample location.

Some measurements, however, offer flexibility in the selection of the spatial measurement resolution. Typically, this flexibility manifests in the selection of measurement electrodes at which measurement signals are obtained. In the most straightforward measurement configurations, groups of immediately adjacent electrodes function as measurement electrodes, with the spatial resolution determined based on the distances between the immediately adjacent electrodes, as shown for example in FIG. 24.

In certain embodiments, however the spatial measurement resolution can be selected based on factors such as the nature of the sample, the desired acquisition time for obtaining measurement signals across the entire measurement device, and the power consumption of the measurement device. Lower spatial resolution measurements can generally be performed more rapidly and consume less power. Consequently, such measurements can be useful, for example, for identifying samples of interest, and then later obtaining measurement signals from the samples of interest at higher spatial resolution. Measurement signals can also be obtained at lower spatial resolution where the nature of the measurement is such that higher resolution affords little to no additional useful information about the sample.

Measurements can be performed at a desired spatial measurement resolution by selecting appropriate electrodes at each sample location to function as measurement electrodes. An example of selecting from among multiple different spatial resolutions by designating different electrodes as measurement electrodes is described above in connection with FIGS. 12A-D. In general, the same or analogous methods can be used at each sample location to obtain measurement signals at a desired spatial resolution.

After the measurement configuration has been determined in step 2106, the integrated circuits of measurement device 100 are configured for measurement in step 2108. In general, configuration of integrated circuits 102 for measurement includes configuration of pixel circuits 1054 connected to individual electrodes 902 to control the mode of operation of the electrodes, i.e., the current and/or voltage signals applied by the electrodes to the sample, and the measurement signals obtained at the electrodes in response to stimulation of the sample, configuration of peripheral circuits 908 to which electrodes 902 are connected through their pixel circuits 1054 to control the nature of the measurement signals detected at the electrodes, and configuration of DAC 1700 to control the nature of current and voltage signals generated in measurement device 100 and supplied to the electrodes.

In some embodiments, different electrodes are used as stimulation electrodes and measurement electrodes and therefore the stimulation and measurement electrodes can be configured for operation at the same time, e.g., prior to obtaining any measurement signals. In certain embodiments, however, certain electrodes function as both stimulation and measurement electrodes. Accordingly, the pixel circuits of such electrodes can first be configured to deliver stimulation current and/or voltage signals to sample 2022, and then after the current and/or voltage signals have been applied, the pixel circuits of such electrodes can be reconfigured so that the electrodes rout measurement signals to connected peripheral circuits 908 for detection. In this respect, some of the configuration operations that are nominally performed as part of step 2108 can be interleaved with certain operations that are performed as part of step 2110.

Following configuration of the integrated circuits, measurement signals are obtained in step 1110. To obtain measurement signals, stimulation electrodes deliver current and/or voltage signals to sample 2202 and measurement signals reflecting the response of sample 2202 to the stimulation signals are obtained at measurement electrodes. In measurement configurations where certain electrodes are used as both stimulation and measurement electrodes, these electrodes can be re-configured after providing stimulation signals to the sample as discussed in connection with step 2108.

As described previously, peripheral circuits 908 are selectively connected to electrodes to detect measurement signals that are obtained at the electrodes. The detected measurement signals are then routed from peripheral circuits 908 by multiplexer 1320 to one or more ADCs 904, where they are converted into digital signals that include measurement information derived from the detected measurement signals. The digital signals are then transmitted, in sequential order from multiple ADCs 904, to an external host (e.g., host controller 706) via a data output interface that includes multiple data output lines.

After the measurement information for each of the samples 2202 has been transmitted by measurement device 100, the procedure shown in FIG. 21 reaches a decision step 2112. If all measurements for the samples have been performed, then the procedure terminates at step 2114. However, if further measurements of the sample are to be performed, then the procedure returns to step 2106, at which a new measurement configuration (for a different type of sample measurement) is determined. The procedure shown in FIG. 21 can be implemented in multiple cycles. Different types of measurements can be performed on the samples in each cycle. Alternatively, or in addition, certain cycles may repeat previously performed measurements. For example, measurements can be repeated on the same samples in a time series to monitor changes to the samples over an extended period of time. Measurements can also be repeated for purposes such as, but not limited to, verification of measurement information, signal averaging, and/or calibration.

The procedure described in connection with flow chart 2100 is merely an example of a method for obtaining measurement information for multiple samples using the measurement devices described herein. It should be understood that measurement methods can include additional steps not shown in flow chart 2100, and can include various modifications to the method steps described in connection with flow chart 2100. In addition, the steps of flow chart 2100 can each be performed in manner different from the description of the steps contained herein, as measurement device 100 can readily be adapted to a wide variety of different configurations and measurement procedures.

### EXAMPLES

High-throughput screening is the dominant paradigm for profiling compounds based on biological activity, toxicity, and mechanism of action (MOA). One of the most informative screening tools is high-content imaging with feature extraction to create high-dimensional profiles (e.g., Cell Painting). However, the technique only produces an end-point image of fluorescently labeled fixed cells, missing important characteristics of live cells and tissues. For example, barrier and water transport properties of epithelia are important for cancer, fibrosis, inflammation, and cystic diseases, yet they cannot be assessed easily using high-content imaging.

Impedance techniques can overcome the shortcomings of imaging, providing live-cell morphology information in real time, throughout the full experiment time-course. They bring the additional advantages of being non-invasive and label-free, enabling experiments without fluorescent probes or cell line engineering. Transepithelial electrical resistance (TEER) assays and other commercial devices (e.g., xCelligence RTCA from Agilent Technologies, Inc., ECIS from Applied Biophysics, Inc.) measure the impedance of cell membranes to examine tissue barrier and kinetics (cell growth/death). Nonetheless, these devices use large electrode pairs for measurement resulting in limitations in both accuracy and the number/type of parameter readouts.

The following examples use the measurement devices described herein to provide a high-throughput, high-resolution impedance platform for drug discovery applications and to perform high-dimensional MOA profiling and phenotypic hit/lead generation. The measurement devices included 96 integrated circuits on a substrate, each including 4096 electrodes that provided a spatial resolution of 25 μm for high-dimensional measurements. More than 20 parameters of the samples were measured using different stimulation frequencies, field geometries, and spatial features. The parameters were measured every 5-15 minutes in real-time to obtain a full kinetic view of live-cell growth and compound responses, providing functional insights beyond end-point imaging.

The sensitivity and diversity of the measurements created a high-dimensional electrical representation of cell state over time, involving the characterization of eleven different cell types ranging from primary epithelial, to cancer epithelial, to suspension, each with unique morphology and growth dynamics. Through screens of 341 FDA approved compounds, the measurement devices and systems demonstrated real-time, label-free profiling of compound MOA on live-cells. In addition, the translatability of the unique parameters has applicability to phenotypic high-throughput screening for related drug discovery applications, including epithelial to mesenchymal transition (EMT), transepithelial water transport, and barrier function. Identification of previously unknown compounds that increase tissue barrier suggested the ability of the measurement devices and systems to help identify novel therapeutic approaches in the context of gut barrier diseases.

Integrated circuits were positioned within each well of a 96-well measurement device. Each well had a 140 μL maximum capacity and 120 μL working volume with a bottom diameter of 3.4 mm. A 64×64 array of 4,096 pixels at a 25 μm pitch for each integrated circuit resulted in a 1.6×1.6 mm² total sensing area. Electrochemical images with 25 μm resolution were generated across the full 96-well measurement device in 40 seconds. Parallelized data signal transfer allowed for operation of, and data reception from, 8 measurement devices simultaneously. Real-time, live-cell impedance data was typically acquired at a frame per 5-15 minutes and fed through a cloud-based data processing pipeline for analysis. Analysis included feature extraction, high dimensional clustering, and integration with known compound metadata to identify phenotypes and relate them to cell function.

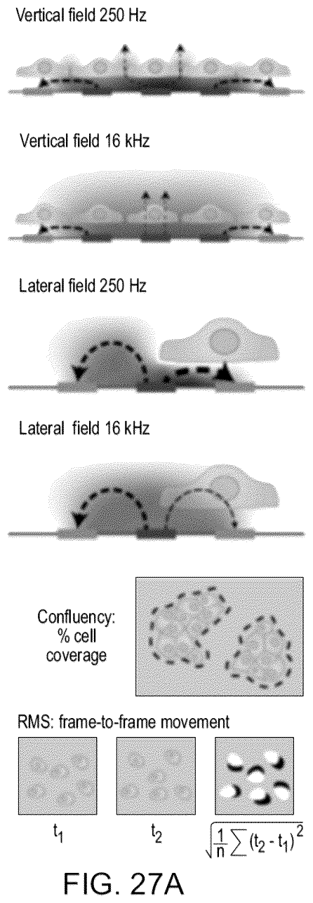

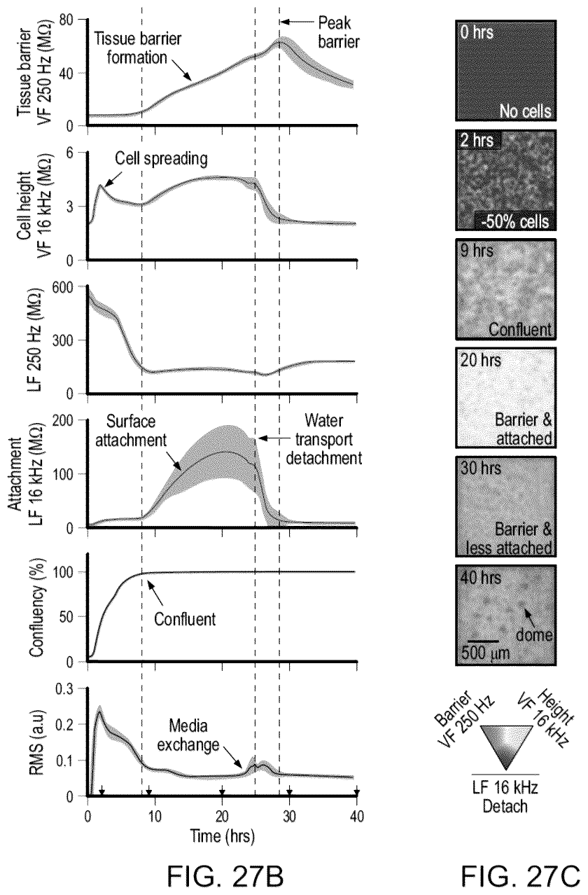

Impedance measurements were performed with two different electric field configurations named vertical field (VF) and lateral field (LF) and at four different frequencies of 250 Hz, 1 kHz, 4 kHz, and 16 kHz, as shown in FIG. 27A. In total, 9 impedance parameter maps were measured per field configuration, including magnitude and phase for the 4 frequencies and a Direct Current (DC, 0 Hz) magnitude. To help explain the measurements and to demonstrate the orthogonality of the parameters, a specific experiment is illustrated in FIGS. 27A-27C using MDCK cells, an epithelial cell line that forms a strong cell barrier and has demonstrated apical to basal water transport resulting in tissue doming or hemicysts. Connections were observed between biological information and measurements at frequency magnitudes, phases, and DC.

To perform field-based impedance measurements across multiple frequencies, a voltage stimulation was applied which is the summation of the four different frequency signals and return currents were measured using a transimpedance amplifier (TIA) configuration with a feedback gain of 18 MΩ. The magnitudes of the AC voltage signals were scaled to create similar output amplitudes that were measured by the TIA configuration (0.2 V/250 Hz, 0.08 V/1 kHz, 0.04 V/4 kHz, and 0.02 V/16 kHz for lateral field, 0.25 V/250 Hz, 0.1 V/1 kHz, 0.04 V/4 kHz, and 0.025 V/16 kHz for vertical field). Six of the 96 wells were scanned at a time taking 2.5 s, resulting in a total scan time across the well plate of 40 s. A fast Fourier transform (FFT) was used to extract the magnitude and phase of each of the four frequencies and the DC component—9 impedance parameter maps for each field configuration. It was observed that different frequencies contained different types of biological information (as shown in FIGS. 27A-D).

A reference impedance map was used to determine the location of cells: a threshold was set above the electrodes' default impedance in solution, and the presence of a cell above an electrode then increased the impedance beyond the threshold for detection. Typically, the VF 4 kHz map provided the best contrast for generating the cell mask. For magnitude, phase, and DC, the median value of electrodes with cells was then calculated. An additional epoxy mask was calculated in a similar manner to remove electrodes which had spillover epoxy from well plate attachment. Before each cell plating, a reference measurement was taken in empty culture media to calculate the epoxy mask.

Transient features such as motility and migration were generated from impedance videos through a root-mean-square (RMS) calculation. The difference between two unmasked image frames was taken (the epoxy mask was still used if applicable), then the RMS calculation was performed across the pixels of the difference map. To normalize for changes in magnitude, the calculated RMS was then divided by the median value of the cell-masked well distribution. Typically, the VF 4 kHz map was used for the RMS signal generation.

To execute and analyze the screens, cells were seeded on five CMOS 96-wellplates, allowed to grow for 24 hours for A549 or 48 hours for Caco-2, compounds were added at 10 μM in 1% DMSO via a half media swap using an OpenTrons liquid handling robot. Effects were measured for 48 hours, with impedance measurements taken every 15 minutes. The many field/frequency and calculated parameters were then normalized to a timepoint one hour before compound addition. The array of normalized parameters at timepoints logarithmically spaced from compound addition to +48 hours were then used for a principal component analysis (PCA) and unbiased clustering using the top 20 PCA dimensions. It was found that the logarithmic time spacing balanced rapid binding effects and longer-term effects increasing/decreasing over the full 48 hours.

All cell lines were obtained from ATCC (Manassas, VA) and maintained in a humidified incubator at 37° C. and 5% CO₂. MCF-7, A549, MDA-MB-231, MDCK, Calu-3 and HCT116 cells were cultured in DMEM supplemented with 10% FBS. Caco-2 cells were cultured in EMEM supplemented with 20% FBS. K-562 cells were cultured in IMDM supplemented with 10% FBS. HT-29 cells were cultured in McCoy's 5A medium supplemented with 10% FBS. hCMEC/D3 cells were cultured in EndoGRO media obtained from MilliporeSigma (St. Louis, MO).

Cell lines were seeded in the wells at various densities. Collagen-1—rat tail was obtained from Corning (Corning, NY), and plates were coated according to manufacturer's instructions. All measurements both pre- and post-compound treatment were performed in an incubator regulating CO₂, humidity, and temperature.

For immunofluorescence imaging, cells were grown on coverslips and then either allowed to grow for 48 hours or treated with relevant compounds for 48 hours. Cells were then fixed in 4% paraformaldehyde, washed, permeabilized with 0.5% Triton-X 100 in PBS, and then subjected to antibody incubation. Antibodies used were anti-E-Cadherin (obtained from Cell Signaling Technology, Danvers, MA, as cat no: 3195T), anti-ZO1 (obtained from Thermo Fisher, Waltham, MA, as cat no:617300) and anti-Occludin (obtained from Thermo Fisher as cat no: 331500). Alexa Fluor 488 and 568 secondary antibodies were used. Coverslips were mounted in Vectashield® antifade mounting medium with DAPI.

Imaging was performed using a Yokogawa Wi spinning disk confocal on an inverted Nikon Ti fluorescence microscope equipped with Hamamatsu ORCA-Fusion BT CMOS camera (6.5 μm² photodiode), Lumencor SOLA fluorescence light source, and Nikon LUN-F XL solid state laser combiner: 405 nm (80 mW), 445 nm (35 mW), 488 nm (80 mW), 514 nm (50 mW), 561 nm (65 mW), and 640 nm (60 mW). For imaging, the widefield modality of the microscope was typically used, and all imaging was done using a 20× objective. Acquisition was performed using consistent exposure across samples to enable intensity quantification. Images were processed using the open source image processing software FIJI.

Quantification of ZO-1 in the cell-membrane and nucleus was performed by manually defining ROIs for the cell membrane and nucleus. Mean integrated intensity was calculated within the ROI. All quantification was performed in raw, unprocessed images.

Compound treatments were performed using a half media exchange. To prepare for compound addition, half the media from each well was removed. Compounds were prepared at 2× concentration in cell culture media, and the 2× compound solution was then added to the wells. Care was taken to keep DMSO concentration below 1%, compound dilutions were performed with a constant DMSO concentration. Compound in media was temperature- and CO₂-equilibrated prior to addition. All compounds including the 341 compound library were obtained from Selleckchem Chemicals (Houston, TX). For the screening analysis, the pathway and target information provided by Selleckchem Chemicals was curated together.

At lower frequencies (250 Hz, 1 kHz), the capacitive nature of the cell membrane's lipid bilayer resulted in a very high impedance—in the VF, this caused the field to pass through intercellular spaces resulting in measurement of the permeability of the tissue or barrier. In the MDCK experiment, an increasing VF 250 Hz barrier was observed after confluency was reached reflecting the process of tight junction formation and reached a peak after the onset of water transport. At higher frequencies (4 kHz, 16 kHz), the fields were sensitive to membranes in closer proximity to the electrodes. For the VF configuration, high frequency signals reflected cell size: the flatter the cell, the more cell membranes were near the electrode increasing the impedance. To this end, a spike in the VF at 16 kHz was observed in the experiment shortly after plating attributed to the suspended cells falling to the surface and spreading.

In the high frequency LF, cell substrate attachment was measured with high sensitivity: the closer the cells were to the electrode, the higher the LF 16 kHz impedance. In the MDCK experiment, the cells reached confluence and attached strongly to the surface followed by a rapid detachment with the onset of water transport. The water transport process can be accelerated by media changes, as shown in FIG. 27B, and was also affected by plating density. The spatial resolution of the measurements enabled detection of doming as shown in FIG. 27C, a phenomenon in which transepithelial water transport leads to areas of increased water pressure under the cell sheet. This causes the cell sheet to lift off in dome-shaped structures. In our measurements, domes were observed as circular decreases in attachment signals, represented in the bottom image of FIG. 27C as purple dots of −150 μm diameter.

Beyond fields and frequencies, confluence can be used to parameterize cell growth/death and was calculated as the percentage area covered by cells determined using a threshold as shown in FIG. 27A. Cell locations were also used to mask impedance maps to account for differences in confluency and to omit data from non-covered electrodes, improving accuracy beyond traditional aggregate well techniques. The root mean square (RMS) of the difference from one measurement frame to another was used for parameterizing transient features such as motility and migration.

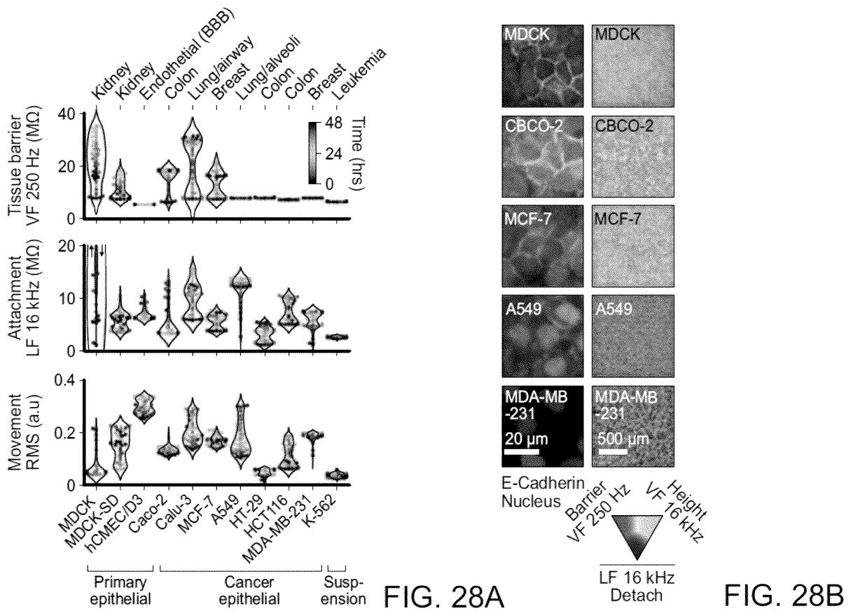

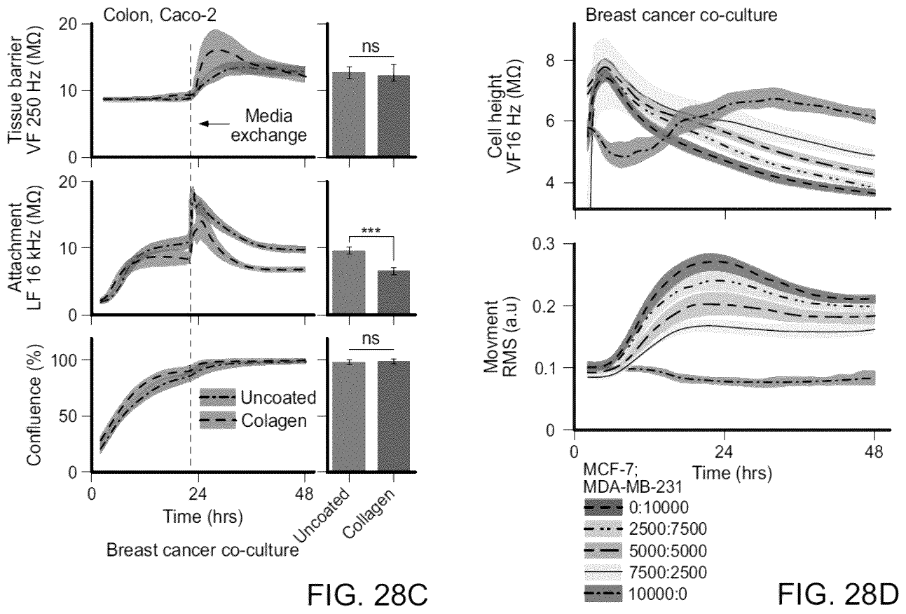

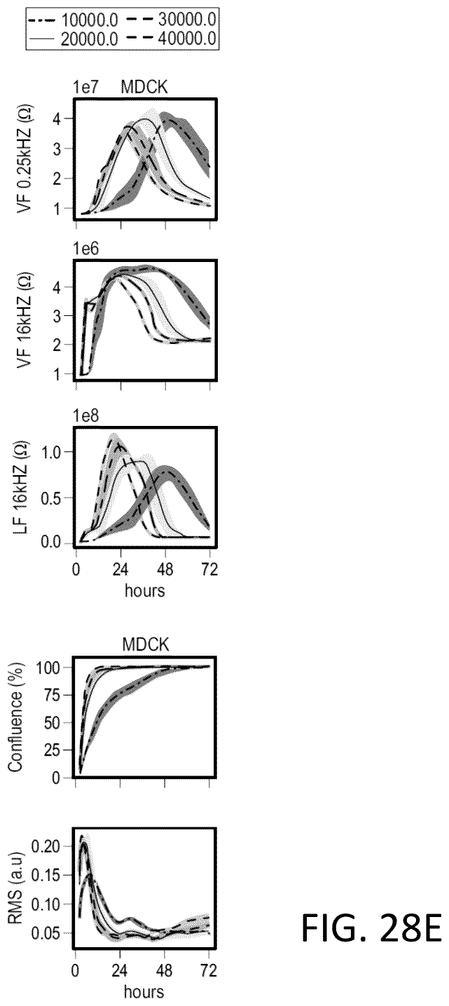

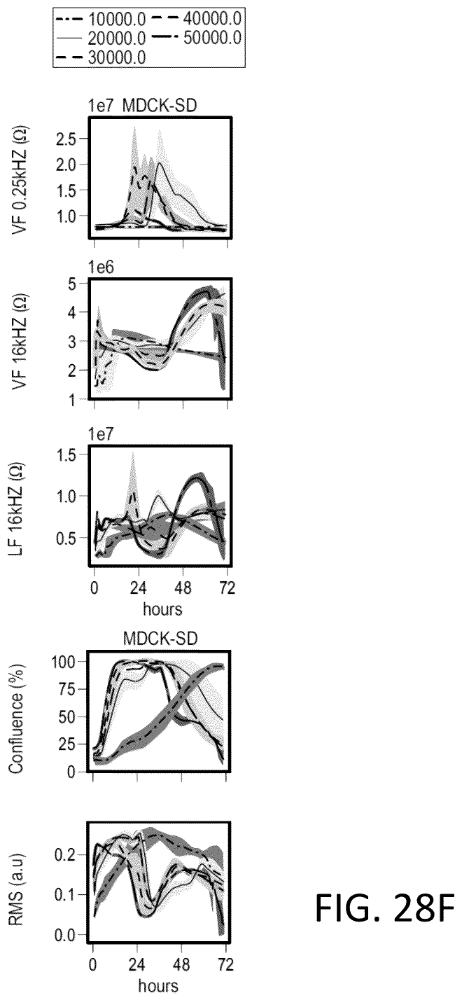

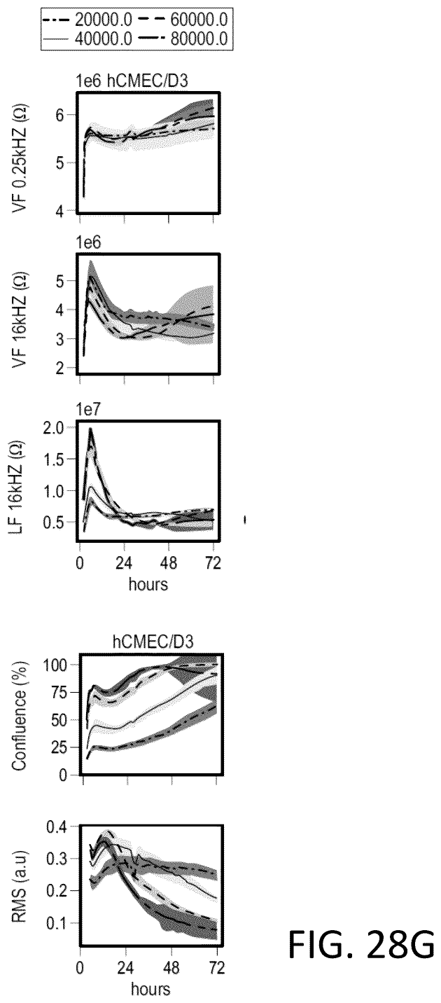

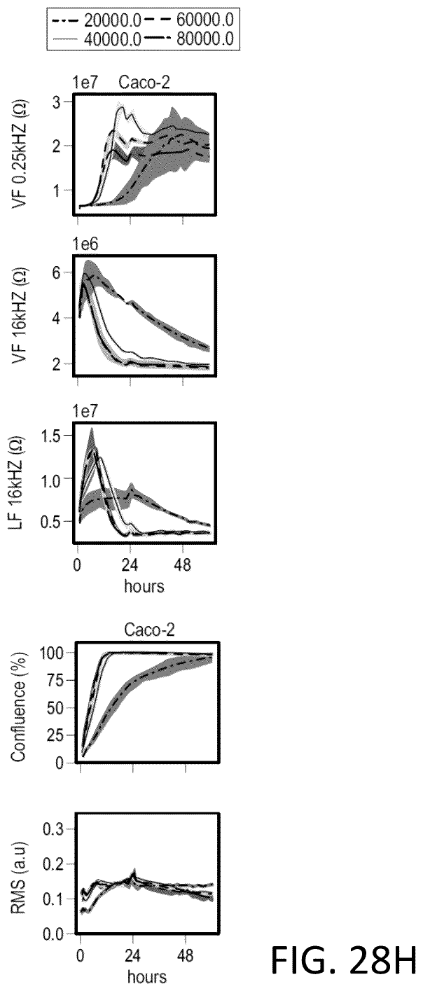

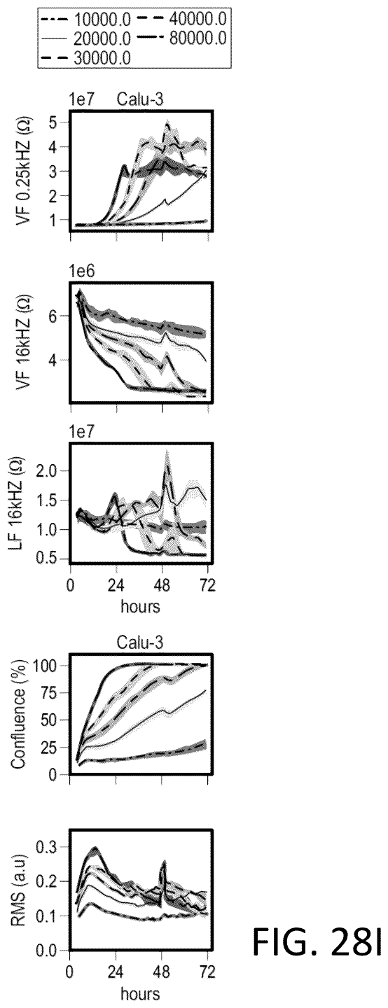

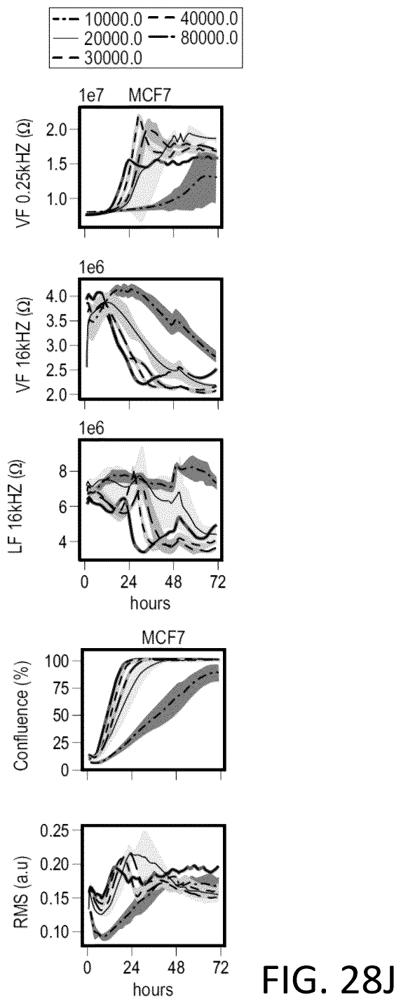

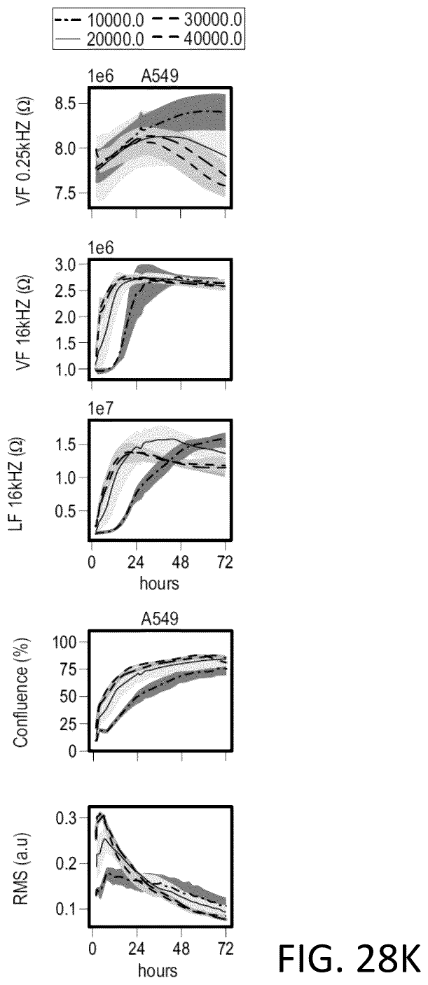

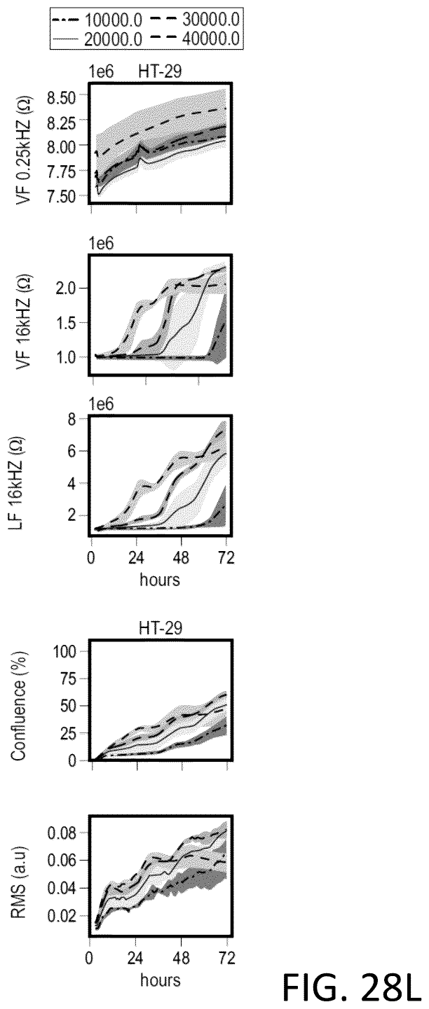

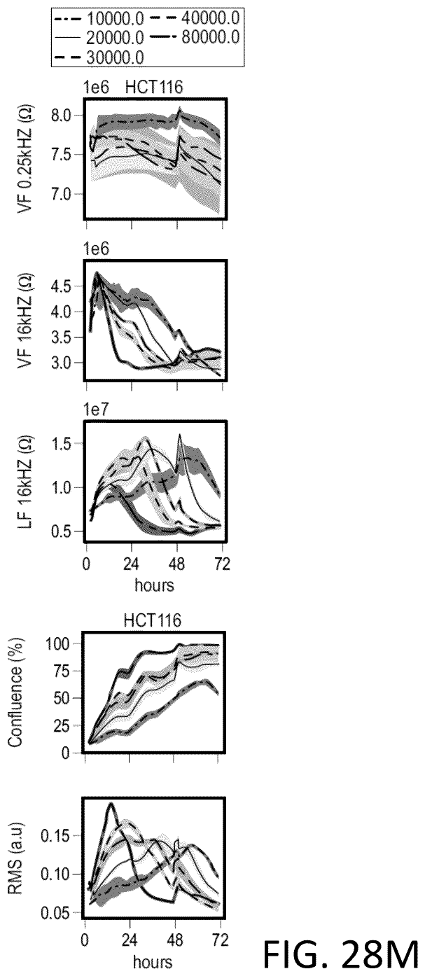

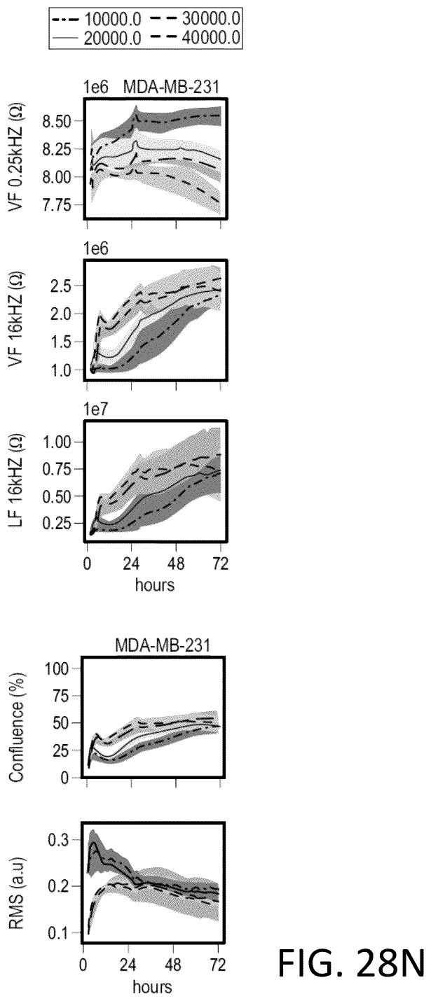

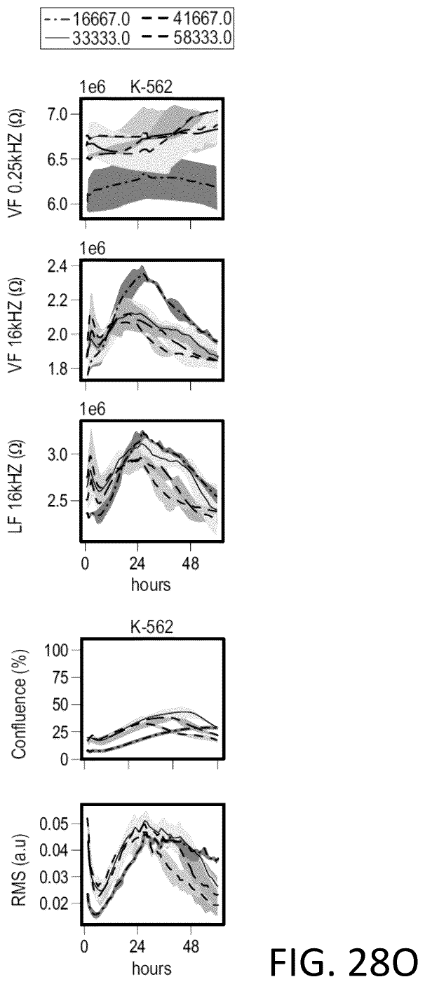

To test the sensitivity of the measurement devices, a wide range of well characterized cell types were measured, spanning from primary epithelial cells to cancer epithelial cells, to suspension cells, as shown in FIG. 28A. The cell types originated from the kidney, brain endothelial/blood brain barrier (BBB), colon, lung, breast, and bone marrow/leukemia, and were of human origin, except the MDCK cells which were canine origin. The impedance techniques measured characteristics of all cell types tested. FIGS. 28E-1 through 28E-6 include a series of plots showing results from different impedance measurements performed for the different cell types at various cell plating densities.

The epithelial cells exhibited a strong attachment to the substrate, and many had tight cell-cell junctions that creates a high barrier as shown in FIG. 28A. Beyond MDCK, these cell types included Caco-2 (colon cancer cells that are used as a model of intestinal barrier and inflammation), Calu-3 (lung adenocarcinoma cells used as a model of bronchial barrier), and MCF-7 (luminal A type breast cancer cells exhibiting differentiated mammary epithelium properties). In contrast, a variety of cell types from similar human tissue origins did not create barriers. These cell types included HT-29 and HCT116 (two types of colon cancer cells), A549 (lung adenocarcinoma cells derived from alveoli), and MDA-MB-231 (triple negative breast cancer cells that have undergone EMT). To complement the cancer cells, hCMEC/D3 (brain endothelial cells used for modeling BBB function) exhibited the highest motility of any cell type. Finally, K-562 (suspension cells from CML patients) were effectively measured and the presence of live cells above the electrodes could be readily identified. As expected, suspension cells had the lowest cell attachment signal of all the tested cell lines.

Immunofluorescence imaging was performed for representative cell types to detect expression and localization of E-cadherin, a functional component of adherens junctions expressed in normal epithelia, and compared the results to impedance images as shown in FIG. 28B. Loss of E-cadherin can cause dedifferentiation and invasiveness in human carcinomas and is observed in cancer cells that have undergone EMT. It was observed that cell types with high-levels of membrane E-cadherin and a regular epithelial morphology (MDCK, Caco-2, and MCF-7) had a correspondingly high barrier signal as shown in the left panel of FIG. 28B. In contrast, cells with low/diffused (A549) or undetectable levels (MDA-MB-231) of E-cadherin did not exhibit a barrier signal as shown in the left panel of FIG. 28B. Different impedance image textures were also observed for the no-barrier cell types: the A549 cells optically had an epithelial morphology and exhibited a smoother impedance texture than the MDA-MB-231 cells which have a mesenchymal morphology and have a more dynamic and rougher texture, as shown in the right panel of FIG. 28B.

Within the body, epithelial cells grow within extracellular matrix (ECM)—integrin signaling through the ECM is an important regulator of epithelial cell polarity and morphogenesis. To this end, the compatibility of coatings was tested by plating Caco-2 cells in wells with or without a collagen type I coating. Results from this testing are shown in FIG. 28C. Cells grown on collagen were successfully measured and had a lower attachment signal reflecting the increased distance of the cells from the electrode (e.g., the LF measurement schematic of FIG. 27A), highlighting the sensitivity of the measurement technique. To further test the limits of sensitivity, an experiment was performed to study breast cancer cells exhibiting distinct phenotypes, co-cultured in different ratios, as shown in FIG. 28D. As observed in FIGS. 28A-C, MCF-7 showed an epithelial morphology and expression of E-cadherin while MDA-MB-231 showed a mesenchymal phenotype with no expression of E-Cadherin. Graded responses corresponding to the ratio of the two breast cancer cell types were observed in the cell size parameter (VF 16 kHz) and movement (RMS) across the mixtures, allowing quantification of the overall epithelial versus mesenchymal phenotype of the mixed populations. Measured and calculated parameter values for different cell types are shown in the graphs of FIGS. 28E-28O.

Compound screening was then performed to help reveal the range of functional phenotypes that can be observed using the measurement devices. Two approaches were taken. In the first, three cell types were chosen, representing a range of characteristics: MDCK, A549, and MDA-MB-231, and a common set of compounds was applied that target various cellular processes. The compounds chosen (Cytochalasin D, Vinblastine Sulfate, Paclitaxel, Alisertib, Bosutinib, Anisomycin, Dexamethasone, Getfitinib, Decitabine, Cyclophosphamide Monohydrate, and GSK 269962A) target various cellular processes including cell division, DNA replication, inflammation and various other signaling pathways. The effect of the compounds on measurements that link to a specific biological parameter was studied. Many diverse changes were observed and matched known effects of the compounds—increases/decreases in attachment, barrier, cell size, motility, and confluency—with differences in response across cell lines.

FIGS. 29D-1 through 29D-1E include a series of graphs showing impedance measurements for MDCK cells following application of each of the compounds above. FIGS. 29E-1 through 29E-10 include a series of graphs showing similar measurements for A549 cells, and FIGS. 29F-1 through 29F-10 include a series of graphs showing similar measurements for MDA-MB-231 cells.

Effects on motility were observed in MDA-MB-231 cells with two different compound treatments: Dexamethasone, an anti-inflammatory drug, and Cytochalasin D, an actin polymerization inhibitor. MDA-MB-231 is established from a metastatic mammary adenocarcinoma and is highly aggressive and invasive. Upon Dexamethasone treatment, an increase in attachment and a decrease in motility (RMS) was observed. Dexamethasone has been described to reverse EMT in MDA-MB-231 cells and reduces their migratory potential corroborating the observed results.

A decrease in motility/RMS was also observed upon Cytochalasin D treatment, but in contrast to Dexamethasone, attachment was decreased and there were significant changes to cell size—an immediate size decrease for ≥0.2 μM with a subsequent increase for ≥1 μM. Cytochalasin D inhibits actin polymerization, which has been reported to prevent cell motility and alter cell shape and size, supporting the observed results. This further demonstrates that the parameters measured using the measurement devices described herein are independent, orthogonal, and interrogate a wide range of cellular features.

Dexamethasone has also been described to increase barrier in A549 cells. The measurements shown in FIGS. 29E-1 through 29E-10 illustrate a similar effect of Dexamethasone that works by increasing the tissue barrier as well as the cell-surface attachment of the cells. Interestingly, in MDCK cells, dexamethasone does not seem to affect any of the morphological parameters as shown in FIGS. 29D-1 through 29D-10. MDCK is a non-cancer cell line with high barrier, low motility, and low levels of inflammation, which might explain the lack of Dexamethasone effect.

In addition to identifying varied effects for compounds, the temporal resolution of the data permitted differentiation between mechanisms of action of drugs with similar outcomes. Paclitaxel and Vinblastine are both microtubule inhibitors, with very similar effects on cell death in A549 cells. As shown in FIGS. 29E-1 through 29E-10, at 48 hours post-treatment, both compounds show similar levels of cell death, reflected in the confluency measurement. However, the cell morphology responses between the two drugs reveal distinct effects. The two compounds differ in their mechanism as Paclitaxel is a tubulin polymerization inhibitor, while Vinblastine is a tubulin depolymerization inhibitor. Thus, they have very different effects on cell morphology. As is evident from the foregoing, the temporal data can thus help differentiate between mechanisms of action.

Of particular interest was an observation that compound treatments modulating water transport properties in MDCK correlated to in vivo results for autosomal dominant polycystic kidney disease (ADPKD). In humans, ADPKD is thought to be caused by a combination of overgrowth of cells and a switch from an absorptive to secretive epithelium, both leading to fluid accumulation in cysts which increases kidney size while decreasing kidney function.

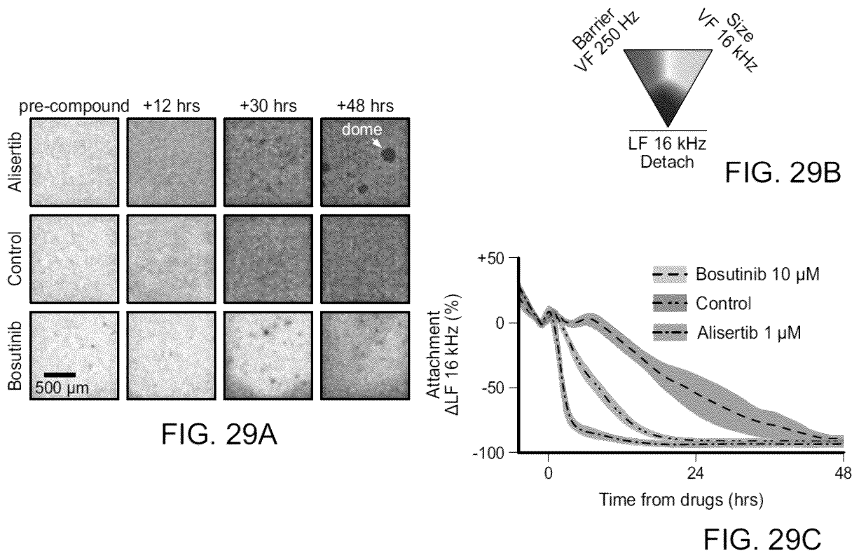

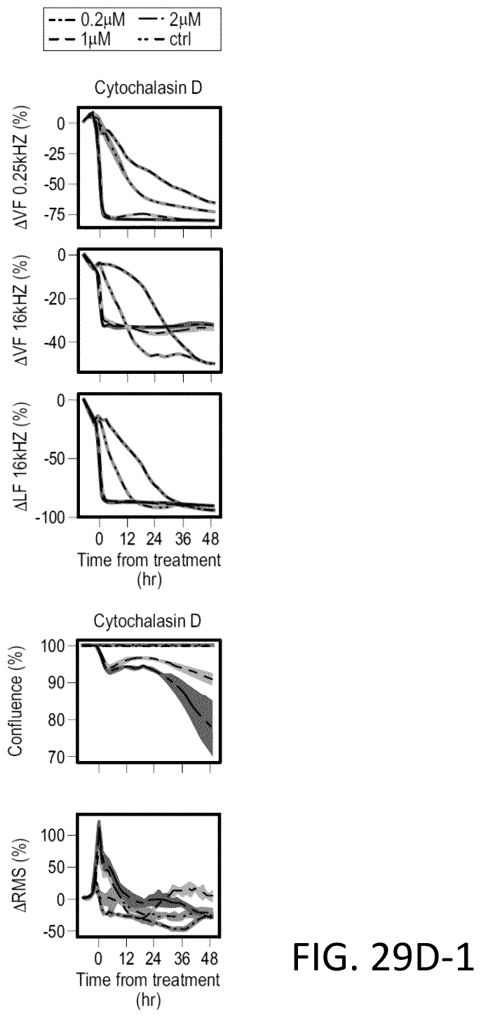

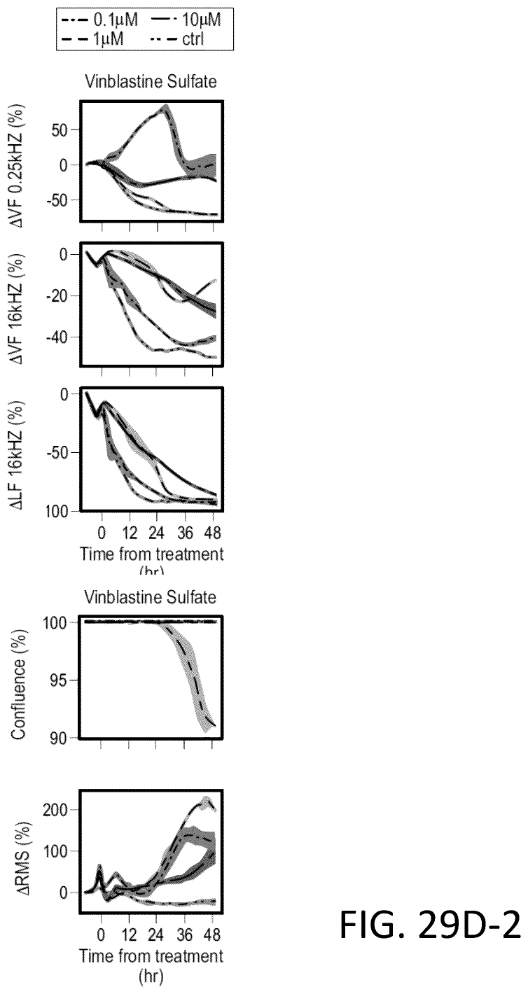

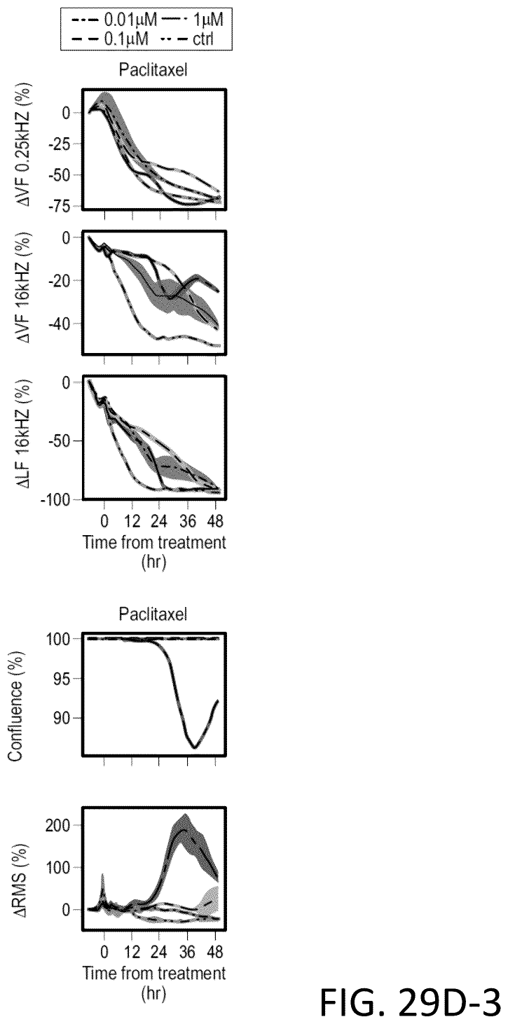

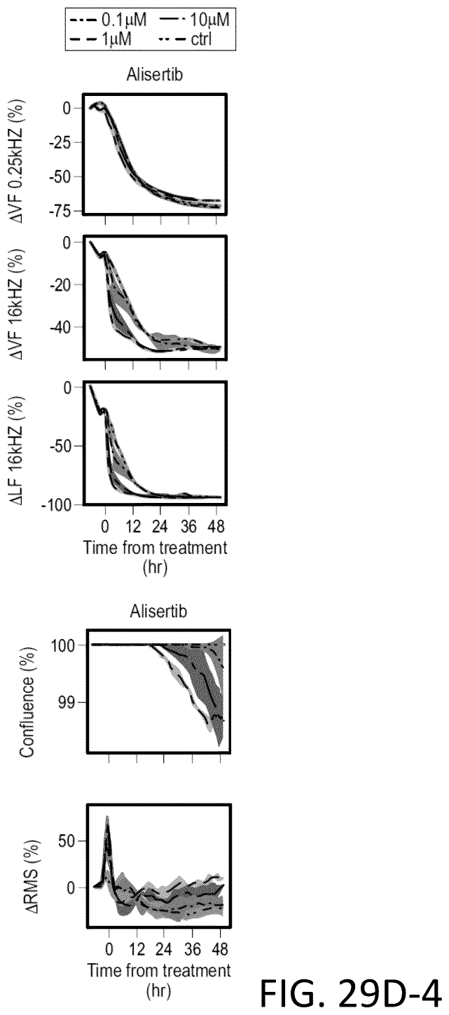

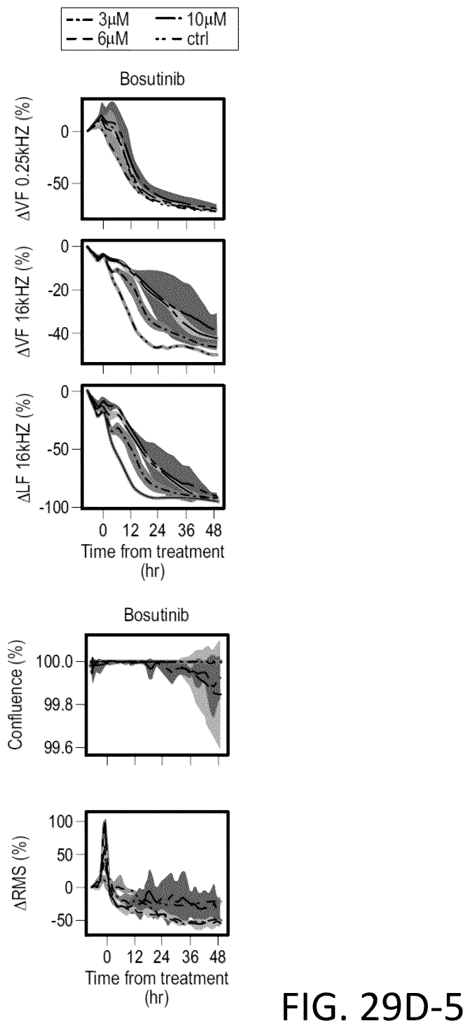

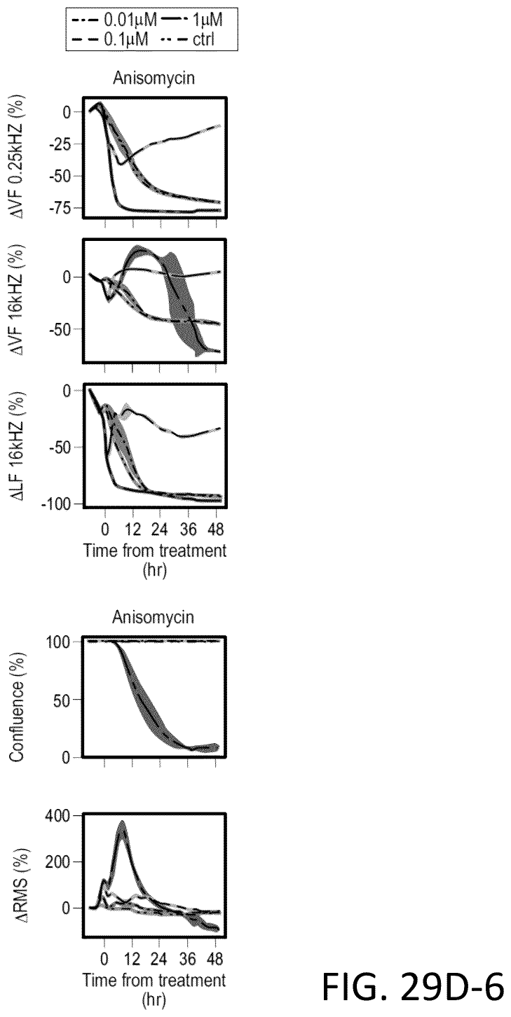

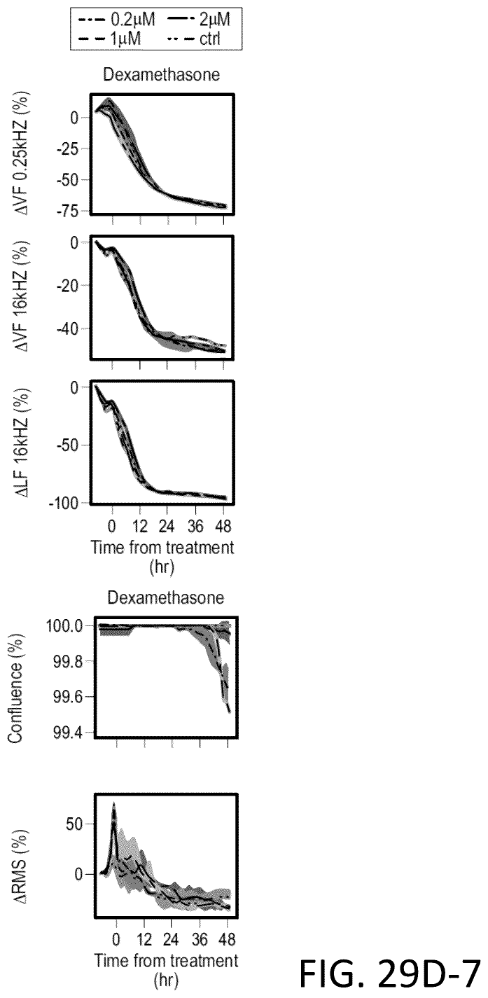

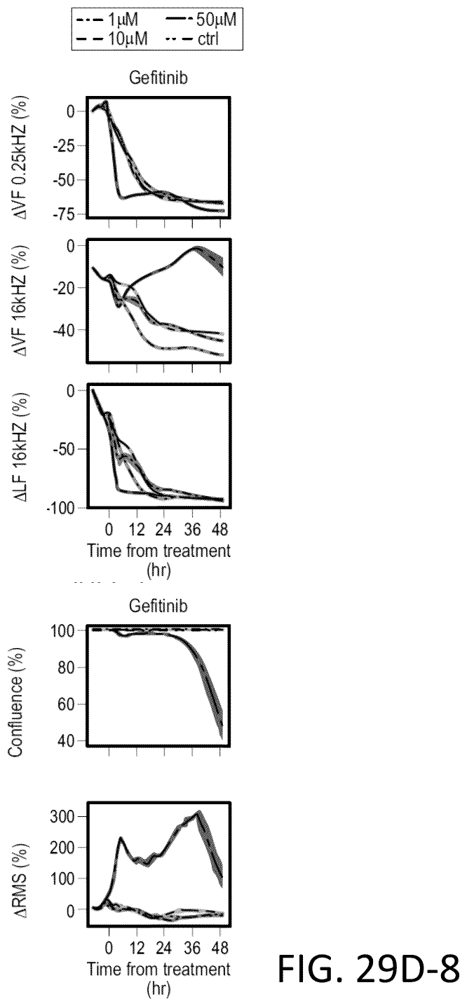

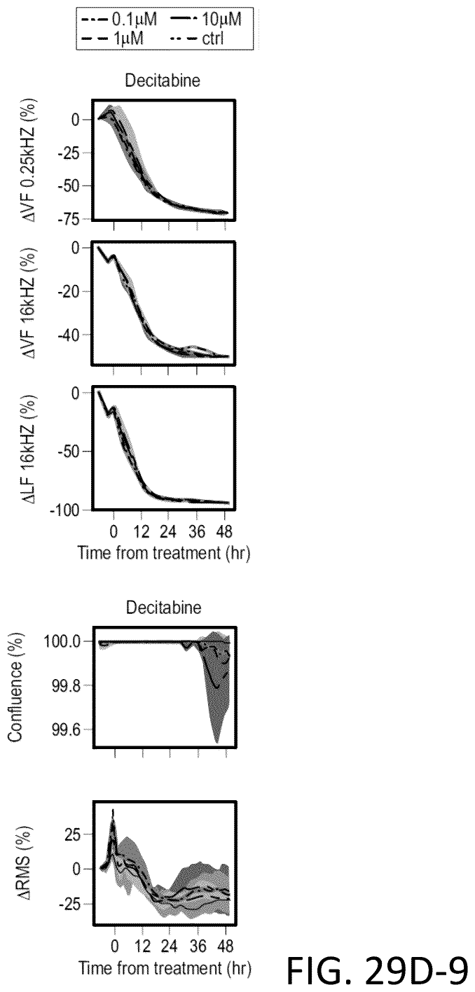

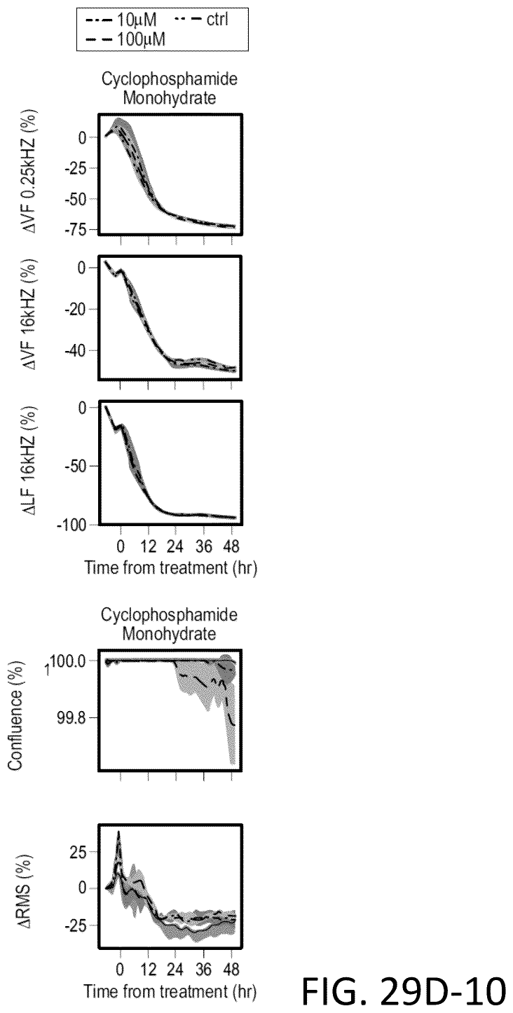

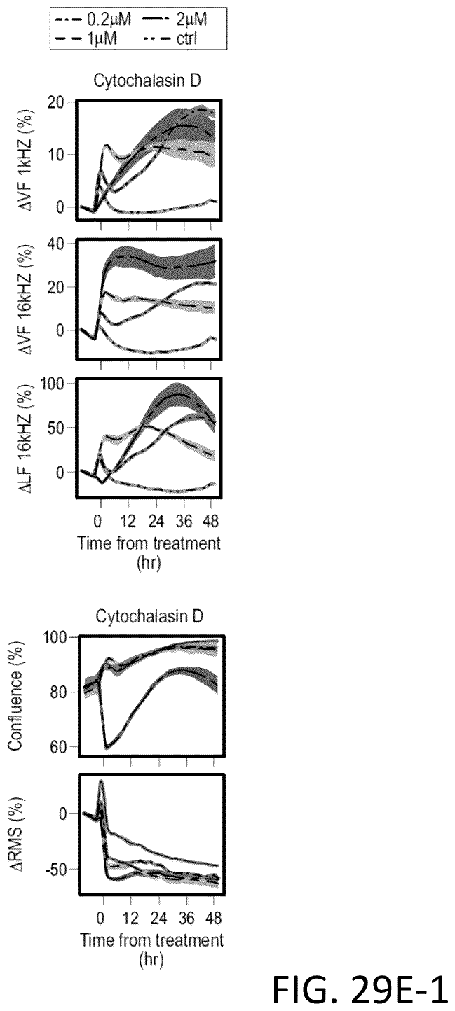

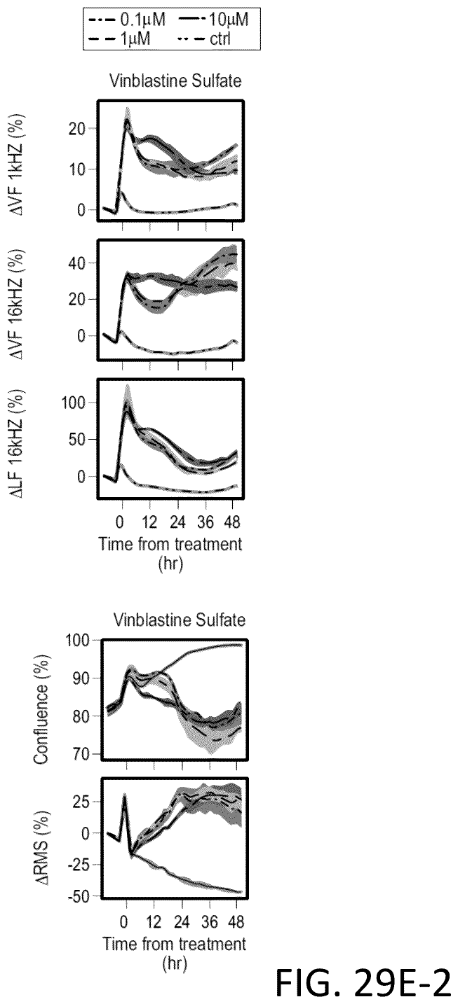

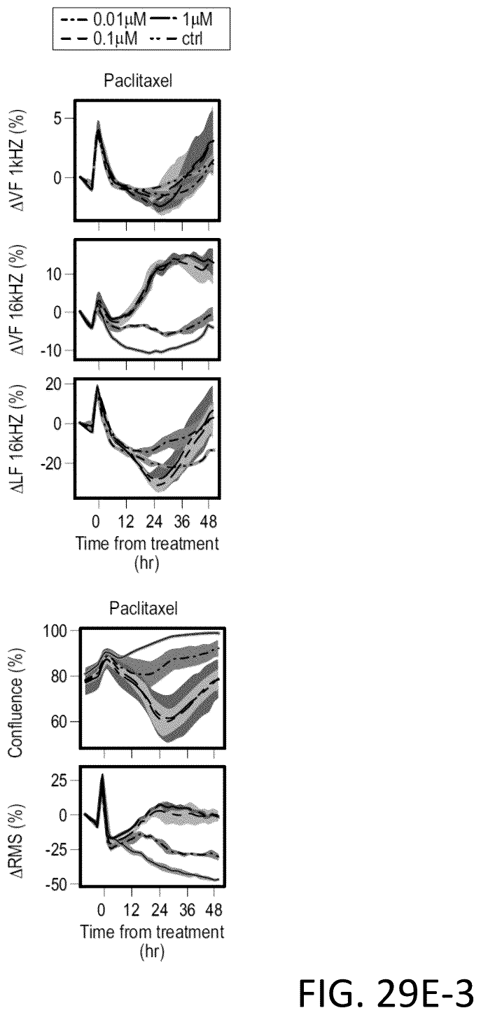

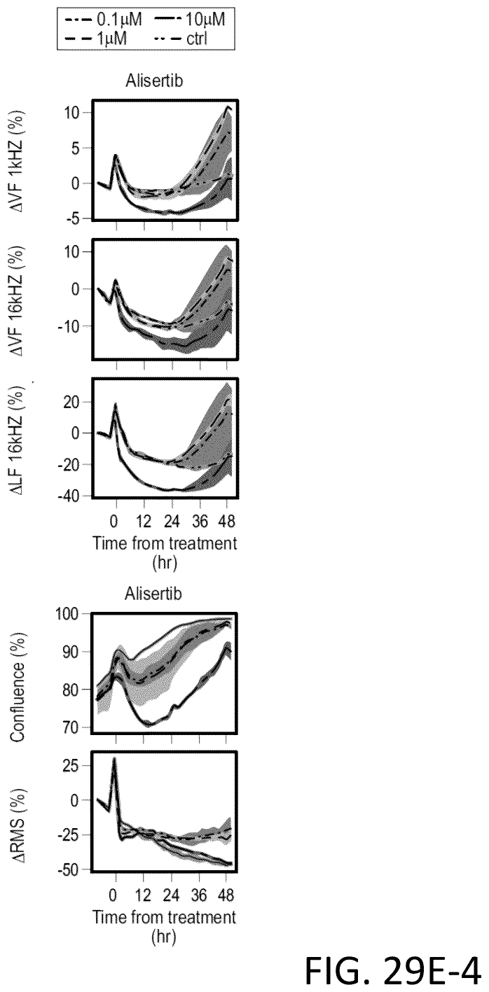

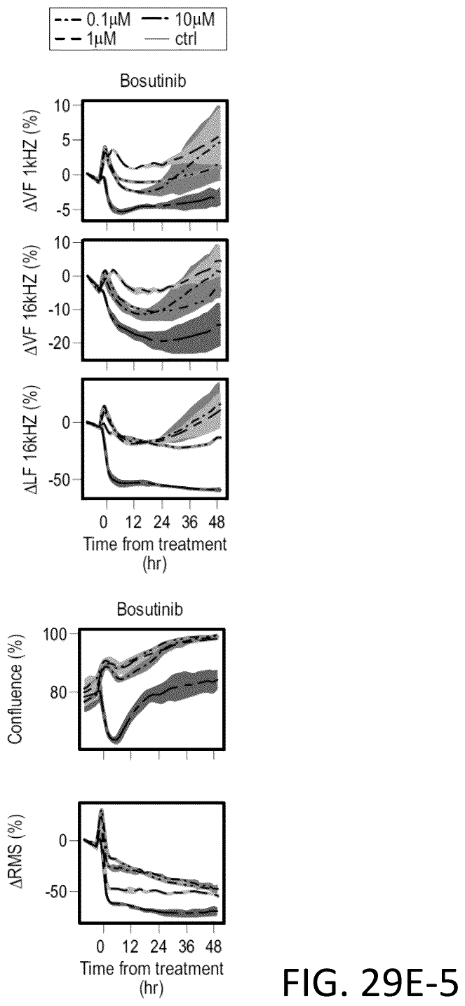

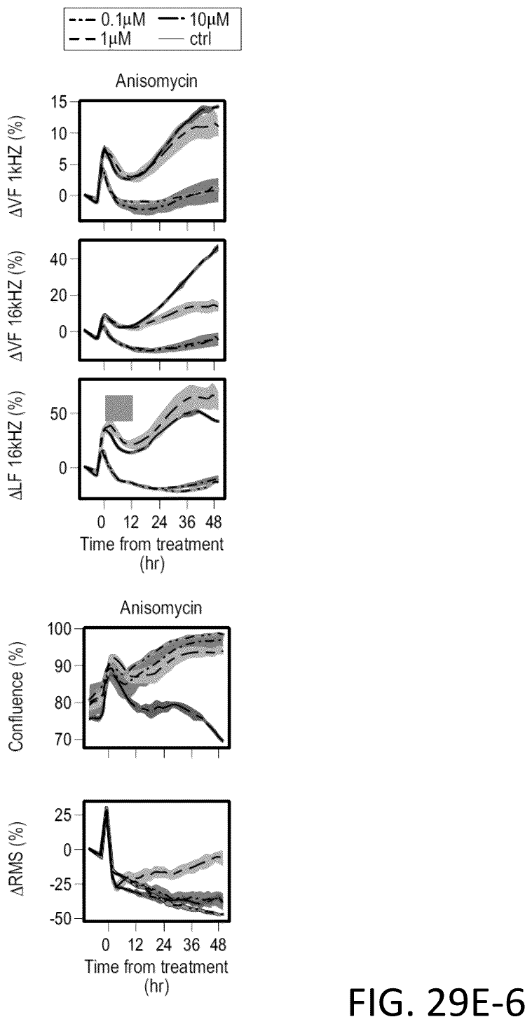

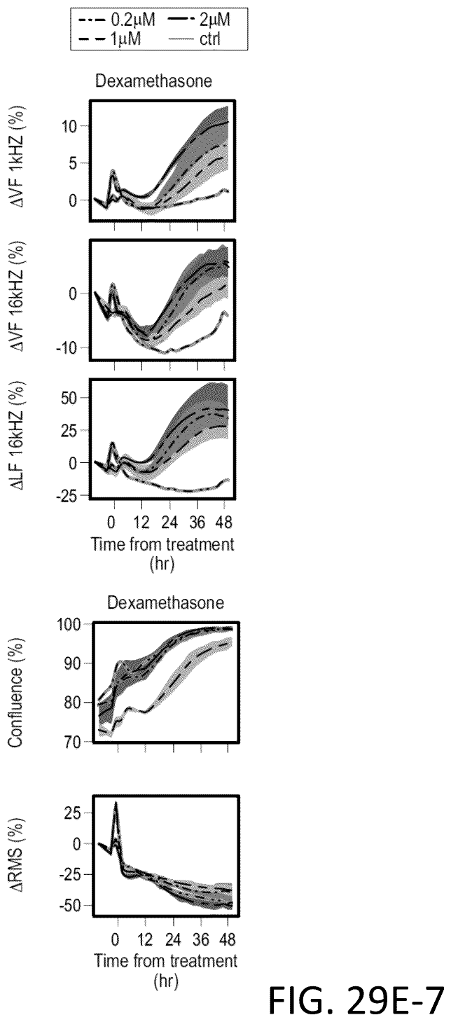

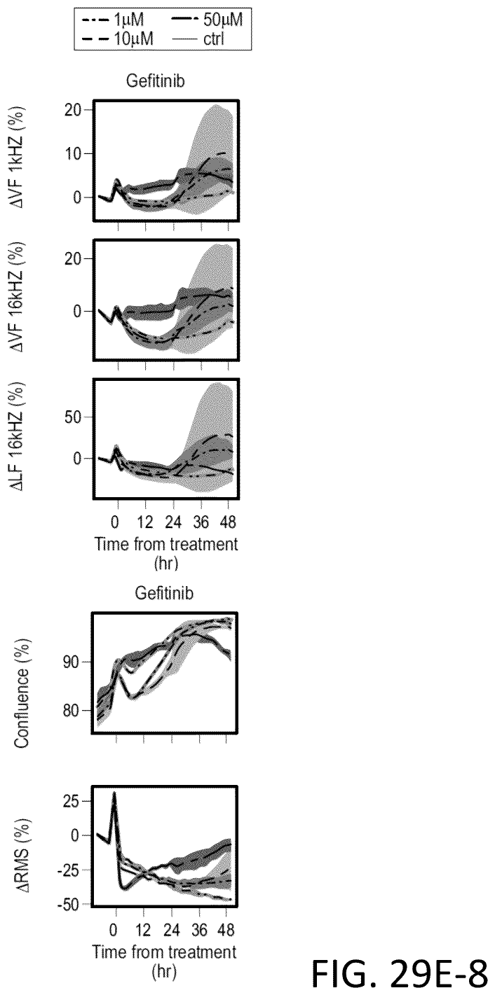

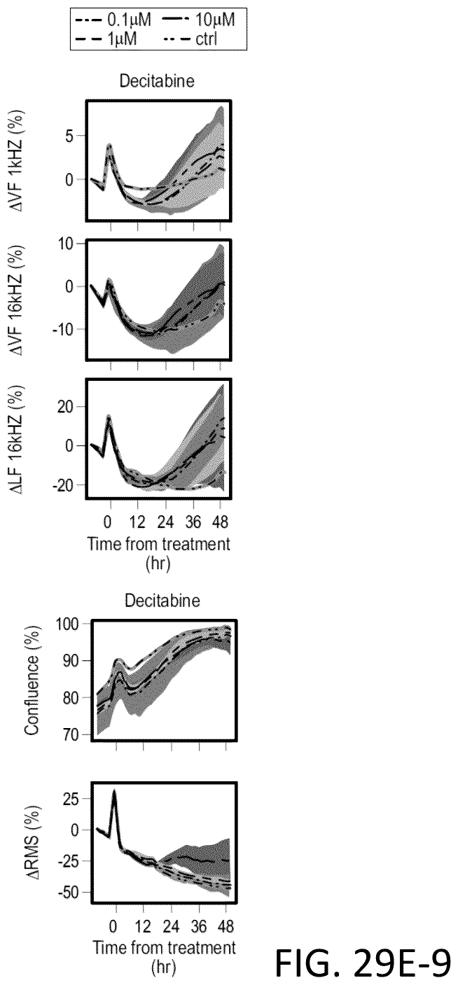

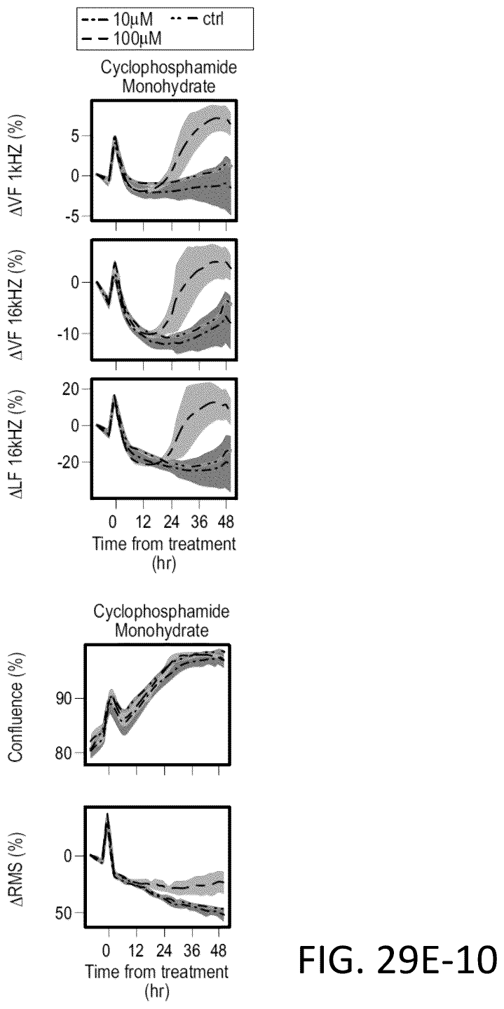

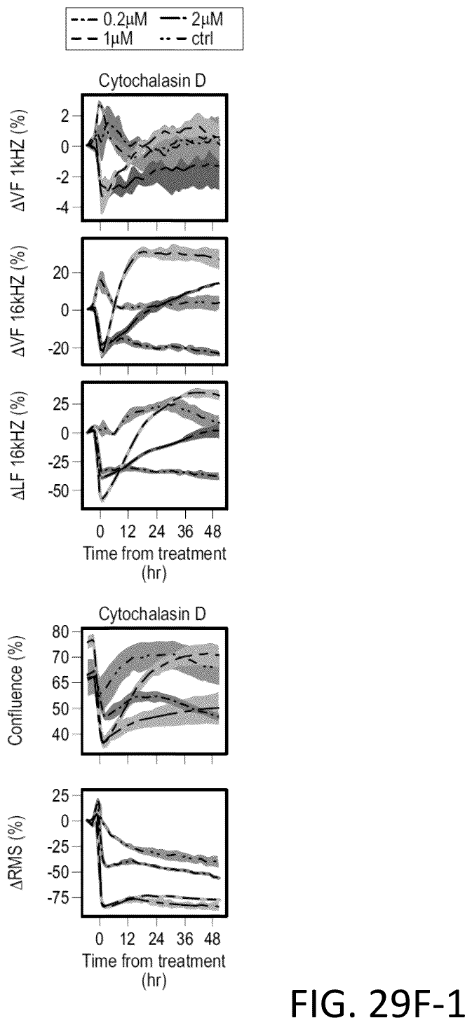

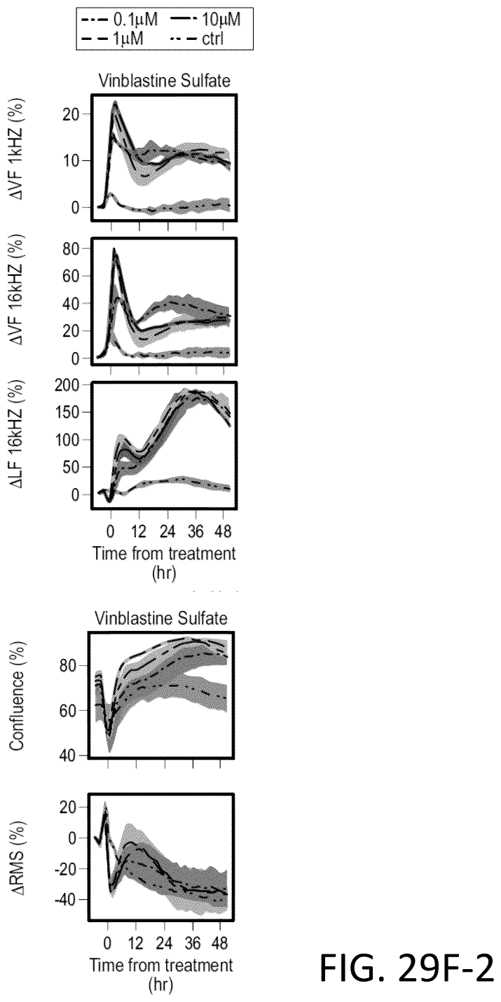

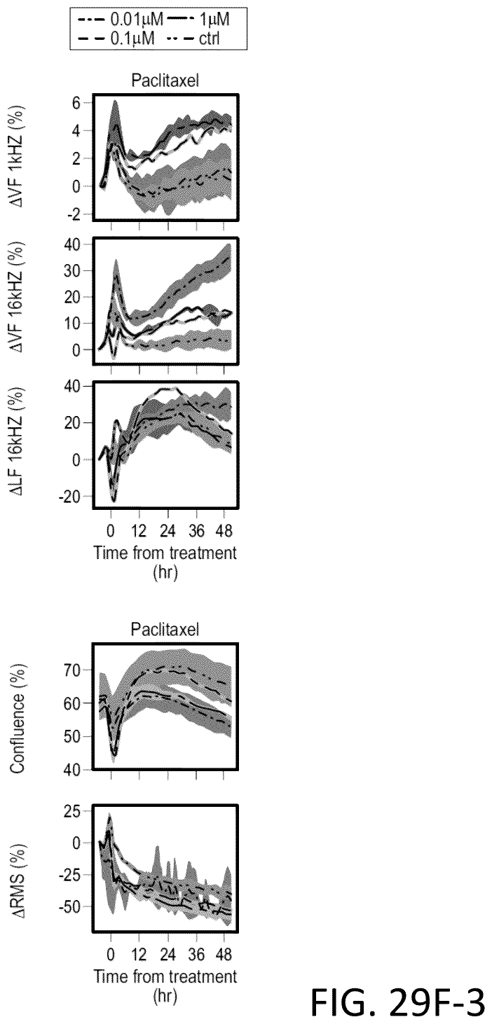

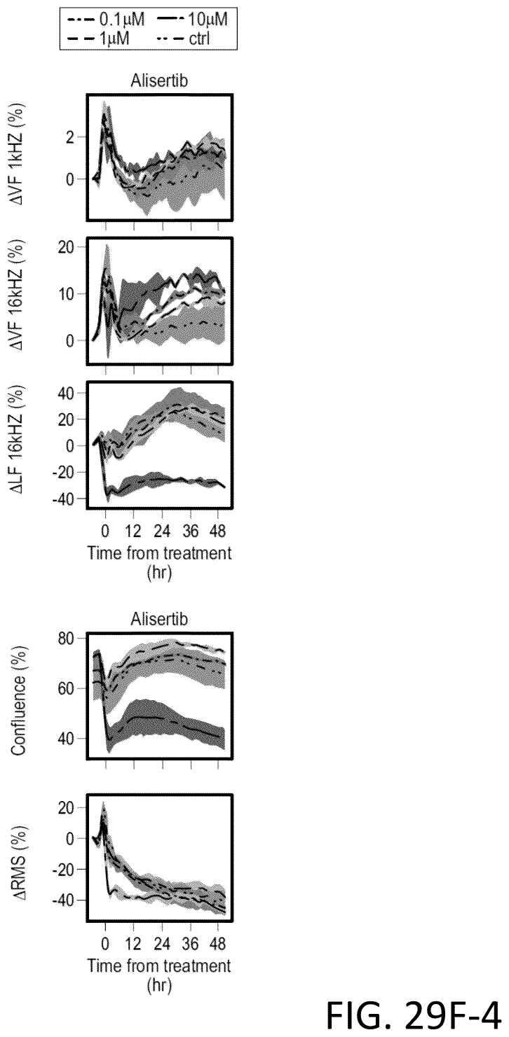

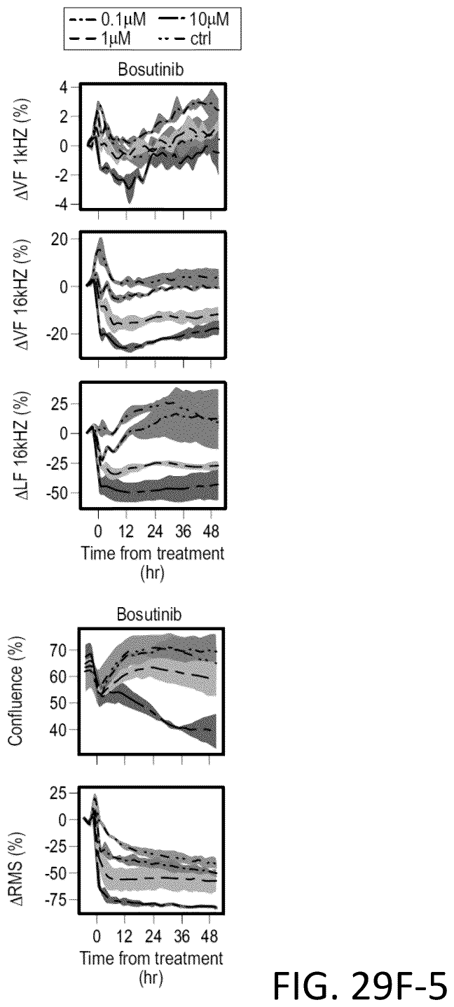

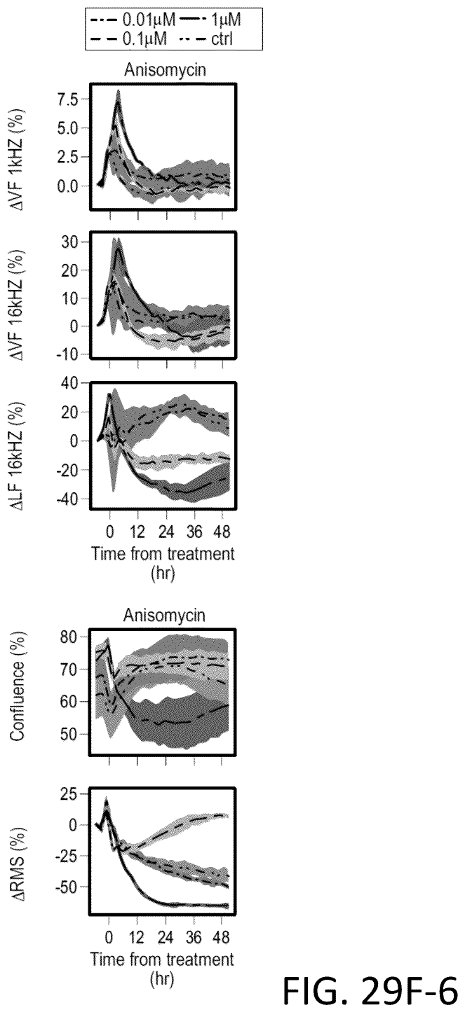

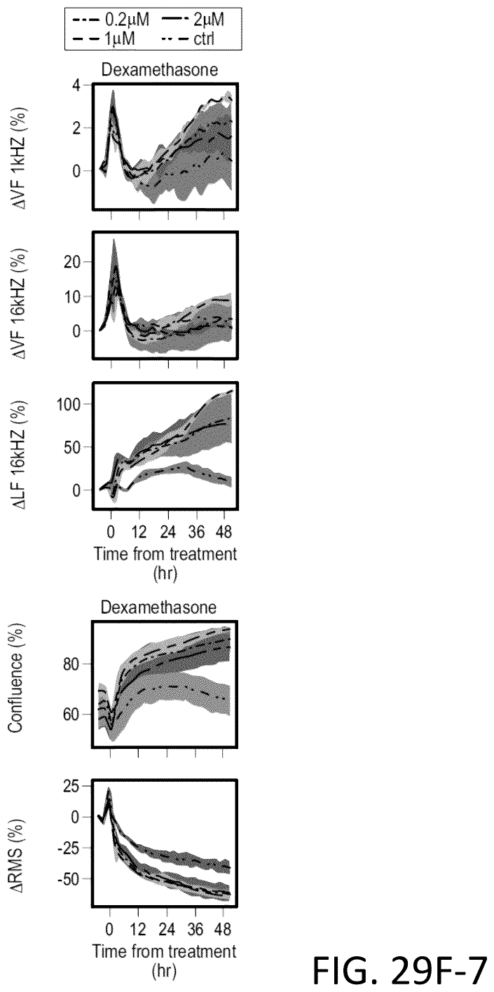

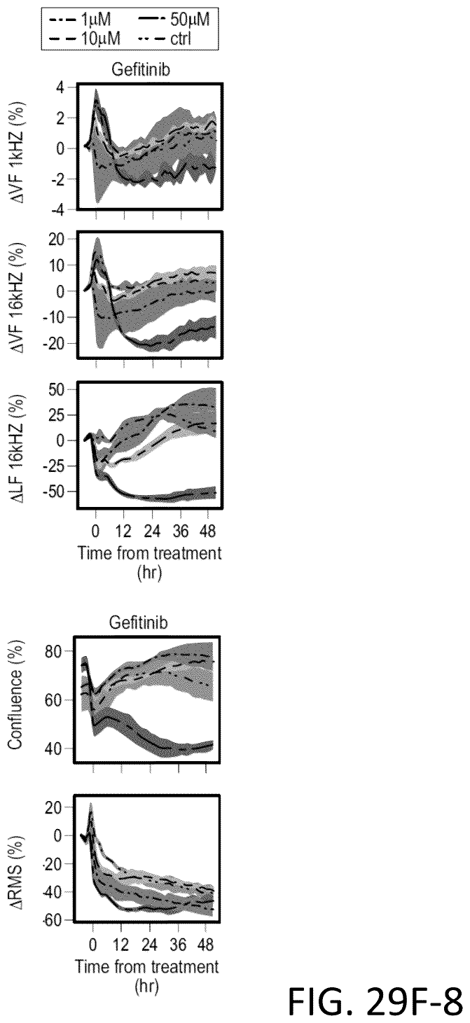

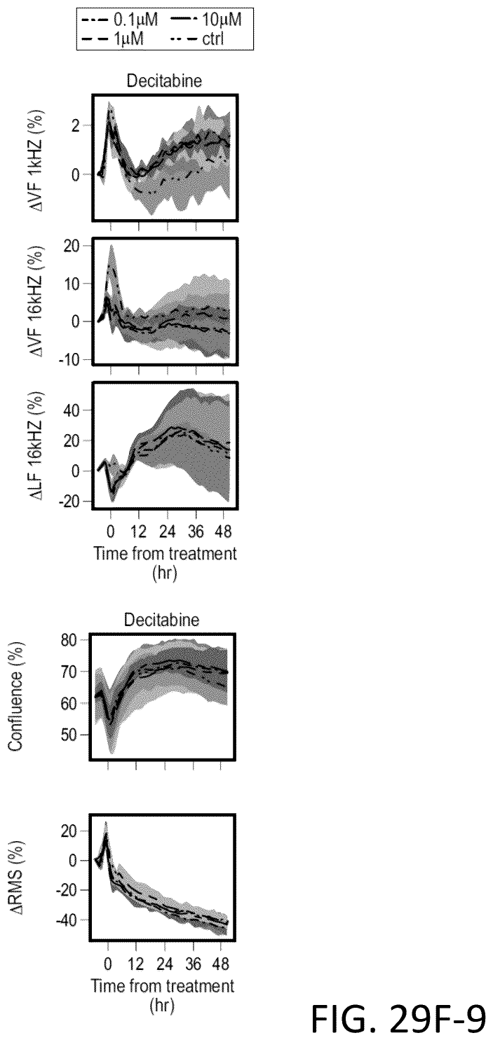

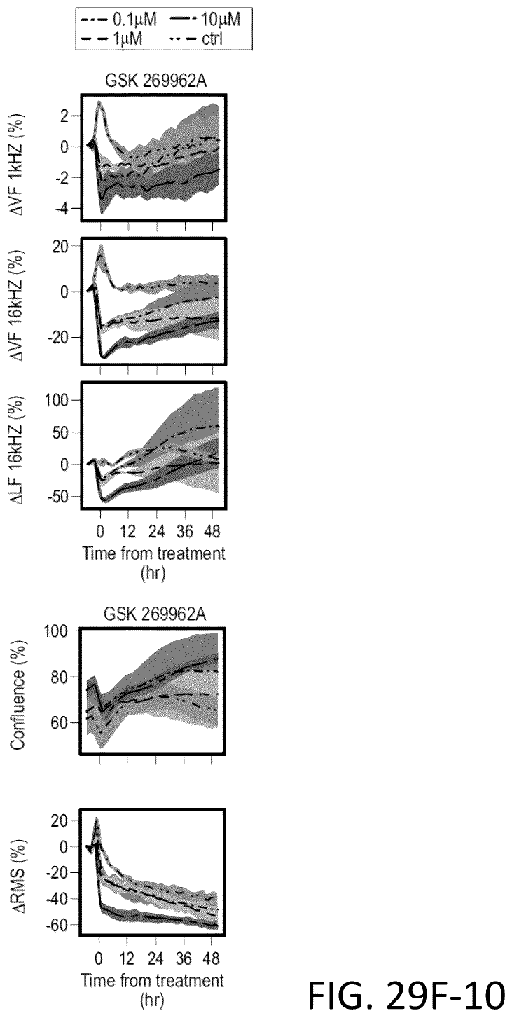

In the MDCK drug treatments, it was found that Alisertib, an Aurora kinase inhibitor, accelerated transepithelial water transport as evidenced by a sharp decrease in cell surface attachment and increased doming, as shown in FIGS. 29A-C. In contrast, Bosutinib, a multi-kinase inhibitor, decelerated water transport as evidenced by a slower decrease of cell attachment and no domes. Interestingly, Alisertib has been shown to exacerbate ADPKD in animal models, while Bosutinib was in Phase II clinical trials for treatment of ADPKD, matching the differential signatures of the MDCK experiment.

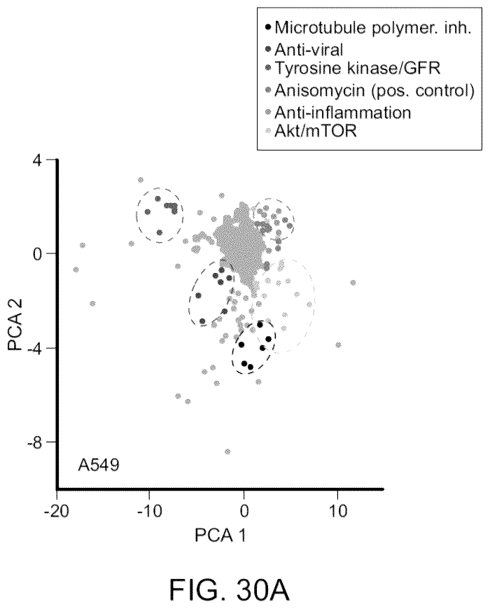

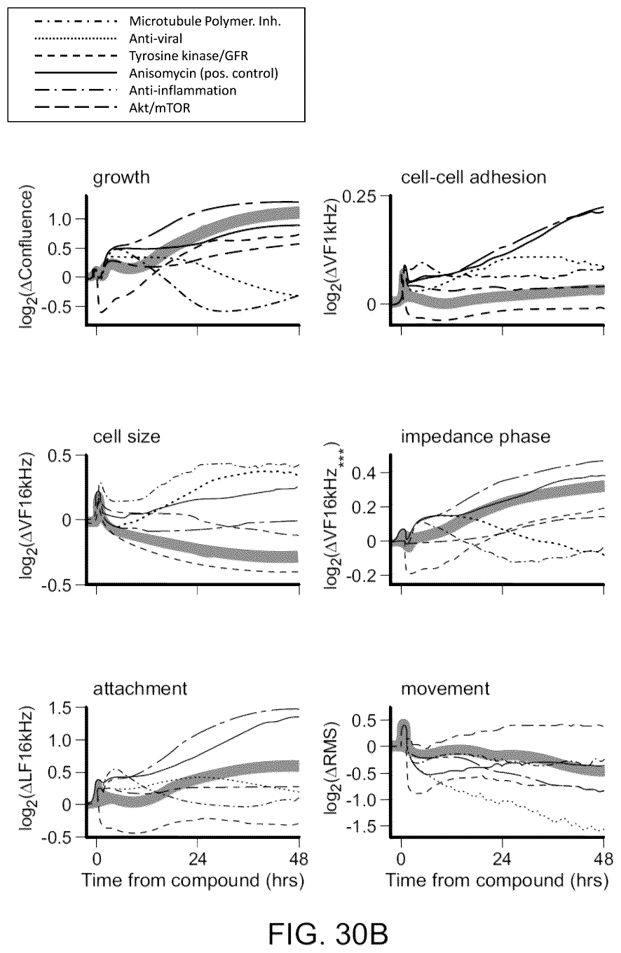

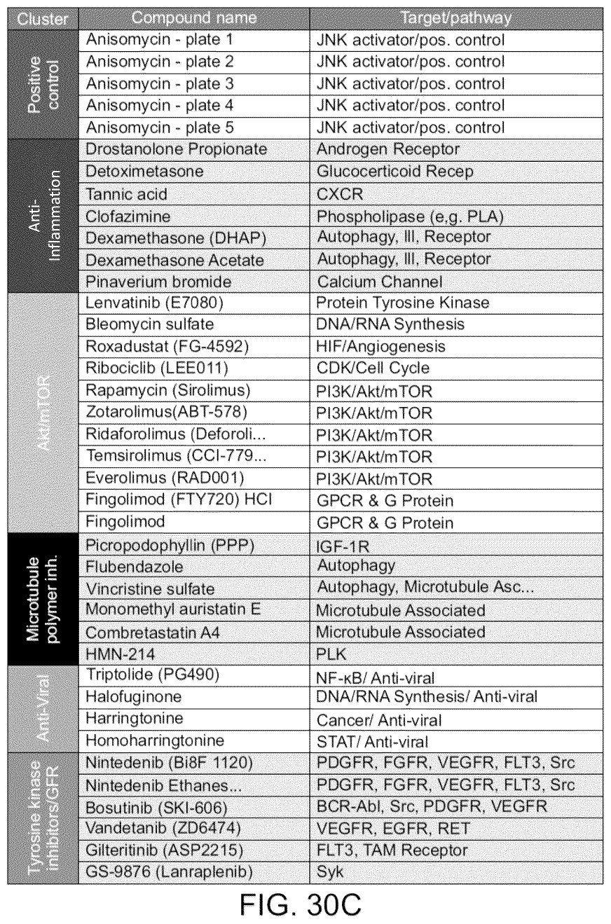

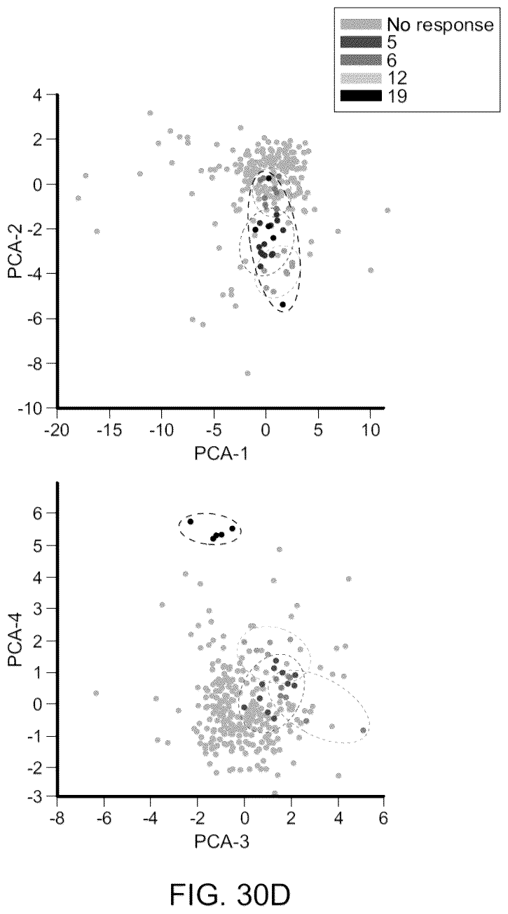

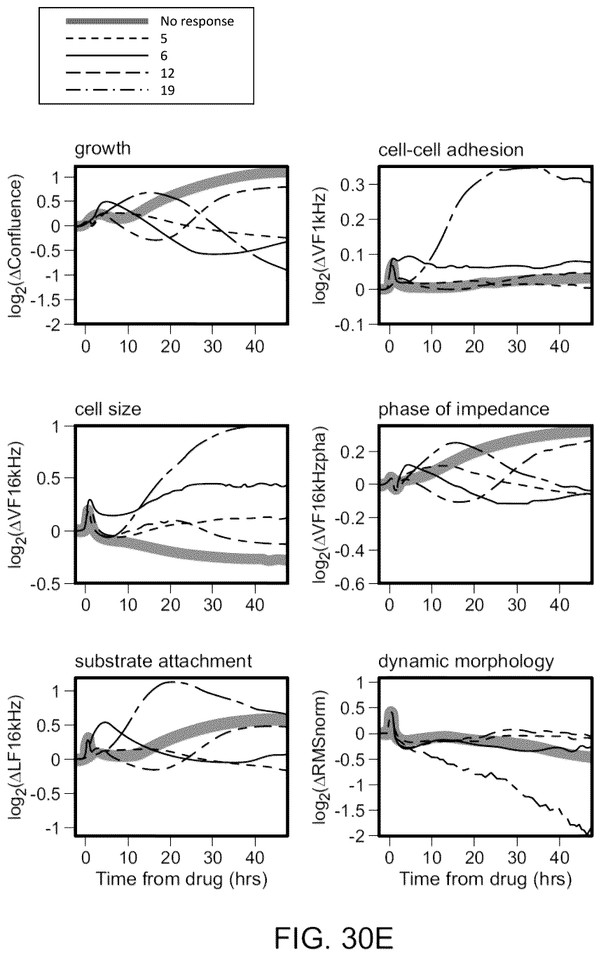

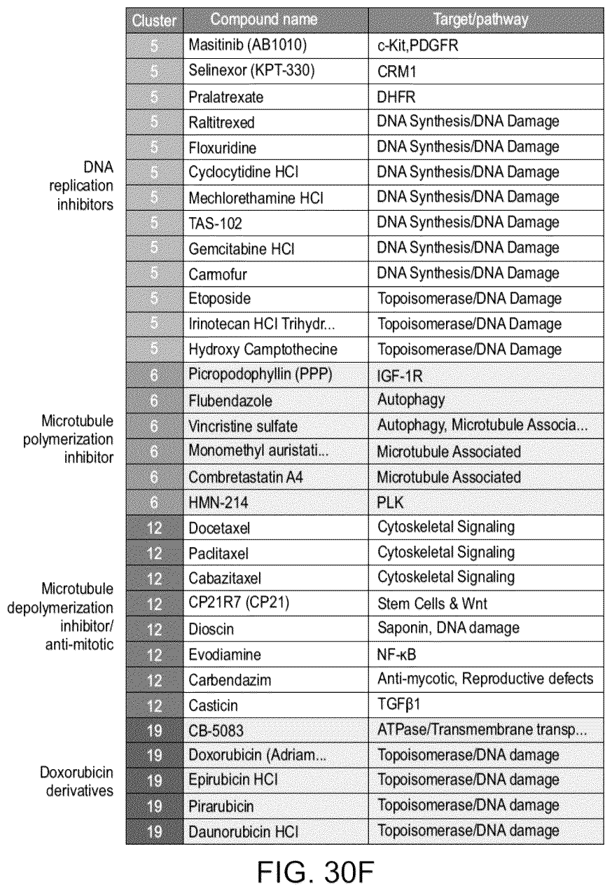

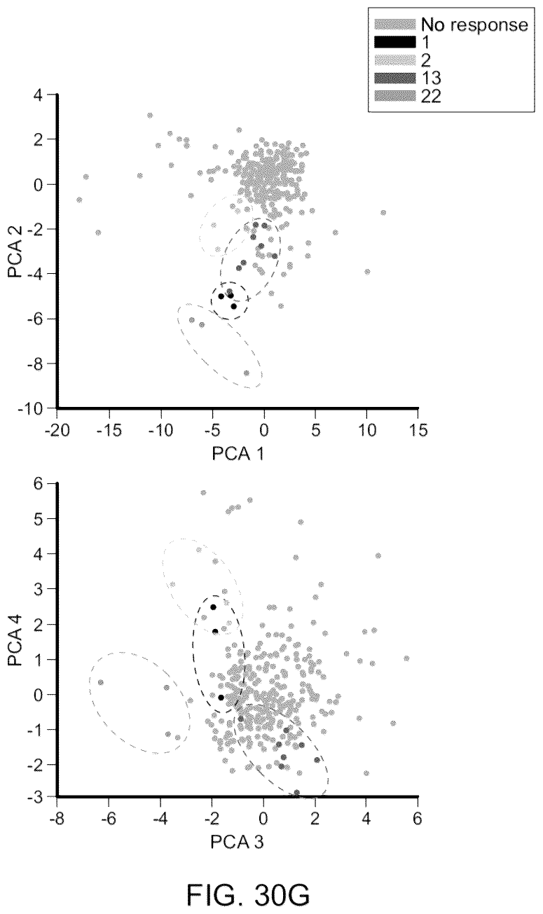

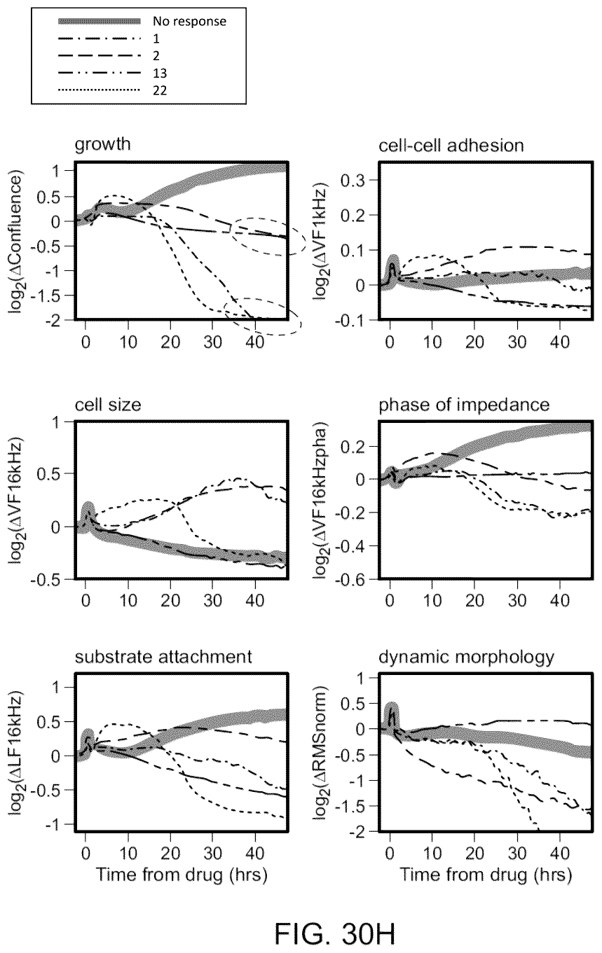

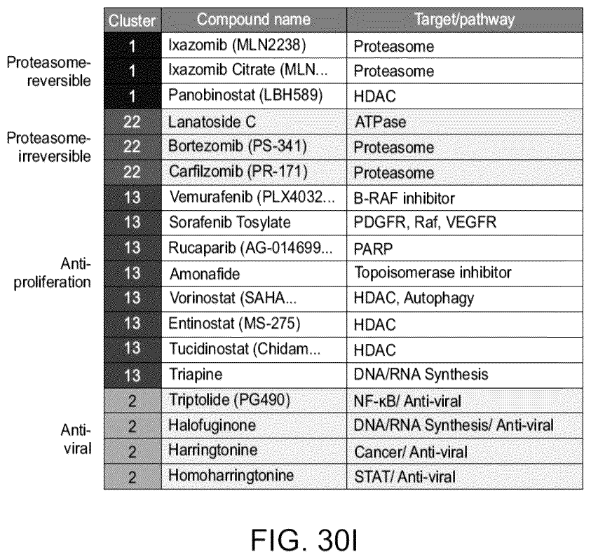

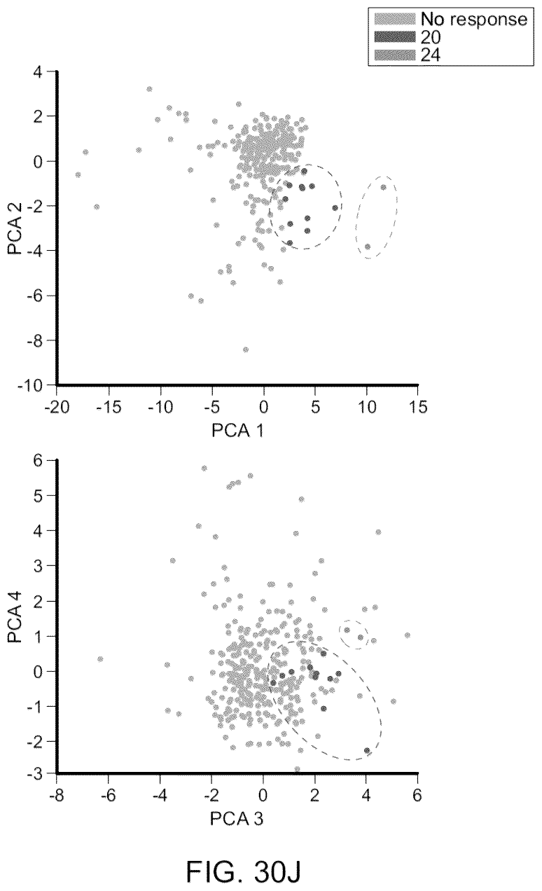

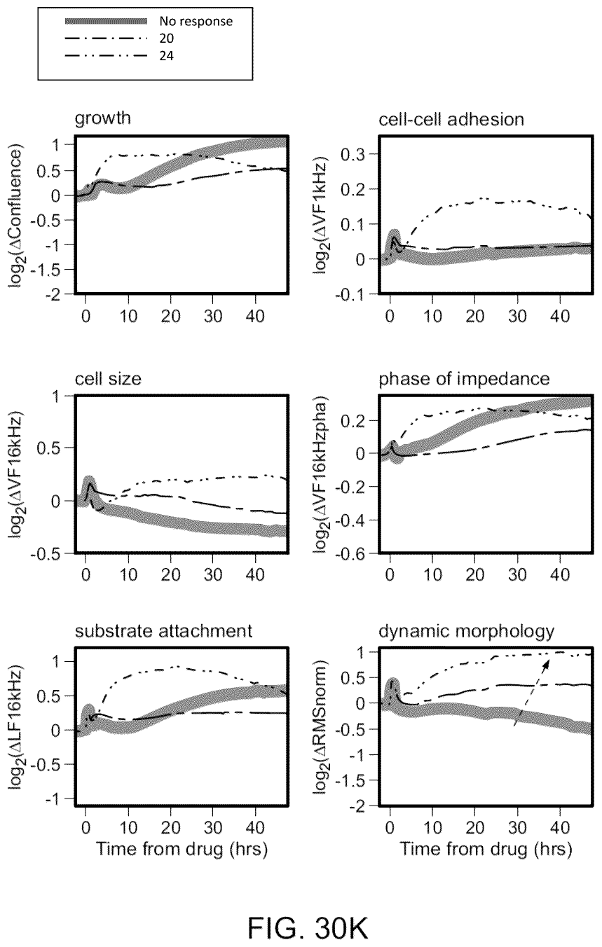

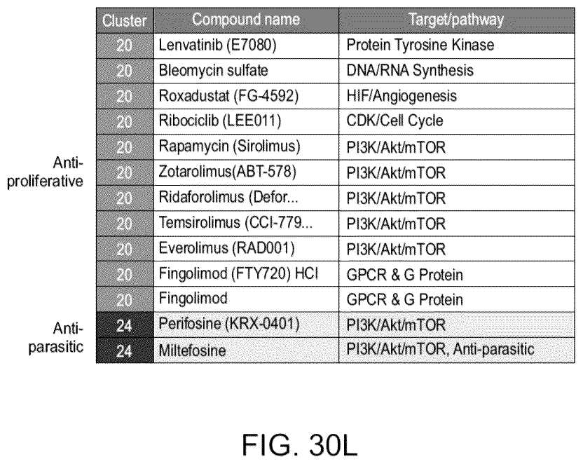

In the second approach, rather than comparing 5 measurement parameters, all field and calculation parameters were used to perform an unbiased phenotypic profiling with a larger compound library. A total of 341 compounds were chosen as a sub-set of an FDA approved library and applied on two cell types, A549 and Caco-2. At least two compounds per target were chosen for conserved phenotype effects. For the screens, compounds were added 24 or 48 hours post cell seeding and measured up to 48 hours after the compound addition. Time normalization, a principal component analysis (PCA), and unbiased clustering were sequentially performed on the high-dimensional data readouts (VF & LF magnitudes, phase, and DC). This unbiased approach ensured compound separation based solely on functional and morphological effects of the cells, with no bias of the targets/pathways of the compounds. Positive controls across the plates helped assess the variance in the data and validated the clustering; Anisomycin was chosen as a positive control for the A549 (separated into its own cluster in FIG. 30A) as it showed increases in VF across many cell types while Anisomycin, Bosutinib, and YM-201636 were used as positive controls for the Caco-2.

A549 has been shown to be an effective cell model for high-content phenotypic screening. The A549 clustering analysis revealed interesting insights about MOA of various compounds and their effects on live-cell function. A cluster was identified consisting of several anti-inflammatory compounds, as shown in FIG. 30C, which separated in the PCA due to an increase in cell-cell adhesion, as shown in FIG. 30B. This cluster contained two forms of Dexamethasone which has been described to reduce inflammation and increase the tissue barrier in A549 cells, corresponding with our measured MOA signature. Multiple clusters were related to cell death or growth inhibition. The end-point effects of these compounds on growth were often very similar, however, distinct transient effects on cell morphology separated compounds into different clusters. These were reflective of the MOA of the compounds in that cluster. For example, a microtubule polymerization inhibitor cluster and anti-viral cluster exhibited a similar reduction of confluency (−20% over the 48 hours), yet they separated due to distinct differences in the other parameters such as attachment and movement.

In an extended analysis, additional clusters identifying DNA replication inhibitors, microtubule depolymerization inhibitors, antimetabolites, anti-proliferation, and reversible/irreversible proteasome inhibitors separated as distinct clusters based on their transient morphological effects. The additional results from the extended analysis are shown in FIGS. 30D-I. Thus, it is evident that the measurement device was sensitive enough to separate very closely related compounds based on their effects on cell state.

Some compounds clustered by target. For example, most Akt/mTOR inhibitors had a similar effect on cell function. Interestingly, while these compounds had relatively minor effects on many morphological properties of cells, it is one of the few clusters that caused an increase in movement (RMS). FIGS. 30J-L show results from the cluster analysis on A549 cells, with two Akt/mTOR clusters observed.

In another example, a common annotated function was not evident, such as the anti-viral cluster containing compounds that target NF-KB, DNA synthesis and STAT. However, upon deeper investigation, all the compounds in the cluster were identified as functional hits during an anti-viral phenotypic screen for SARS-CoV-2 suggesting common underlying function.

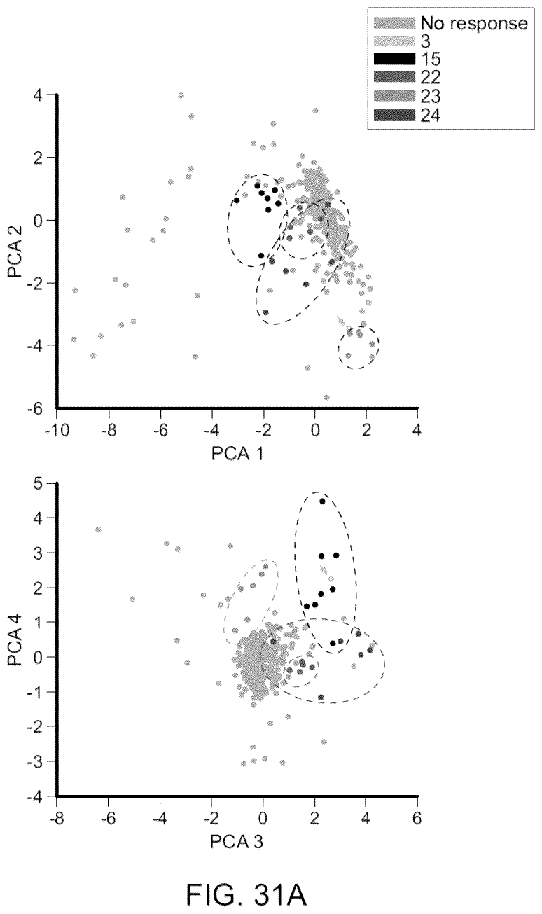

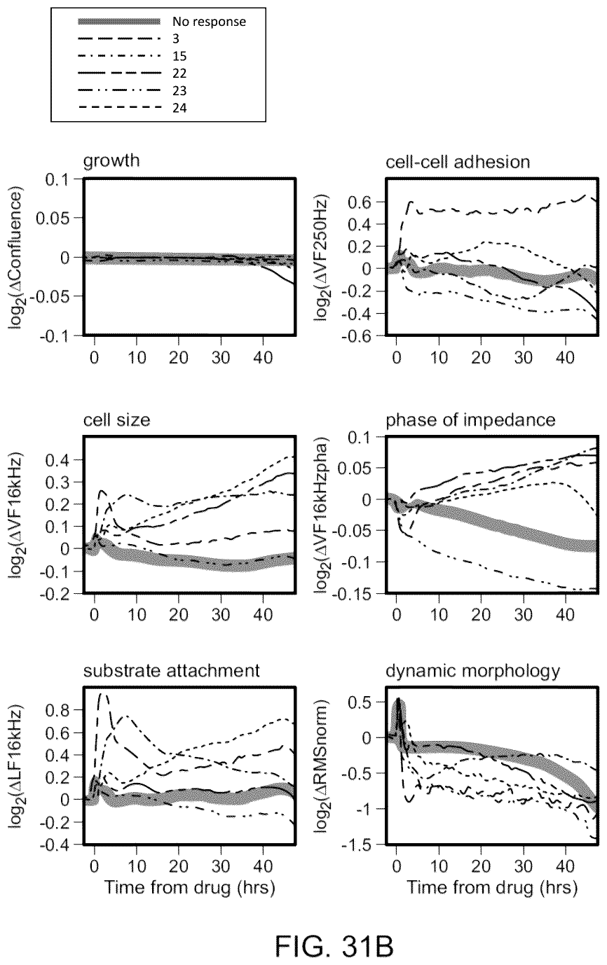

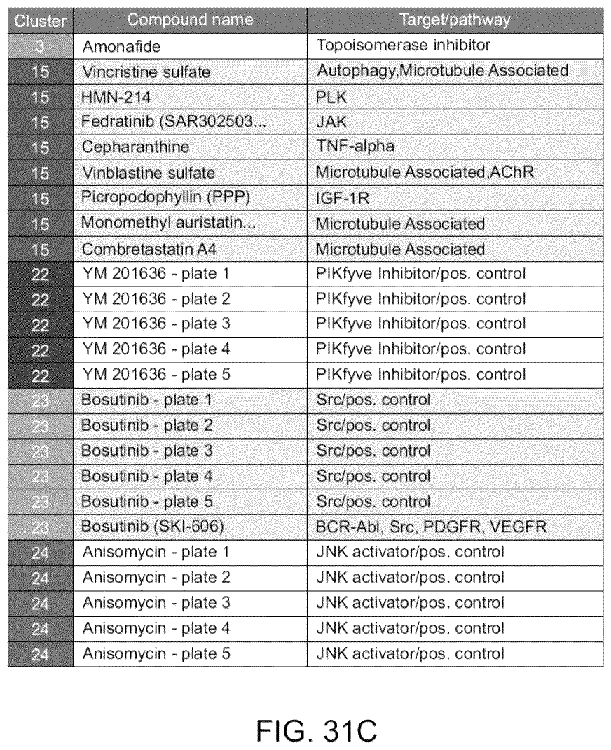

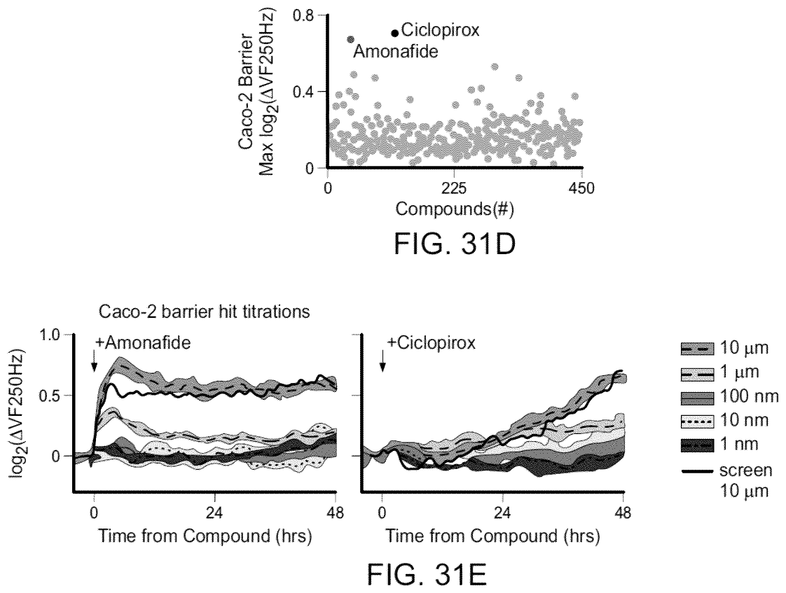

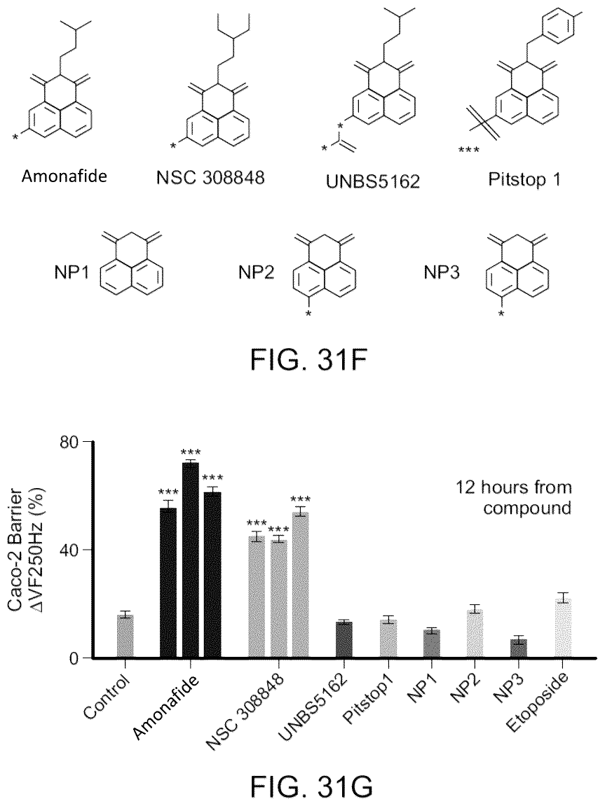

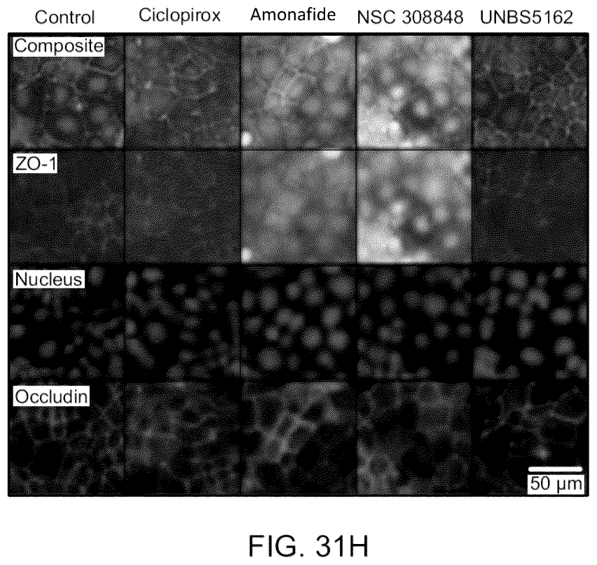

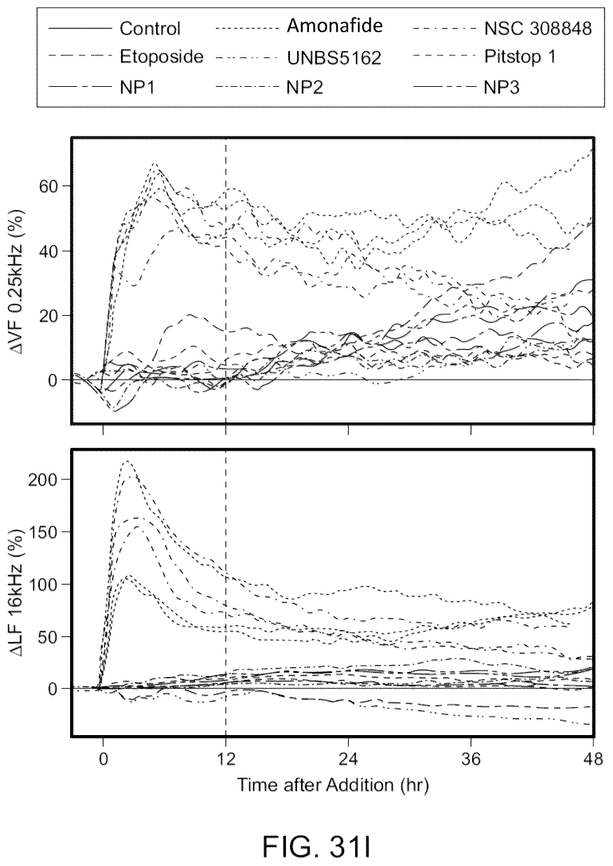

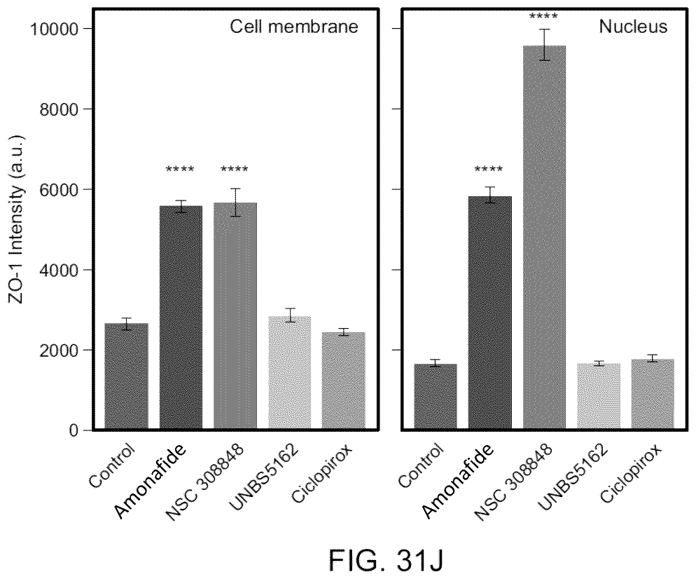

Caco-2 cells are widely used to model intestinal epithelia barrier function and study modulators of tight junction in epithelia. A clustering analysis similar to the one described above for A549 cells was performed for Caco-2 cells. The results are shown in FIGS. 31A-C.

The Caco-2 screen that was performed identified Amonafide, a DNA intercalating agent and Topoisomerase II inhibitor, and Ciclopirox, an antifungal iron chelator, as compounds that caused the largest barrier increase over the 48-hours, as shown in FIG. 31D. A subsequent dose response experiment, the results of which are shown in FIG. 31E, confirmed the screen hits. Amonafide caused a rapid increase in barrier suggesting modulation through cell signaling or protein changes, while Ciclopirox caused a more gradual increase in barrier, suggesting longer term responses such as change in gene expression.

To further investigate the specificity and test the observed rapid response, commercially available analogues of Amonafide (shown in FIG. 31F) were acquired their effects on Caco-2 barrier were measured. FIG. 31 includes two graphs showing measurement signals detected from samples in specific wells treated with Amonafide or one of its analogues. Results measured for all samples in the wells are shown in FIG. 31G.

Only one of the derivatives, NSC 308848, showed a similar increase in barrier. Interestingly, UNBS5162, a structurally similar compound that retains the DNA intercalation activity did not affect the barrier. Similarly, Etoposide, a Topoisomerase II inhibitor that does not intercalate DNA also did not show the rapid barrier increase. This points to the observed increase in barrier being non-random, and potentially independent of both its DNA intercalation and Topoisomerase inhibitor activity.

To explore whether the increase in barrier was also reflected in changes at tight junctions, immunofluorescence imaging was performed to look at tight junction proteins ZO-1 and Occludin. Representative images obtained are shown in FIG. 31H, and the results of quantification of the measured ZO-1 immunofluorescence signal are shown in FIG. 31J. Amonafide treatment led to an increase in membrane ZO-1 levels, with additional nuclear localization. NSC 308848 had a similar effect on ZO-1 expression, while UNBS5162, that did not show the barrier increase, did not affect ZO-1 levels or distribution. This suggests that the change in ZO-1 was directly related to the observed increase in barrier. Interestingly, although Ciclopirox increased the barrier, it did not affect ZO-1 distribution or expression and likely tightened barriers through a different mechanism. Therefore, the real-time data was able to identify two different MOAs of barrier tightening, that were further validated by immunofluorescence data.

ZO-1 has been shown to relocate to the nucleus during remodeling of tight junctions and its nuclear vs. membrane localization is related to cell-signaling. Taken together, the rapid and sustained increase in the measured barrier of the Caco-2 sheet with the increase in ZO-1 expression and localization towards the nucleus, strongly suggested a direct effect of Amonafide on tight junctions, potentially through activating signaling pathways. Furthermore, the similar response measured for NSC 208848 and not UNBS5162 or Etoposide, suggested an off-target MOA other than its primary pharmacological action as an intercalating agent and Topoisomerase II inhibitor.

In summary, the combination of multiple field and frequency impedance parameters allowed for measurement of a label-free, high-dimensional electrical representation of cell state, based in part on morphology and cell function. Real-time measurements allowed state changes to be observed over time. This generated a wealth of information on the intermediate cell states in a drug response. Determining cell death MOAs is a primary application for these capabilities: as cell death is innately a physical process, the measurement devices described herein are highly sensitive to the morphology of cell death and its “order of operations.” This label-free, MOA profiling capability is widely useful throughout in vitro toxicology, which heavily relies upon end-point readouts that are blind to cell state transitions and potential off-target activities.

To complement the breadth of information for MOA assessment, many of the functional parameters measured cannot currently be measured accurately nor at scale by other technologies. Thus, the measurement devices described herein can be used to enable novel therapeutic approaches in many disease areas. For example, the measurement devices described herein provide the only functional readout for water transport. The strong correlation of the compound responses on the water transport phenotype of MDCK cells (see, for example, FIGS. 29A-C) and ADPKD animal and human studies provide avenues for ADPKD screening applications.

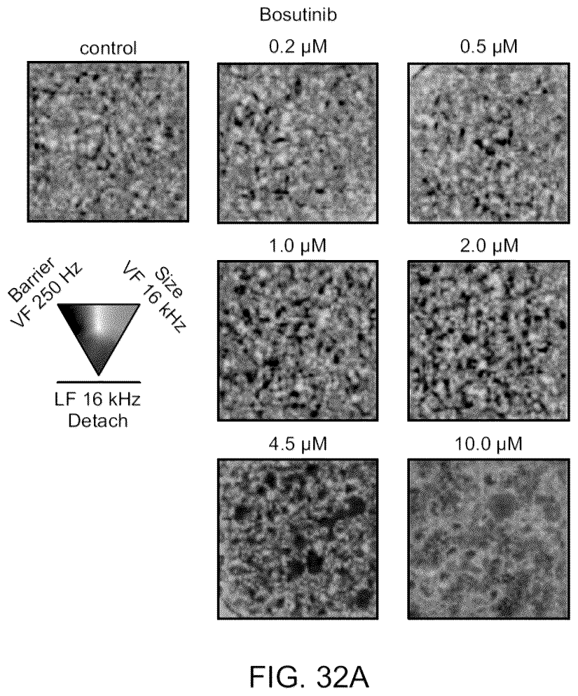

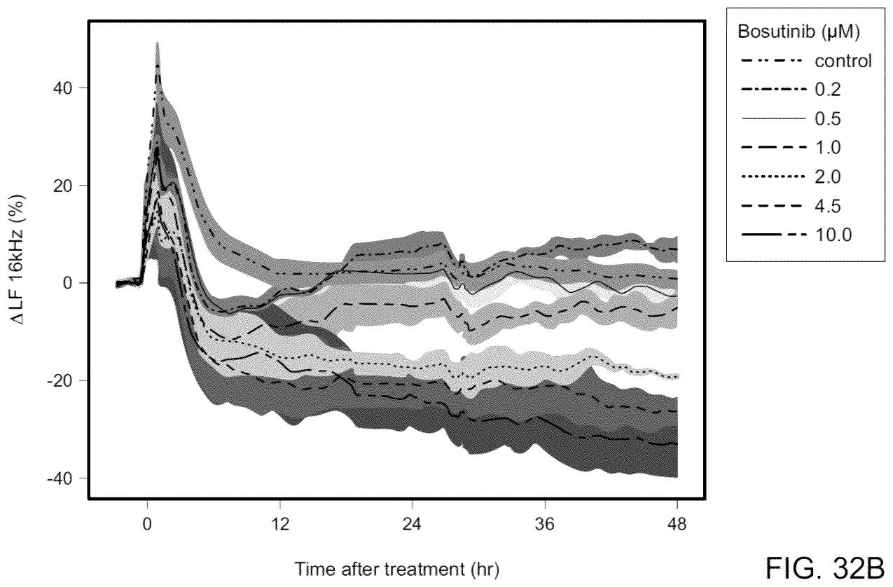

Beyond the kidney, doming as evidence of water transport is observed in other cell types, including Caco-2 and Calu-3. FIGS. 32A-B show results for Caco-2 cells treated with Bosutinib at various concentrations. Evidence of doming was observed starting at about 40 hours post-addition for the 10 μM Bosutinib dose. These additional cell types can be used to create effective phenotypic models for other diseases, such as chronic diarrhea or cystic fibrosis. The water transport phenotype is also of great interest in functional aquaporin inhibitor screening, a drug discovery area spanning oedema, cancer, obesity, brain injury, glaucoma, and other conditions, which lacks a reliable assay.

Epithelial leakiness, or barrier dysfunction, occurs from physical damage or modifications to tight junctions and is important in the context of inflammatory bowel disease (IBD), celiac disease, Crohn's disease, and other diseases of the gut. Most therapeutic approaches to IBD have focused on anti-inflammatory or immune-suppressive therapies, but more recently, direct tightening of the epithelial barrier is being explored as a therapeutic approach. The screening methods described herein using Caco-2 cells revealed a potentially new MOA for increasing barrier and demonstrated the power of screening against physiologically relevant parameters in higher throughputs. Amonafide has inherent toxicity and is not a good therapeutic candidate for IBD as a result. However, the study of analogues discussed above demonstrated that the measurement devices and methods described herein has the sensitivity for an in-depth medicinal chemistry, structure activity relationship (SAR) approach for developing lead compounds. The expansion of compounds screened can also be used to identify additional modulators of intestinal barrier as potential therapeutic molecules.

1. A measurement device, comprising:
a substrate;
a plurality of integrated circuits, each comprising a plurality of electrodes, and the plurality of integrated circuits being positioned on the substrate in a two-dimensional array comprising n rows and m columns;
a communication interface connected to each of the plurality of integrated circuits so that each integrated circuit is addressable through the interface; and
a data output interface comprising multiple data output lines connected to the communication interface,
wherein in each of the n rows of the array, the integrated circuits are connected to a common data line; and
wherein the measurement device is configurable, responsive to a control signal, to transmit measurement information through the data output interface by, in one or more of the n rows of the array:
selectively transmitting measurement information from one of the integrated circuits connected to the row's common data line to the data output interface; and
inactivating one or more of the other integrated circuits connected to the row's common data line.

2. The measurement device of claim 1, wherein the measurement device is configurable, responsive to a control signal, to transmit measurement information through the data output interface by, in each of the n rows of the array:
selectively transmitting measurement information from one of the integrated circuits connected to the row's common data line to the data output interface; and
inactivating one or more of the other integrated circuits connected to the row's common data line.

3. The measurement device of claim 1, wherein the measurement device is configurable, responsive to a control signal, to selectively configure one of the integrated circuits in one or more of the n rows of the array to prepare the selectively configured integrated circuit for transmission of measurement information during transmission of measurement information from the one of the integrated circuits in the one or more of the n rows of the array.

4. The measurement device of claim 3, wherein the measurement device is configurable, responsive to a control signal, to selectively configure one of the integrated circuits in each of the n rows of the array to prepare the selectively configured integrated circuit for transmission of measurement information during transmission of measurement information from the one of the integrated circuits in each of the n rows of the array.

5. The measurement device of claim 1, wherein n is greater than or equal to 4 and less than or equal to 24, and wherein m is greater than or equal to 4 and less than or equal to 24.

6-7. (canceled)

8. The measurement device of claim 1, wherein the multiple data output lines comprise multiple low voltage differential signaling (LVDS) data output lines.

9. The measurement device of claim 1, wherein each integrated circuit comprises at least one analog to digital converter (ADC) and at least one peripheral circuit (PC).

10. The measurement device of claim 9, wherein the peripheral circuit is configurable, responsive to a control signal, to operate in a current measurement mode in which the peripheral circuit comprises a trans-impedance amplifier (TIA) that amplifies a current signal from an electrode.

11. The measurement device of claim 9, wherein the peripheral circuit is configurable, responsive to a control signal, to operate in a voltage measurement mode in which the peripheral circuit comprises a capacitive gain voltage amplifier.

12. The measurement device of claim 9, wherein the peripheral circuit is configurable, responsive to a control signal, to operate in a direct voltage drive mode in which the peripheral circuit applies a voltage signal to an electrode of an integrated circuit.

13. The measurement device of claim 9, wherein the peripheral circuit is configurable, responsive to a control signal, to operate in a buffered voltage drive mode in which the peripheral circuit receives a voltage signal, passes the voltage signal through an amplifier to generate a buffered voltage signal, and applies the buffered voltage signal to an electrode of the integrated circuit.

14. The measurement device of claim 1, wherein the measurement device is configurable, responsive to a clock signal generated external to the measurement device, to operate each of the integrated circuits synchronously with respect to the clock signal.

15. The measurement device of claim 1, wherein inactivating one or more of the integrated circuits comprises configuring the one or more integrated circuits to operate in a power state in which an amount of power supplied to the one or more integrated circuits is reduced relative to an amount of power applied to integrated circuits that are selectively transmitting measurement information.

16. The measurement device of claim 1, wherein inactivating one or more of the integrated circuits comprises providing, on one or more output lines of the one or more of the integrated circuits, an impedance that is higher than an impedance on one or more output lines of integrated circuits that are selectively transmitting measurement information.

17. The measurement device of claim 1, wherein each integrated circuit in the array comprises:
a unique array address; and
a configuration interface comprising at least one configuration line,
wherein the configuration interface is a serial peripheral interface (SPI).

18. The measurement device of claim 17, wherein the measurement device is configurable, responsive to a received first control signal comprising one or more addresses corresponding to the one or more of the other integrated circuits, to transmit an inactivation control signal selectively to the one or more of the other integrated circuits to inactivate the one or more of the other integrated circuits.

19. The measurement device of claim 18, wherein the measurement device is configurable to transmit the inactivation control signal responsive to a received second control signal that corresponds to the inactivation control signal.

20. A system, comprising:
the measurement device of claim 1; and
a host controller comprising a host interface configured to connect to the communication interface,
wherein the host controller comprises instructions that, when executed by the host controller, cause the host controller to obtain measurement data from the measurement device by transmitting the control signal to the measurement device.

21. The measurement device of claim 1, comprising a partitioning member attached to a surface of the measurement device and comprising a plurality of apertures, wherein the plurality of apertures are dimensioned and positioned to individually surround the integrated circuits of the measurement device.

22. The measurement device of claim 21, wherein the plurality of apertures fully enclose the sensors of the measurement device to form a plurality of wells on the surface of the measurement device.

23-25. (canceled)

26. A system, comprising:
the measurement device of claim 1; and
a housing comprising a plurality of receiving bays, wherein each one of the receiving bays is configured to receive the measurement device.

27. The system of claim 26, comprising a host controller integrated within the housing, wherein each one of the receiving bays of the housing comprises a connector dimensioned to engage with a connector of the measurement device so that when the measurement device is received within a receiving bay of the housing, the communication interface of the measurement device is connected to a host interface of the host controller.

28. (canceled)

29. A method, comprising:
providing a measurement device comprising:
a substrate;
a plurality of integrated circuits, each comprising a plurality of electrodes, and the plurality of integrated circuits being positioned on the substrate in a two-dimensional array comprising n rows and m columns;
a communication interface connected to each of the plurality of integrated circuits so that each integrated circuit is addressable through the interface; and
a data output interface comprising multiple data output lines connected to the communication interface,
wherein in each of the n rows of the array, the integrated circuits are connected to a common data line;
selectively transmitting measurement information from one of the integrated circuits connected to the row's common data line to the data output interface; and
inactivating one or more of the other integrated circuits connected to the row's common data line.

30. The method of claim 29, comprising transmitting measurement information through the data output interface by, in each of the n rows of the array:
selectively transmitting measurement information from one of the integrated circuits connected to the row's common data line to the data output interface; and
inactivating one or more of the other integrated circuits connected to the row's common data line.

31. The method of claim 29, comprising selectively configuring one of the integrated circuits in one or more of the n rows of the array to prepare the selectively configured integrated circuit for transmission of measurement information during transmission of measurement information from the one of the integrated circuits in the one or more of the n rows of the array.

32. The method of claim 31, comprising selectively configuring one of the integrated circuits in each of the n rows of the array to prepare the selectively configured integrated circuit for transmission of measurement information during transmission of measurement information from the one of the integrated circuits in each of the n rows of the array.

33. The method of claim 29, wherein n is greater than or equal to 4 and less than or equal to 24, and wherein m is greater than or equal to 4 and less than or equal to 24.

34-35. (canceled)

36. The method of claim 29, wherein the multiple data output lines comprise multiple low voltage differential signaling (LVDS) data output lines.

37. The method of claim 29, wherein each integrated circuit comprises at least one analog to digital converter (ADC) and at least one peripheral circuit (PC).

38. The method of claim 37, comprising operating at least one peripheral circuit of at least one integrated circuit in a current measurement mode in which the at least one peripheral circuit comprises a trans-impedance amplifier (TIA) that amplifies a current signal from an electrode.

39. The method of claim 37, comprising operating at least one peripheral circuit of at least one integrated circuit in a voltage measurement mode in which the at least one peripheral circuit comprises a capacitive gain voltage amplifier.

40. The method of claim 37, comprising operating at least one peripheral circuit of at least one integrated circuit in a direct voltage drive mode in which the at least one peripheral circuit applies a voltage signal to an electrode of an integrated circuit.

41. The method of claim 37, comprising operating at least one peripheral circuit of at least one integrated circuit in a buffered voltage drive mode in which the at least one peripheral circuit receives a voltage signal, passes the voltage signal through an amplifier to generate a buffered voltage signal, and applies the buffered voltage signal to an electrode of the at least one integrated circuit.

42. The method of claim 29, comprising receiving a clock signal generated external to the measurement device and operating each of the integrated circuits synchronously with respect to the clock signal.

43. The method of claim 29, comprising inactivating the one or more of the other integrated circuits by configuring the one or more other integrated circuits to operate in a power state in which an amount of power supplied to the one or more of the other integrated circuits is reduced relative to an amount of power applied to integrated circuits that are selectively transmitting measurement information.

44. The method of claim 29, comprising inactivating the one or more of the other integrated circuits by providing, on one or more output lines of the one or more of the other integrated circuits, an impedance that is higher than an impedance on one or more output lines of integrated circuits that are selectively transmitting measurement information.

45. The method of claim 29, wherein each integrated circuit in the array comprises a unique array address and a serial peripheral interface (SPI) configuration interface comprising at least one configuration line, the method further comprising:
receiving a first control signal comprising one or more addresses corresponding to the one or more of the other integrated circuits; and
transmitting an inactivation control signal selectively to the one or more of the other integrated circuits to inactivate the one or more of the other integrated circuits.

46. (canceled)

47. The method of claim 45, comprising receiving a second control signal, and transmitting the inactivation control signal in response to the received second control signal, wherein the received second control signal corresponds to the inactivation control signal.

48-332. (canceled)
